(19) United States
(12) Patent Application Publication
(10) Pub. No.: US 2009/0270443 A1
(43) Pub. Date: Oct. 29, 2009

(54) 1-AMINO IMIDAZO-CONTAINING COMPOUNDS AND METHODS

(76) Inventors: Doris Stoermer, White Bear Lake, MN (US); Luke T. Dressel, Somerset, WI (US); Karl J. Manske, Minneapolis, MN (US); George W. Griesgraber, Eagan, MN (US); Bernhard M. Zimmermann, Eagan, MN (US); Joseph F. Dellaria, JR., Woodbury, MN (US)

Correspondence Address: WOLF GREENFIELD & SACKS, P.C., 600 ATLANTIC AVENUE, BOSTON, MA, 02210-2206, US

(21) Appl. No.: 11/574,464

(22) PCT Filed: Sep. 2, 2005
(86) PCT No.: PCT/US05/31414

§ 371 Date: Feb. 28, 2007

Related U.S. Application Data
(60) Provisional application No. 60/606,548, filed on Sep. 2, 2004.

(51) Int. Cl. A61K 31/4375; C07D 471/04; A61K 31/437; C07D 471/14

(52) U.S. Cl. 514/293; 546/118; 514/303; 546/82

(57) ABSTRACT

Imidazo-containing compounds (e.g., imidazonaphthyridines, imidazopyridines) with an amino substituent at the 1-position, pharmaceutical compositions containing the compounds and methods of use of these compounds as immunomodulators, for inducing cytokine biosynthesis in animals and in the treatment of diseases including viral and neoplastic diseases are disclosed.

### RELATED APPLICATION

The present invention claims priority to U.S. Provisional Application Ser. No. 60/606,548, filed Sep. 2, 2004, which is incorporated herein by reference.

### BACKGROUND

In the 1950's the 1H-imidazo[4,5-c]quinoline ring system was developed, and 1-(6-methoxy-8-quinolinyl)-2-methyl-1H-imidazo[4,5-c]quinoline was synthesized for possible use as an antimalarial agent. Subsequently, syntheses of various substituted 1H-imidazo[4,5-c]quinolines were reported. For example, 1-[2-(4-piperidyl)ethyl]-1H-imidazo[4,5-c]quinoline was synthesized as a possible anticonvulsant and cardiovascular agent. Also, several 2-oxoimidazo[4,5-c]quinolines have been reported.

Certain 1H-imidazo[4,5-c]quinolin-4-amines and 1- and 2-substituted derivatives thereof were later found to be useful as antiviral agents, bronchodilators and immunomodulators. Subsequently, certain substituted 1H-imidazo[4,5-c]pyridin-4-amine, quinolin-4-amine, tetrahydroquinolin-4-amine, naphthyridin-4-amine, and tetrahydronaphthyridin-4-amine compounds as well as certain analogous thiazolo and oxazolo compounds were synthesized and found to be useful as immune response modifiers (IRMs), rendering them useful in the treatment of a variety of disorders.

There continues to be interest in and a need for compounds that have the ability to modulate the immune response, by induction of cytokine biosynthesis or other mechanisms.

### SUMMARY OF THE INVENTION

It has now been found that certain 1-amino 1H-imidazo-containing compounds modulate cytokine biosynthesis. In one aspect, the present invention provides compounds of the Formula I:

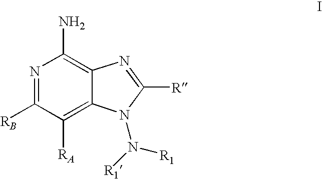

and more specifically the following compounds of the Formulas II, III, IV, V, VI, VII, VIII, IX, X, XI, and XII:

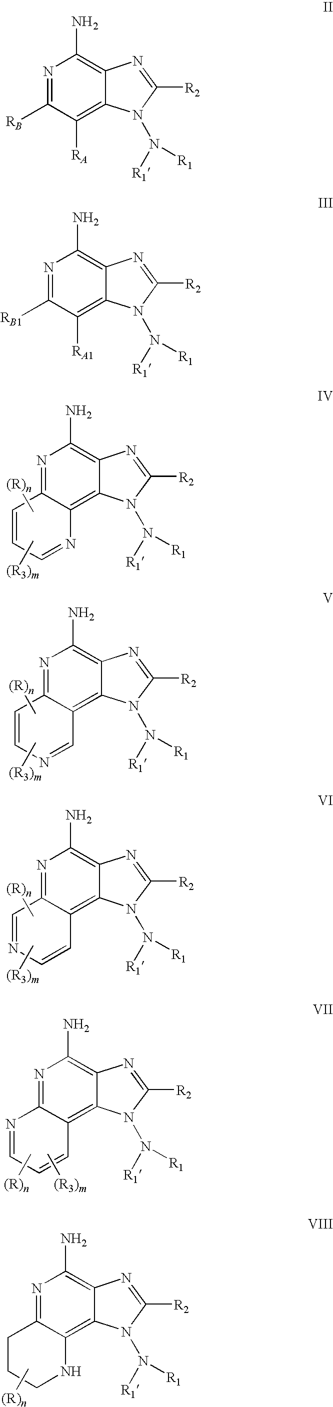

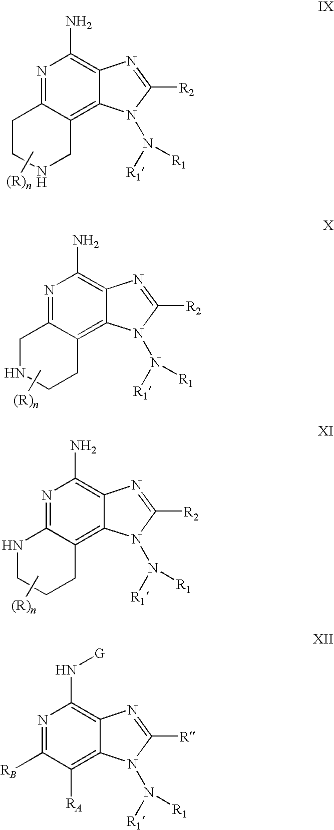

wherein R₁′, R₁, R₂, R₃, R″, R, R_{A}, R_{B}, R_{A1}, R_{B1}, G, m, and n are as defined below; and pharmaceutically acceptable salts thereof.

The compounds of Formulas III, II, IV, V, VI, VII, VIII, IX, X, XI, and XII are useful as immune response modifiers (IRMs) due to their ability to modulate cytokine biosynthesis (e.g., induce the biosynthesis or production of one or more cytokines) and otherwise modulate the immune response when administered to animals. Compounds can be tested per the test procedures described in the Examples Section. Compounds can be tested for induction of cytokine biosynthesis by incubating human peripheral blood mononuclear cells (PBMC) in a culture with the compound(s) at a concentration range of 30 to 0.014 μM and analyzing for interferon (α) or tumor necrosis factor (α) in the culture supernatant. The ability to modulate cytokine biosynthesis, for example, induce the biosynthesis of one or more cytokines, makes the compounds useful in the treatment of a variety of conditions such as viral diseases and neoplastic diseases, that are responsive to such changes in the immune response.

In another aspect, the present invention provides pharmaceutical compositions containing the immune response modifier compounds, and methods of inducing cytokine biosynthesis in animal cells, treating a viral disease in an animal, and/or treating a neoplastic disease in an animal by administering to the animal one or more compounds of the Formulas I, II, III, IV, V, VI, VII, VIII, IX, X, XI, and/or XII, and/or pharmaceutically acceptable salts thereof.

In another aspect, the invention provides methods of synthesizing the compounds of Formulas I, II, III, IV, V, VI, VII, VIII, IX, X, XI, and XII and intermediates useful in the synthesis of these compounds. One such intermediate is of the Formula XVII:

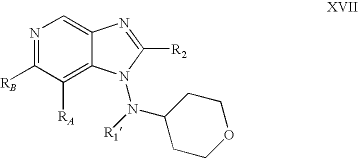

wherein R₁′, R₂, R_{A}, and R_{B} are as defined below.

As used herein, “a,” “an,” “the,” “at least one,” and “one or more” are used interchangeably.

The terms “comprising” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides compounds of the following Formulas I through XII and XVII:

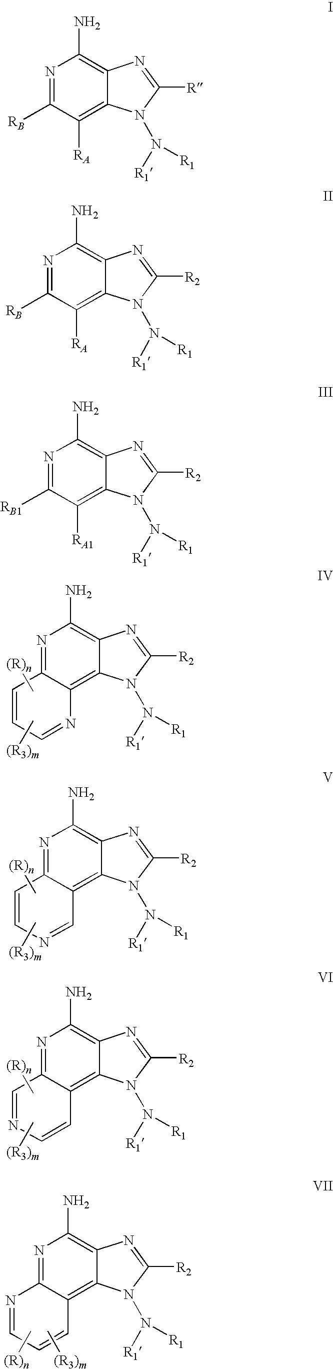

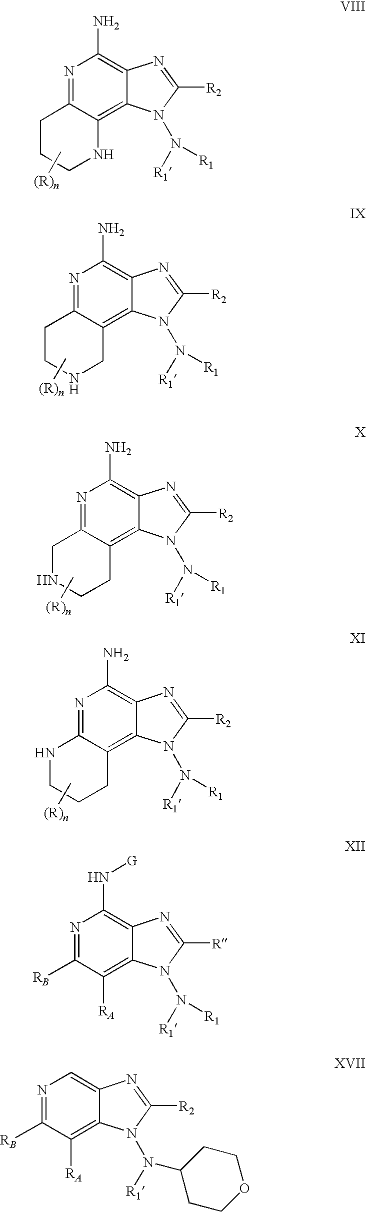

wherein: R₁′, R₁, R₂, R₃, R″, R, R_{A}, R_{B}, R_{A1}, R_{B1}, G, m and n are as defined below.

In one embodiment, the present invention provides a compound of Formula I:

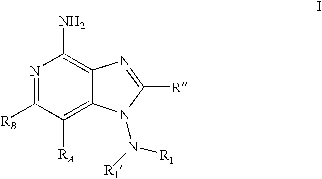

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and

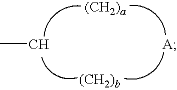

or R₁′ and R₁ together with the nitrogen atom to which they are bonded can join to form a group selected from the group consisting of:

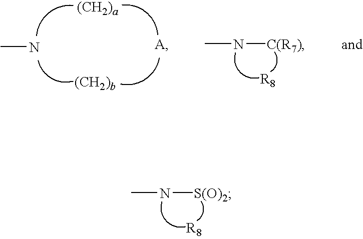

R_{A} and R_{B} are each independently selected from the group consisting of:
hydrogen,
halogen,
alkyl,
alkenyl,
alkoxy,
alkylthio, and
—N(R₁₂)₂;

or when taken together, R_{A} and R_{B} form a fused pyridine ring which is unsubstituted or substituted by one or more R′″ groups;

or when taken together, R_{A} and R_{B} form a fused tetrahydropyridine ring which is unsubstituted or substituted by one or more R groups;

R is selected from the group consisting of:
halogen,
hydroxy,
alkyl,
alkenyl,
haloalkyl,
alkoxy,
alkylthio, and
—N(R₁₂)₂;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

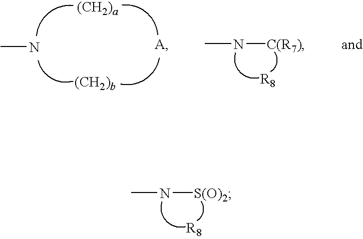

R₆ is selected from the group consisting of hydrogen, alkyl, and arylalkylenyl;

R₇ is selected from the group consisting of ═O and ═S;

R₈ is C₂₋₇ alkylene;

R₁₂ is selected from the group consisting of hydrogen and alkyl;

A is selected from the group consisting of —CH(R₆)-, —O—, —N(R₆)-, —N(Y—R₄)-, and —N(X—N(R₆)—Y—R₄)—;

X is C₂₋₂₀ alkylene;

Y is selected from the group consisting of —C(R₇)—, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

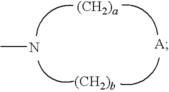

a and b are independently integers from 1 to 4 with the proviso that when

A is —O—, —N(R₆)-, —N(Y—R₄)-, or —N(X—N(R₆)—Y—R₄)- then a and b are independently integers from 2 to 4;

R″ hydrogen or a non-interfering substituent; and

R′″ is a non-interfering substituent;

or a pharmaceutically acceptable salt thereof.

In one embodiment, the present invention provides a compound of Formula II:

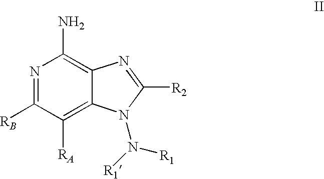

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and

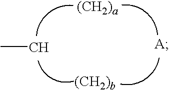

or R₁′ and R₁ together with the nitrogen atom to which they are bonded can join to form a group selected from the group consisting of:

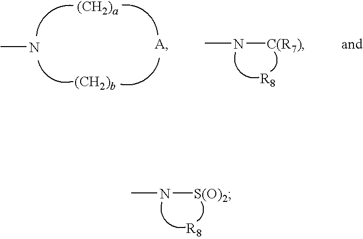

R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
   hydroxy,
   halogen,
   —N(R₆)₂,
   —C(R₇)—N(R₆)₂,
   —S(O)₂—N(R₆)₂,
   —N(R₆)—C(R₇)—C₁₋₁₀ alkyl,
   —N(R₆)—C(R₇)-aryl,
   —N(R₆)—S(O)₂—C₁₋₁₀ alkyl,
   —N(R₆)—S(O)₂-aryl,
   —C(O)—C₁₋₁₀ alkyl,
   —C(O)—O—C₁₋₁₀-alkyl,
   —O—C(R₇)—C₁₋₁₀ alkyl,
   —O—C(R₇)-aryl,
   —O—C(R₇)—N(R₆)—C₁₋₁₀ alkyl,
   —O—C(R₇)—N(R₆)-aryl,
   —N₃,
   aryl,
   heteroaryl,
   heterocyclyl,
   —C(O)-aryl, and
   —C(O)-heteroaryl;

R_{A} and R_{B} are each independently selected from the group consisting of:
hydrogen,
halogen,
alkyl,
alkenyl,
alkoxy,
alkylthio, and
—N(R₁₂)₂;

or when taken together, R_{A} and R_{B} form a fused pyridine ring which is unsubstituted or substituted by one or more R groups, or substituted by one R₃ group, or substituted by one R₃ group and one R group, or substituted by one R₃ group and two R groups;

or when taken together, R_{A} and R_{B} form a fused tetrahydropyridine ring which is unsubstituted or substituted by one or more R groups;

R is selected from the group consisting of:
halogen,
hydroxy,
alkyl,
alkenyl,
haloalkyl,
alkoxy,
alkylthio, and
—N(R₁₂)₂;

R₃ is selected from the group consisting of:
-Z′-R₄′,
-Z′-X′-R₄′,
-Z′-X′—Y—R′, and
-Z′-X′-R₅′;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

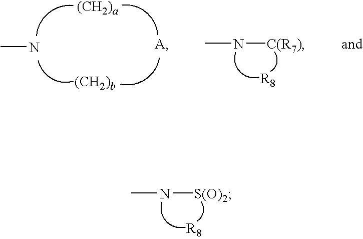

X is C₂₋₂₀ alkylene;

Y is selected from the group consisting of —C(R₇)—, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

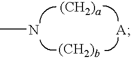

Z is selected from the group consisting of —O— and —S(O)₀₋₂—;

A is selected from the group consisting of —CH(R₆)-, —O—, —N(R₄)-, —N(Y—R₄)-, and —N(X—N(R)—Y—R₄)-;

a and b are independently integers from 1 to 4 with the proviso that when

A is —O—, —N(R₆)-, —N(Y—R₄)-, or —N(X—N(R₆)—Y—R₄)- then a and b are independently integers from 2 to 4;

R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅′ is selected from the group consisting of:

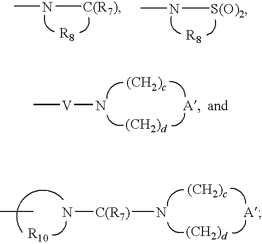

X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups;

Y′ is selected from the group consisting of:
—S(O)₀₋₂—,
—S(O)₂—N(R₁₁)-,
—C(R₇)-,
—C(R₇)—O—,
—O—C(R₇)-,
—O—C(O)—O—,
—N(R₁₁)-Q-,
—C(R₇)—N(R₁₁)-,
—O—C(R₇)—N(R₁₁)-,
—C(R₇)—N(OR₁₂)-,

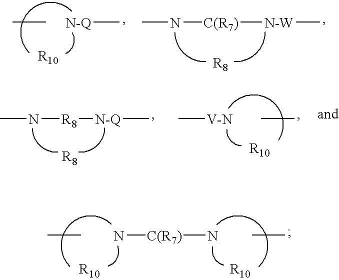

Z′ is a bond or —O—;

A′ is selected from the group consisting of —CH₂—, —O—, —C(O)—, —S(O)₀₋₂—, and —N(R₄′)-;

Q is selected from the group consisting of a bond, —C(R₇)—, —C(R₇)—C(R₇)-, —S(O)₂—, —C(R₇)—N(R₁₁)—W—, —S(O)₂—N(R₁₁)-, —C(R₇)—O—, and —C(R₇)—N(OR₁₂)—;

V is selected from the group consisting of —C(R₇)-, —O—C(R₇)-, —N(R₁₁)—C(R₇)-, and —S(O)₂—;

W is selected from the group consisting of a bond, —C(O)—, and —S(O)₂—;

c and d are independently integers from 1 to 6 with the proviso that c+d is ≦7, and when A′ is —O— or —N(R₄′)- then c and d are independently integers from 2 to 4;

R₆ is selected from the group consisting of hydrogen, alkyl, and arylalkylenyl;

R₇ is selected from the group consisting of ═O and ═S;

R₈ is C₂₋₇ alkylene;

R₁₀ is C₃₋₈ alkylene;

R₁₁ is selected from the group consisting of hydrogen, C₁₋₁₀ alkyl, C₂₋₁₀ alkenyl, C₁₋₁₀ alkoxyC₂₋₁₀ alkylenyl, and arylC₁₋₁₀ alkylenyl; and

R₁₂ is selected from the group consisting of hydrogen and alkyl;

or a pharmaceutically acceptable salt thereof.

In one embodiment, the present invention provides a compound of Formula III:

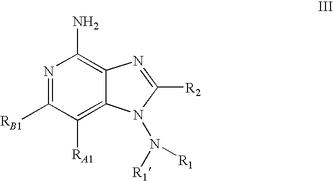

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₄)-R₄,
-X—O—R₄,
-X—S(O)₂—R₄, and

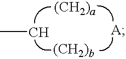

or R₁′ and R₁ together with the nitrogen atom to which they are bonded can join to form a group selected from the group consisting of:

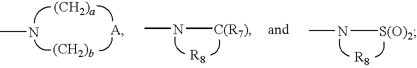

R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
   hydroxy,
   halogen,
   —N(R₆)₂,
   —C(R₇)—N(R₄)₂,
   —S(O)₂—N(R₄)₂,
   —N(R₆)—C(R₇)—C₁₋₁₀ alkyl,
   —N(R₆)—C(R₇)-aryl,
   —N(R₆)—S(O)₂—C₁₋₁₀ alkyl,
   —N(R₆)—S(O)₂-aryl,
   —C(O)—C₁₋₁₀ alkyl,
   —C(O)—O—C₁₋₁₀ alkyl,
   —O—C(R₇)—C₁₋₁₀ alkyl,
   —O—C(R₇)-aryl,
   —O—C(R₇)—N(R₆)—C₁₋₁₀ alkyl,
   —O—C(R₇)—N(R₆)-aryl,
   —N₃,
   aryl,
   heteroaryl,
   heterocyclyl,
   —C(O)-aryl, and
   —C(O)-heteroaryl;

R_{A1} and R_{B1} are each independently selected from the group consisting of:
hydrogen,
halogen,
alkyl,
alkenyl,
alkoxy,
alkylthio, and
—N(R₁₂)₂;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

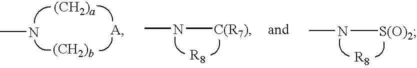

R₆ is selected from the group consisting of hydrogen, alkyl, and arylalkylenyl;

R₇ is selected from the group consisting of ═O and ═S;

R₈ is C₂₋₇ alkylene;

R₁₂ is selected from the group consisting of hydrogen and alkyl;

A is selected from the group consisting of —CH(R₆)-, —O—, —N(R₆)-, —N(Y—R₄)-, and —N(X—N(R₆)—Y—R₄)-;

X is C₂₋₂₀ alkylene;

Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

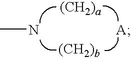

Z is selected from the group consisting of —O— and —S(O)₀₋₂—; and

a and b are independently integers from 1 to 4 with the proviso that when

A is —O—, —N(R₆)-, —N(Y—R₄)-, or —N(X—N(R₆)—Y—R₄)- then a and b are independently integers from 2 to 4;
or a pharmaceutically acceptable salt thereof.

In some embodiments, the present invention provides a compound selected from the group consisting of the following Formulas IV, V, VI, and VII (preferably, a compound of Formula IV):

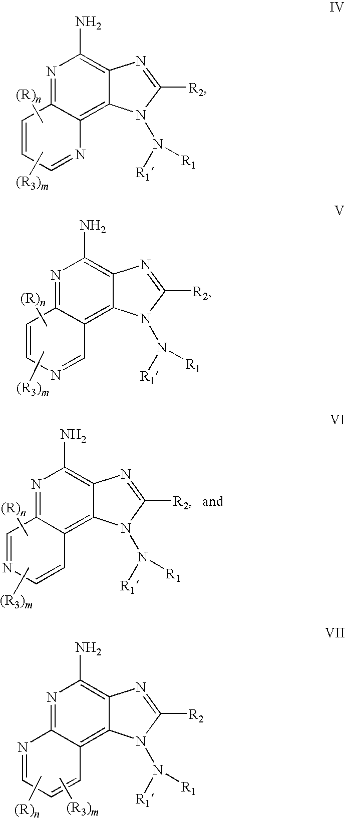

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and

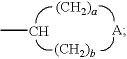

or R₁′ and R₁ together with the nitrogen atom to which they are bonded can join to form a group selected from the group consisting of:

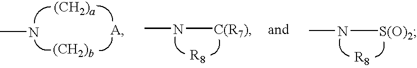

R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
   hydroxy,
   halogen,
   —N(R₆)₂,
   —C(R₇)—N(R₆)₂,
   —S(O)₂—N(R₆)₂,
   —N(R₆)—C(R₇)—C₁₋₁₀ alkyl,
   —N(R₆)—C(R₇)-aryl,
   —N(R₆)—S(O)₂—C₁₋₁₀ alkyl,
   —N(R₆)—S(O)₂-aryl,
   —C(O)—C₁₋₁₀-alkyl,
   —C(O)—O—C₁₋₁₀ alkyl,
   O—C(R₇)—C₁₋₁₀ alkyl,
   —O—C(R₇)-aryl,
   —O—C(R₇)—N(R₆)—C₁₋₁₀ alkyl,
   —O—C(R₇)—N(R₆)-aryl,
   —N₃,
   aryl,
   heteroaryl,
   heterocyclyl,
   —C(O)-aryl, and
   —C(O)-heteroaryl;

R is selected from the group consisting of:
halogen,
hydroxy,
alkyl,
alkenyl,
haloalkyl,
alkoxy,
alkylthio, and
—N(R₁₂)₂;

R₃ is selected from the group consisting of:
-Z′-R₄′,
-Z′-x′-R₄′,
-Z′-X′—Y′—R₄′, and
-Z′-X′-R_{5′;}

n is an integer from 0 to 3;

m is 0 or 1, with the proviso that when m is 1, n is 0, 1, or 2;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

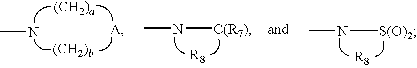

X is C₂₋₂₀ alkylene;

Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

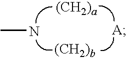

Z is selected from the group consisting of —O— and —S(O)₀₋₂—;

A is selected from the group consisting of —CH(R₆)-, —O—, —N(R₆)-, —N(Y—R₄)-, and —N(X—N(R₆)—Y—R₄)-;

a and b are independently integers from 1 to 4 with the proviso that when A is —O—, —N(R₆)-, —N(Y—R₄)-, or —N(X—N(R₆)—Y—R₄)- then a and b are independently integers from 2 to 4;

R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅′ is selected from the group consisting of:

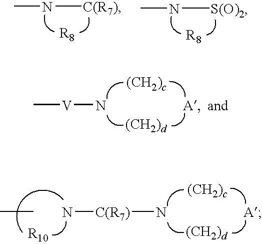

X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups;

Y′ is selected from the group consisting of:
—S(O)₀₋₂—,
—S(O)₂—N(R₁₁)-,
—C(R₇)-,
—C(R₇)—O—,
—O—C(R₇)—,
—O—C(O)—O—,
—N(R₁₁)-Q-,
—C(R₇)—N(R₁₁)-,
—O—C(R₇)—N(R₁₁)-,
—C(R₇)—N(OR₁₂)-,

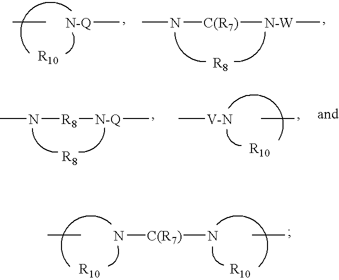

Z′ is a bond or —O—;

A′ is selected from the group consisting of —CH₂—, —O—, —C(O)—, —S(O)₀₋₂—, and —N(R₄′)-;

Q is selected from the group consisting of a bond, —C(R₇)-, —C(R₇)—C(R₇)-, —S(O)₂—, —C(R₇)—N(R₁₁)—W—, —S(O)₂—N(R₁₁)-, —C(R₇)—O—, and —C(R₇)—N(OR₁₂)-;

V is selected from the group consisting of —C(R₇)-, —O—C(R₇)-, —N(R₁₁)—C(R₇)-, and —S(O)₂—;

W is selected from the group consisting of a bond, —C(O)—, and —S(O)₂—;

c and d are independently integers from 1 to 6 with the proviso that c+d is ≦7, and when A′ is —O— or —N(R₄′)- then c and d are independently integers from 2 to 4;

R₆ is selected from the group consisting of hydrogen, alkyl, and arylalkylenyl;

R₇ is selected from the group consisting of ═O and ═S;

R₈ is C₂₋₇ alkylene;

R₁₀ is C₃₋₈ alkylene;

R₁₁ is selected from the group consisting of hydrogen, C₁₋₁₀ alkyl, C₂₋₁₀ alkenyl, C₁₋₁₀ alkoxyC₂₋₁₀ alkylenyl, and arylC₁₋₁₀ alkylenyl; and

R₁₂ is selected from the group consisting of hydrogen and alkyl;

or a pharmaceutically acceptable salt thereof.

In some embodiments, the present invention provides a compound selected from the group consisting of the following Formulas VIII, IX, X, and XI (preferably, a compound of Formula VIII:

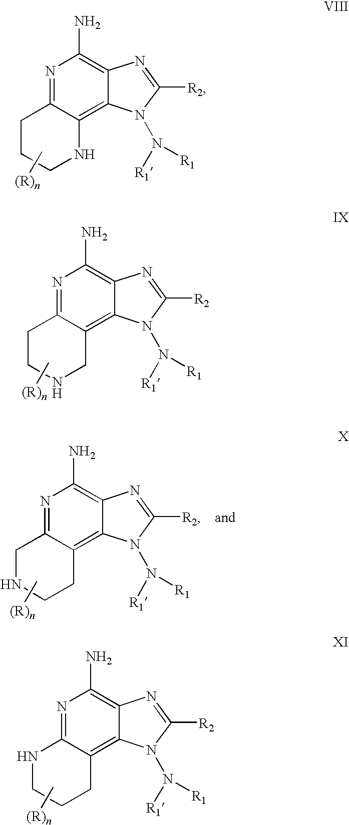

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and

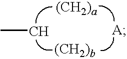

or R₁′ and R₁ together with the nitrogen atom to which they are bonded can join to form a group selected from the group consisting of:

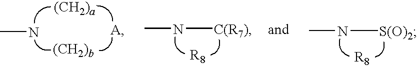

R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
   hydroxy,
   halogen,
   —N(R)₂,
   —C(R₇)—N(R₆)₂,
   —S(O)₂—N(R₆)₂,
   —N(R₆)—C(R₇)—C₁₋₁₀ alkyl,
   —N(R₆)—C(R₇)-aryl,
   —N(R₆)—S(O)₂—C₁₋₁₀ alkyl,
   —N(R₆)—S(O)₂-aryl,
   —C(O)—C₁₋₁₀ alkyl,
   —C(O)—O—C₁₋₁₀ alkyl,
   —O—C(R₇)—C₁₋₁₀ alkyl,
   —O—C(R₇)-aryl,
   —O—C(R₇)—N(R₆)—C₁₋₁₀ alkyl,
   —O—C(R₇)—N(R₆)-aryl,
   —N₃,
   aryl,
   heteroaryl,
   heterocyclyl,
   —C(O)-aryl, and
   —C(O)-heteroaryl;

R is selected from the group consisting of:
halogen,
hydroxy,
alkyl,
alkenyl,
haloalkyl,
alkoxy,
alkylthio, and
—N(R₁₂)₂;

n is an integer from 0 to 3;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

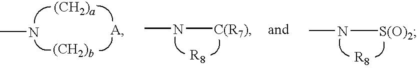

R₆ is selected from the group consisting of hydrogen, alkyl, and arylalkylenyl;

R₇ is selected from the group consisting of ═O and ═S;

R₈ is C₂₋₇ alkylene;

R₁₂ is selected from the group consisting of hydrogen and alkyl;

A is selected from the group consisting of —CH(R₆)-, —O—, —N(R₆)-, —N(Y—R₄)-, and —N(X—N(R₆)—Y—R₄)-;

X is C₂₋₂₀ alkylene;

Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

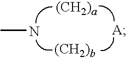

Z is selected from the group consisting of —O— and —S(O)₀₋₂—; and

a and b are independently integers from 1 to 4 with the proviso that when

A is —O—, —N(R₆)-, —N(Y—R₄)-, or —N(X—N(R₆)—Y—R₄)- then a and b are independently integers from 2 to 4;
or a pharmaceutically acceptable salt thereof.

For certain embodiments of the compounds of Formulas I through XI, the —NH₂ group can be replaced by an —NH-G group, as shown in the compound of Formula XII, to form prodrugs. In such embodiments, G is selected from the group consisting of: —C(O)—R′, α-aminoacyl, α-aminoacyl-α-aminoacyl, —C(O)—O—R′, —C(O)—N(R″″)-R′, —C(═NY₂)—R′, —CH(OH)—C(O)—OY₂, —CH(OC₁₋₄ alkyl)Y₀, —CH₂Y₁, and —CH(CH₃)Y₁; wherein R′ and R″″ are each independently C₁₋₁₀ alkyl, C₃₋₇ cycloalkyl, phenyl, or benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂; α-aminoacyl is an acyl group derived from an amino acid selected from the group consisting of racemic, D-, and L-amino acids; Y₂ is selected from the group consisting of hydrogen, C₁₋₆ alkyl, and benzyl; Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl; Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl.

For example, the present invention provides a compound of the Formula XII:

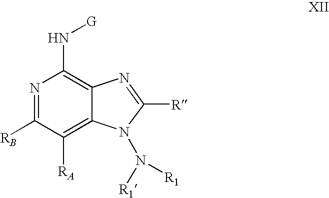

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and

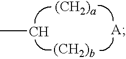

or R₁′ and R₁ together with the nitrogen atom to which they are bonded can join to form a group selected from the group consisting of:

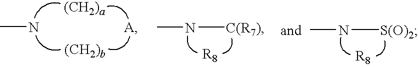

R_{A} and R_{B} are each independently selected from the group consisting of:
hydrogen,
halogen,
alkyl,
alkenyl,
alkoxy,
alkylthio, and
—N(R₁₂)₂;

or when taken together, R_{A} and R_{B} form a fused pyridine ring which is unsubstituted or substituted by one or more R′″ groups;

or when taken together, R_{A} and R_{B} form a fused tetrahydropyridine ring which is unsubstituted or substituted by one or more R groups;

R is selected from the group consisting of:
halogen,
hydroxy,
alkyl,
alkenyl,
haloalkyl,
alkoxy,
alkylthio, and
—N(R₁₂)₂;

R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;

R₅ is selected from the group consisting of:

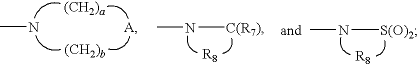

R₆ is selected from the group consisting of hydrogen, alkyl, and arylalkylenyl;

R₇ is selected from the group consisting of ═O and ═S;

R₈ is C₂₋₇ alkylene;

R₁₂ is selected from the group consisting of hydrogen and alkyl;

A is selected from the group consisting of —CH(R₆)-, —O—, —N(R₆)-, —N(Y—R₄)-, and —N(X—N(R₆)—Y—R₄)-;

X is C₂₋₂₀ alkylene;

Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

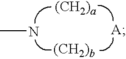

a and b are independently integers from 1 to 4 with the proviso that when

A is —O—, —N(R₆)-, —N(Y—R₄)-, or —N(X—N(R₆)—Y—R₄)- then a and b are independently integers from 2 to 4;

R″ hydrogen or a non-interfering substituent;

R′″ is a non-interfering substituent;

G is selected from the group consisting of:
—C(O)—R′,
α-aminoacyl,
α-aminoacyl-α-aminoacyl,
—C(O)—O—R′,
—C(O)—N(R″″)-R′,
—C(═NY₂)—R′,
—CH(OH)—C(O)—OY₂,
—CH(OC₁₋₄ alkyl)Y₀,
—CH₂Y₁, and
—CH(CH₃)Y₁;

R′ and R″″ are each independently C₁₋₁₀ alkyl, C₃₋₇ cycloalkyl, phenyl, or benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂;

α-aminoacyl is an acyl group derived from an amino acid selected from the group consisting of racemic, D-, and L-amino acids;

Y₂ is selected from the group consisting of hydrogen, C₁₋₆ alkyl, and benzyl;

Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₄ alkylaminoC₁₋₄ alkylenyl;

Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl;

or a pharmaceutically acceptable salt thereof.

In some embodiments, the present invention provides a compound of the following Formula XVII:

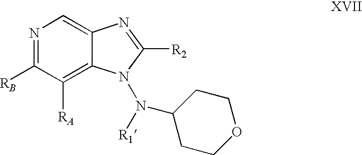

wherein:

R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;

R₂ is selected from the group consisting of:
hydrogen,
alkyl
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
   hydroxy,
   halogen,
   —N(R)₂,
   —C(R₇)—N(R₆)₂,
   —S(O)₂—N(R₆)₂,
   —N(R₆)—C(R₇)—C₁₋₁₀ alkyl,
   —N(R₆)—C(R₇)-aryl,
   —N(R₆)—S(O)₂—C₁₋₁₀ alkyl,
   —N(R₆)—S(O)₂-aryl,
   —C(O)—C₁₋₁₀ alkyl,
   —C(O)—O—C₁₋₁₀ alkyl,
   —O—C(R₇)—C₁₋₁₀ alkyl,
   —O—C(R₇)-aryl,
   —O—C(R₇)—N(R₆)—C₁₋₁₀ alkyl,
   —O—C(R₇)—N(R₆)-aryl,
   —N₃,
   aryl,
   heteroaryl,
   heterocyclyl,
   —C(O)-aryl, and
   —C(O)-heteroaryl;

R_{A} and R_{B} are each independently selected from the group consisting of:
hydrogen,
halogen,
alkyl,
alkenyl,
alkoxy,
alkylthio, and
—N(R₁₂)₂;

or when taken together, R_{A} and R_{B} form a fused pyridine ring which is unsubstituted or substituted by one or more R groups, or substituted by one R₃ group, or substituted by one R₃ group and one R group, or substituted by one R₃ group and two R groups;

or when taken together, R_{A} and R_{B} form a fused tetrahydropyridine ring which is unsubstituted or substituted by one or more R groups;

R is selected from the group consisting of:
halogen,
hydroxy,
alkyl,
alkenyl,
haloalkyl,
alkoxy,
alkylthio, and
—N(R₁₂)₂;

R₃ is selected from the group consisting of:
-Z′-R₄′,
-Z′-X′-R₄′,
-Z′-X′—Y′—R₄′, and
-Z′-X′-R₅′;

Z is selected from the group consisting of —O— and —S(O)₀₋₂—;

R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅′ is selected from the group consisting of:

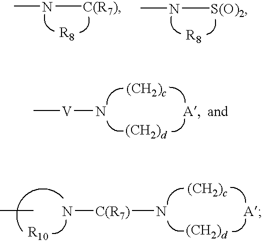

X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups;

Y′ is selected from the group consisting of:
—S(O)₀₋₂—,
—S(O)₂—N(R₁₁)-,
—C(R₇)—,
—C(R₇)—O—,
—O—C(R₇)-,
—O—C(O)—O—,
—N(R₁₁)-Q-,
—C(R₇)—N(R₁₁)-,
—O—C(R₇)—N(R₁₁)-,
—C(R₇)—N(OR₁₂)-,

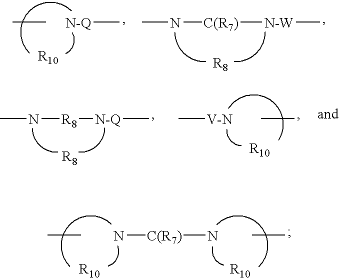

Z′ is a bond or —O—;

A′ is selected from the group consisting of —CH₂—, —O—, —C(O)—, —S(O)₀₋₂—, and —N(R₄′)-;

Q is selected from the group consisting of a bond, —C(R₇)-, —C(R₇)—C(R₇)-, —S(O)₂—, —C(R₇)—N(R₁₁)—W—, —S(O)₂—N(R₁₁)-, —C(R₇)—O—, and —C(R₇)—N(OR₁₂)-;

V is selected from the group consisting of —C(R₇)-, —O—C(R₇)-, —N(R₁₁)—C(R₇)-, and —S(O)₂—;

W is selected from the group consisting of a bond, —C(O)—, and —S(O)₂—;

c and d are independently integers from 1 to 6 with the proviso that c+d is ≦7, and when A′ is —O— or —N(R₄′)- then c and d are independently integers from 2 to 4;

R₆ is selected from the group consisting of hydrogen, alkyl, and arylalkylenyl;

R₇ is selected from the group consisting of ═O and ═S;

R₈ is C₂₋₇ alkylene;

R₁₀ is C₃₋₈ alkylene;

R₁₁ is selected from the group consisting of hydrogen, C₁₋₁₀ alkyl, C₂₋₁₀ alkenyl, C₁₋₁₀ alkoxyC₂₋₁₀ alkylenyl, and arylC₁₋₁₀ alkylenyl; and

R₁₂ is selected from the group consisting of hydrogen and alkyl;

or a pharmaceutically acceptable salt thereof.

For any of the compounds presented herein, each one of the following variables (e.g., R, R″, R′″, R₁′, R₁, R₂, R₃, R_{A}, R_{B}, R_{A1}, R_{B1}, m, n, A, and so on) in any of its embodiments can be combined with any one or more of the other variables in any of their embodiments as would be understood by one of skill in the art. Each of the resulting combinations of variables is an embodiment of the present invention.

For certain embodiments, each of R″ and R′″ is independently a non-interfering substituent. For certain embodiments, each R″ is independently selected from the group consisting of hydrogen and non-interfering substituents. Herein, “non-interfering” means that the immunomodulator activity (for example, the ability to induce the biosynthesis of one or more cytokines) of the compound, which contains the non-interfering substituent, is not destroyed. Illustrative R″ groups include those described herein for R₂. Illustrative R′″ groups include those described herein for R and R₃.

For certain embodiments, R′ and R″″ are each independently C₁₋₁₀ alkyl, C₃₋₇ cycloalkyl, phenyl, or benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂.

For certain embodiments, R is selected from the group consisting of halogen, hydroxy, alkyl, alkenyl, haloalkyl, alkoxy, alkylthio, and —N(R₁₂)₂.

For certain embodiments, R₁′ is selected from the group consisting of: hydrogen, alkyl, hydroxyalkyl, and alkoxyalkyl wherein the alkyl group contains at least 2 carbon atoms between the hydroxy or alkoxy substituent and the nitrogen atom to which R₁′ is bonded. For certain embodiments, R₁′ is hydrogen or alkyl. For certain embodiments, R₁′ is hydrogen or methyl. For certain embodiments, R₁′ is hydrogen.

For certain embodiments, R₁ is selected from the group consisting of:

-R₄, —Y—R₄, -X-R₅, -X—N(R₆)—Y—R₄, -X—C(R₇)—N(R₆)-R₄, -X—O—C(R₇)—N(R₆)-R₄, -X—S(O)₂—N(R₆)-R₄, -X—O—R₄, -X—S(O)₂—R₄, and

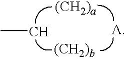

For certain embodiments, R₁ is selected from the group consisting of -R₄, —Y—R₄, -X-R₅, -X—N(R₆)—Y—R₄, -X—C(R₇)—N(R₆)-R₄, -X—O—C(R₇)—N(R₆)-R₄, -X—S(O)₂—N(R₆)-R₄, and -X—O—R₄.

For certain embodiments, R₁ is selected from the group consisting of:

-R₄, —Y—R₄, -X-R₅, -X—N(R₆)—Y—R₄, -X—C(R₇)—N(R₆)-R₄, -X—O—C(R₇)—N(R₆)-R₄, -X—S(O)₂—N(R₆)-R₄, and -X—O—R₄; or R₁′ and R₁ together with the nitrogen atom to which they are bonded can join to form a group selected from the group consisting of:

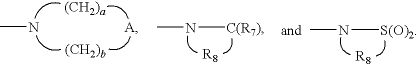

For certain embodiments, R₁ is selected from the group consisting of -R₄ and -X—N(R₆)—Y—R₄. For certain embodiments, R₁ is -R₄. For certain embodiments, R₁ is selected from the group consisting of: isopropyl, cyclohexyl, benzyl, 3-phenylpropyl, and (pyridin-3-yl)methyl. For certain embodiments, -R₁ is C₂₋₆ alkyl. For certain of these embodiments, R₁ is isopropyl or cyclohexyl. For certain of these embodiments, R₁ is isopropyl.

For certain embodiments, R₁ is -X—N(R₆)—Y—R₄. For certain of these embodiments, X is C₂₋₄ alkylene; R₆ is hydrogen or C₁₋₄ alkyl; Y is selected from the group consisting of —C(O)—, —S(O)₂—, and —C(O)—NH—; R₄ is C₁₋₆ alkyl, phenyl, or pyridyl wherein the phenyl or pyridyl groups are optionally substituted with one or more substituents independently selected from the group consisting of alkyl, alkoxy, hydroxy, halogen, cyano, and alkylamino; or —Y—R₄ is

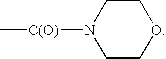

For certain embodiments, R₁ is

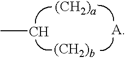

For certain of these embodiments, R₁ is selected from the group consisting of 3-[(methanesulfonyl)amino]propyl, 3-(acetylamino)propyl, 3-[(isopropylcarbonyl)amino]propyl, 3-[(cyclohexylcarbonyl)amino]propyl, 3-[(morpholin-4-ylcarbonyl)amino]propyl, and 3-{[(isopropylamino)carbonyl]amino}propyl.

For certain embodiments, R₁ is -X-R₅. For certain of these embodiments, X is C₂₋₄ alkylene; and R₅ is

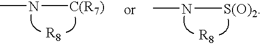

For certain embodiments, R₁ is selected from the group consisting of isopropyl, cyclohexyl, benzyl, 3-phenylpropyl, (pyridin-3-yl)methyl, 3-[(methanesulfonyl)amino]propyl, 3-(acetylamino)propyl, 3-[(isopropylcarbonyl)amino]propyl, 3-[(cyclohexylcarbonyl)amino]propyl, 3-[(morpholin-4-ylcarbonyl)amino]propyl, 3-{[(isopropylamino)carbonyl]amino}propyl, tetrahydropyran-4-yl, methyl, cyclobutyl, 2-(methylsulfonyl)ethyl, 3-(methylsulfonyl)propyl, 2-[(methanesulfonyl)amino]ethyl, 4-[(methanesulfonyl)amino]butyl, 3,4-dichlorobenzyl, (2-fluoropyridin-3-yl)methyl, 1-(methylsulfonyl)piperidin-4-yl, 1-acetylpiperidin-4-yl, 3-[(ethoxycarbonyl)amino]propyl, cyclopentyl, and 3-[(isopropoxycarbonyl)amino]propyl.

For certain embodiments, R₁ is selected from the group consisting of isopropyl, cyclohexyl, benzyl, (pyridin-3-yl)methyl, 3-[(methanesulfonyl)amino]propyl, 3-{[(isopropylamino)carbonyl]amino}propyl, tetrahydropyran-4-yl, methyl, 1-(methylsulfonyl)piperidin-4-yl, 1-acetylpiperidin-4-yl, 3-[(ethoxycarbonyl)amino]propyl, cyclopentyl, 3-[(cyclohexylcarbonyl)amino]propyl, 3-(methylsulfonyl)propyl, 3,4-dichlorobenzyl, and cyclobutyl.

For certain embodiments, R₁ is selected from the group consisting of: isopropyl, cyclohexyl, benzyl, 3-phenylpropyl, (pyridin-3-yl)methyl, 3-[(methanesulfonyl)amino]propyl, 3-(acetylamino)propyl, 3-[(isopropylcarbonyl)amino]propyl, 3-[(cyclohexylcarbonyl)amino]propyl, 3-[(morpholin-4-ylcarbonyl)amino]propyl, and 3-{[(isopropylamino)carbonyl]amino}propyl.

For certain embodiments, R₁ is selected from the group consisting of: isopropyl, cyclohexyl, benzyl, 3-phenylpropyl, (pyridin-3-yl)methyl, 3-[(methanesulfonyl)amino]propyl, 3-(acetylamino)propyl, 3-[(isopropylcarbonyl)amino]propyl, 3-[(morpholin-4-ylcarbonyl)amino]propyl, 3-{[(isopropylamino)carbonyl]amino}propyl, tetrahydropyran-4-yl, 3-(methylsulfonyl)propyl, 2-(methylsulfonyl)ethyl, 1-(methylsulfonyl)piperidin-4-yl, 1-acetylpiperidin-yl, 1-(isopropylcarbonyl)piperidin-4-yl, 1-(morpholin-4-ylcarbonyl)piperidinyl, 1-[(isopropylamino)carbonyl]piperidin-4-yl, cyclobutyl, cyclopentyl, and 2-[(methanesulfonyl)amino]ethyl.

For certain embodiments, R₁ is isopropyl.

For certain embodiments, R₁′ and R₁ together with the nitrogen atom to which they are bonded can join to form a group selected from the group consisting of

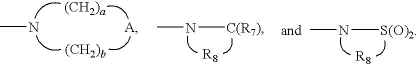

For certain embodiments, R_{A} and R_{B} are each independently selected from the group consisting of hydrogen, halogen, alkyl, alkenyl, alkoxy, alkylthio, and —N(R₁₂)₂; or when taken together, R_{A} and R_{B} form a fused pyridine ring which is unsubstituted or substituted by one or more R′″ groups; or when taken together, R_{A} and R_{B} form a fused tetrahydropyridine ring which is unsubstituted or substituted by one or more R groups. In the fused tetrahydropyridine ring the unsaturated carbon atoms are those in common with the pyridine ring.

For certain embodiments, R_{A} and R_{B} are each independently selected from the group consisting of hydrogen, halogen, alkyl, alkenyl, alkoxy, alkylthio, and —N(R₁₂)₂; or when taken together, R_{A} and R_{B} form a fused pyridine ring which is unsubstituted or substituted by one or more R groups, or substituted by one R₃ group, or substituted by one R₃ group and one R group, or substituted by one R₃ group and two R groups; or when taken together, R_{A} and R_{B} form a fused tetrahydropyridine ring which is unsubstituted or substituted by one or more R groups.

For certain embodiments, R_{A} and R_{B} form a fused pyridine ring which is unsubstituted or substituted by one or more R groups, or substituted by one R₃ group, or substituted by one R₃ group and on R group, or substituted by one R₃ group and two R groups.

For certain embodiments, R_{A} and R_{B} are each independently selected from the group consisting of: hydrogen, halogen, alkyl, alkenyl, alkoxy, alkylthio, and —N(R₁₂)₂. For certain embodiments, R_{A} and R_{B} are each independently selected from hydrogen and alkyl. For certain embodiments, R_{A} and R_{B} are each methyl.

For certain embodiments, R_{A} and R_{B} form a fused pyridine ring which is unsubstituted or substituted by one or more R′″ groups. For certain of these embodiments, R_{A} and R_{B} form a fused pyridine ring wherein the fused pyridine ring is

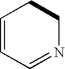

wherein the highlighted bond indicates the position where the ring is fused.

For certain embodiments, R_{A} and R_{B} form a fused tetrahydropyridine ring which is unsubstituted or substituted by one or more R groups. For certain of these embodiments, R_{A} and R_{B} form a fused tetrahydropyridine ring wherein the fused tetrahydropyridine ring is

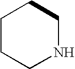

wherein the highlighted bond indicates the position where the ring is fused.

For certain embodiments, R_{A1} and R_{B1} are each independently selected from the group consisting of hydrogen, halogen, alkyl, alkenyl, alkoxy, alkylthio, and —N(R₁₂)₂.

For certain embodiments, R_{A1} and R_{B}, are each independently selected from hydrogen and alkyl. For certain of these embodiments, R_{A1} and R_{B1} are each methyl.

For certain embodiments, R₂ is selected from the group consisting of: hydrogen, alkyl, alkenyl, aryl, heteroaryl, heterocyclyl, alkyl-Z-alkylenyl, aryl-Z-alkylenyl, alkenyl-Z-alkylenyl, and alkyl or alkenyl substituted by one or more substituents selected from the group consisting of hydroxy, halogen, —N(R₆)₂, —C(R₇)—N(R₆)₂, —S(O)₂—N(R₆)₂, —N(R₆)—C(R₇)—C₁₋₁₀ alkyl, —N(R₆)—C(R₇)-aryl, —N(R₆)—S(O)₂—C₁₋₁₀ alkyl, —N(R₆)—S(O)₂-aryl, —C(O)—C₁₋₁₀ alkyl, —C(O)—O—C₁₋₁₀ alkyl, —O—C(R₇)—C₁₋₁₀ alkyl, —O—C(R₇)-aryl, —O—C(R₇)—N(R₆)—C₁₋₁₀ alkyl, —O—C(R₇)—N(R₆)-aryl, —N₃, aryl, heteroaryl, heterocyclyl, —C(O)-aryl, and —C(O)-heteroaryl.

For certain embodiments, R₂ is selected from the group consisting of hydrogen, alkyl, hydroxyalkyl, and alkoxyalkylenyl. For certain embodiments, R₂ is selected from the group consisting of hydrogen, methyl, ethyl, n-propyl, n-butyl, methoxymethyl, ethoxymethyl, 2-methoxyethyl, hydroxymethyl, 2-hydroxyethyl, and 3-hydroxypropyl.

For certain embodiments, R₂ is selected from the group consisting of hydrogen, methyl, ethyl, n-propyl, n-butyl, methoxymethyl, ethoxymethyl, 2-methoxyethyl, hydroxymethyl, and 2-hydroxyethyl.

For certain embodiments, R₃ is selected from the group consisting of -Z′-R₄′, -Z′-X′-R₄′, -Z′-X′—Y′—R₄′, and -Z′-X′-R₅′.

For certain embodiments, R₃ is selected from the group consisting of -Z′-R₄′, and -Z′-X′—Y—R₄′.

For certain embodiments, R₃ is -Z-R₄′. For certain of these embodiments, Z′ is a bond, and R₄′ is phenyl or pyridyl.

For certain embodiments, R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded.

For certain embodiments, R₄ is selected from the group consisting of hydrogen, alkyl, alkenyl, alkynyl, aryl, heteroaryl, and heterocyclyl wherein the alkyl, alkenyl, alkynyl, aryl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded.

For certain embodiments, R₄ is selected from the group consisting of C₁₋₆ alkyl, phenyl, or pyridyl wherein the phenyl or pyridyl groups are optionally substituted with one or more substituents independently selected from the group consisting of alkyl, alkoxy, hydroxy, halogen, cyano, and alkylamino. For certain embodiments, R₄ is C₂₋₆ alkyl.

For certain embodiments, R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo.

For certain embodiments, R₄′ is phenyl or pyridyl.

For certain embodiments, R₅ is selected from the group consisting of

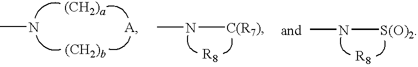

For certain embodiments, R₅ is

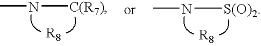

For certain embodiments, R₅′ is selected from the group consisting of

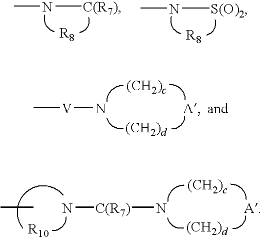

For certain embodiments, R₆ is selected from the group consisting of hydrogen, alkyl, and arylalkylenyl. For certain embodiments, R₆ is hydrogen or C₁₋₄ alkyl.

For certain embodiments, R₇ is selected from the group consisting of ═O and ═S. For certain embodiments, R₇ is ═O.

For certain embodiments, R₈ is C₂₋₇ alkylene.

For certain embodiments, R₉ is selected from the group consisting of hydrogen, alkyl, and arylalkylenyl. For certain embodiments, R₉ and R₄ together with the nitrogen atom to which R₉ is bonded can join to form the group

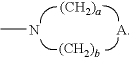

For certain of these embodiments, a and b are independently integers from 1 to 4 with the proviso that when A is —O—, —N(R₆)-, —N(Y—R₄)-, or —N(X—N(R₆)—Y—R₄)- then a and b are independently integers from 2 to 4. For certain of these embodiments, a and b are each the integer 2. For certain of these embodiments, A is —O—.

For certain embodiments, R₁₀ is C₃₋₈ alkylene.

For certain embodiments, R₁₁ is selected from the group consisting of hydrogen, C₁₋₁₀ alkyl, C₂₋₁₀ alkenyl, C₁₋₁₀ alkoxyC₂₋₁₀ alkylenyl, and arylC₁₋₁₀ alkylenyl.

For certain embodiments, R₁₂ is selected from the group consisting of hydrogen and alkyl.

For certain embodiments, A is selected from the group consisting of —CH(R₆)-, —O—, —N(R₆)-, —N(Y—R₄)-, and —N(X—N(R₆)—Y—R₄)-. For certain embodiments, A is —O—, —N(R₆)-, —N(Y—R₄)-, or —N(X—N(R₆)—Y—R₄)-. For certain embodiments, A is —O—.

For certain embodiments, A′ is selected from the group consisting of —CH₂—, —O—, —C(O)—, —S(O)₀₋₂—, and —N(R₄′)-. For certain embodiments, A′ is —O— or —N(R₄′)-.

For certain embodiments, G is selected from the group consisting of —C(O)—R′, α-aminoacyl, α-aminoacyl-α-aminoacyl, —C(O)—O—R′, —C(O)—N(R″″)-R′, —C(═NY₂)—R′, —CH(OH)—C(O)—OY₂, —CH(OC₁₋₄ alkyl)Y₀, —CH₂Y₁, and —CH(CH₃)Y₁. In certain embodiments, α-aminoacyl is an acyl group derived from an amino acid selected from the group consisting of racemic, D-, and L-amino acids.

For certain embodiments, Q is selected from the group consisting of a bond, —C(R₇)-, —C(R₇)—C(R₇)—, —S(O)₂—, —C(R₇)—N(R₁₁)—W—, —S(O)₂—N(R₁₁)-, —C(R₇)—O—, and —C(R₇)—N(OR₁₂)-.

For certain embodiments, V is selected from the group consisting of —C(R₇)-, —O—C(R₇)-, —N(R₁₁)—C(R₇)-, and —S(O)₂—.

For certain embodiments, W is selected from the group consisting of a bond, —C(O)—, and —S(O)₂—.

For certain embodiments, X is C₂₋₂₀ alkylene.

For certain embodiments, X is C₂₋₄ alkylene.

For certain embodiments, X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups.

For certain embodiments, Y is selected from the group consisting of —C(R₇)-, —C(R₇)—O—, —S(O)₂—, —S(O)₂—N(R₆)-, and —C(R₇)—N(R₉)-.

For certain embodiments, Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group

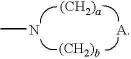

For certain of these embodiments, a and b are each the integer 2. For certain of these embodiments, A is —O—.

For certain embodiments, Y is selected from the group consisting of —C(O)—, —S(O)₂—, and —C(O)—NH—. For certain embodiments, —Y—R₄ is

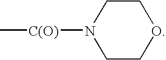

For certain embodiments, Y′ is selected from the group consisting of —S(O)₀₋₂—, —S(O)₂—N(R₁₁)-, —C(R₇)-, —C(R₇)—O—, —O—C(R₇)-, —O—C(O)—O—, —N(R₁₁)-Q-, —C(R₇)—N(R₁₁)-, —O—C(R₇)—N(R₁₁)-, —C(R₇)—N(OR₁₂)-,

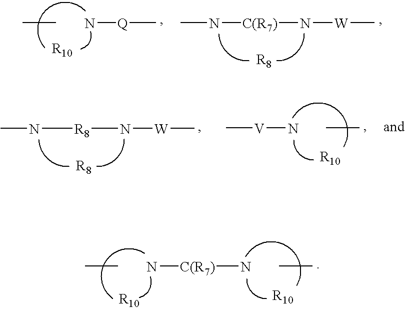

For certain embodiments, Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl.

For certain embodiments, Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl.

For certain embodiments, Y₂ is selected from the group consisting of hydrogen, C₁₋₆ alkyl, and benzyl.

For certain embodiments, Z is selected from the group consisting of —O— and —S(O)₀₋₂—. For certain embodiments, Z is —O—.

For certain embodiments, Z′ is a bond or —O—. For certain embodiments, Z′ is a bond.

For certain embodiments, m is 0 or 1, with the proviso that when m is 1, n is 0, 1, or 2. For certain embodiments, m is 0. For certain embodiments, m is 1. For certain embodiments, m is 0, and n is 0. For certain embodiments, m is 1, and n is 0. For certain embodiments m is 1, and n is 0 or 1. For certain embodiments, m is 1, and n is 1.

For certain embodiments, n is an integer from 0 to 3. For certain embodiments, n is 0, 1, or 2. For certain embodiments, n is 0. For certain embodiments, n is 1.

For certain embodiments, a and b are independently integers from 1 to 4. For certain embodiments, a and b are independently integers from 2 to 4. For certain embodiments, a and b are independently integers from 1 to 4 with the proviso that when A is —O—, —N(R₆)-, —N(Y—R₄)-, or —N(X—N(R₆)—Y—R₄)- then a and b are independently integers from 2 to 4. For certain embodiments, a and b are each the integer 2.

For certain embodiments, c and d are independently integers from 1 to 6. For certain embodiments, c+d is ≦7. For certain embodiments, particularly when A′ is —O— or —N(R₄′)- then c and d are independently integers from 2 to 4.

As used herein, the terms “alkyl”, “alkenyl”, “alkynyl” and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene,” “-alkylene-”, “alkenylene”, “-alkenylene-”, “alkynylene”, and “-alkynylene-” are the divalent forms of the “alkyl”, “alkenyl”, and “alkynyl” groups defined above. The terms “alkylenyl”, “alkenylenyl”, and “alkynylenyl” are used when “alkylene”, “alkenylene”, and “alkynylene”, respectively, are substituted. For example, an arylalkylenyl group comprises an “alkylene” moiety to which an aryl group is attached.

The term “haloalkyl” is inclusive of alkyl groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

The term “heteroatom” refers to the atoms O, S, or N.

The term “heteroaryl”, includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2 to 12 carbon atoms, 1 to 3 rings, 1 to 4 heteroatoms, and O, S, and N as the heteroatoms. Exemplary heterocyclic groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like. The term “heterocyclyl” includes bicyclic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene”, “heteroarylene”, and “heterocyclylene” are the divalent forms of the “aryl”, “heteroaryl”, and “heterocyclyl” groups defined above. The terms “arylenyl,” “heteroarylenyl,” and “heterocyclylenyl” are used when “arylene”, “heteroarylene”, and “heterocyclylene”, respectively, are substituted. For example, an alkylarylenyl group comprises an arylene moiety to which an alkyl group is attached.

When a group (or substituent or variable) is present more than once in any Formula described herein, each group (or substituent or variable) is independently selected, whether explicitly stated or not. For example, for the formula —N(R₁₂)₂ each R₁₂ group is independently selected. In another example, when an R₁, and an R₂ group both contain an R₆ group, each R₆ group is independently selected. In a further example, when more than one

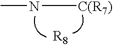

group is present (i.e., R₅ and R₅′ both contain a

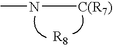

group) each R₈ group is independently selected and each R₇ group is independently selected.

The invention is inclusive of the compounds described herein (including intermediates) in any of their pharmaceutically acceptable forms, including isomers (e.g., diastereomers and enantiomers), salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

The term “prodrug” means a compound that can be transformed in vivo to yield an immune response modifying compound in any of the salt, solvated, polymorphic, or isomeric forms described above. The prodrug, itself, may be an immune response modifying compound in any of the salt, solvated, polymorphic, or isomeric forms described above. The transformation may occur by various mechanisms, such as through a chemical (e.g., solvolysis or hydrolysis, for example, in the blood) or enzymatic biotransformation. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

### Preparation of the Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

In the preparation of compounds of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl, and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, John Wiley & Sons, New York, USA, 1991.

Conventional methods and techniques of separation and purification can be used to isolate compounds of the invention, as well as various, pharmaceutically acceptable salts thereof and intermediates related thereto. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

Compounds of the invention can be prepared according to Reaction Scheme I where R, R₁, and R₂ are as defined above, n is an integer from 0 to 3, m is 0 or 1, with the proviso that when m is 1, n is 0, 1, or 2, and D is —Br, —I, or —OCH₂Ph; wherein Ph is phenyl. In step (1) of Reaction Scheme I, an aminopyridine of Formula XX is treated with the condensation product generated from 2,2-dimethyl-1,3-dioxane-4,6-dione (Meldrum's acid) and triethyl orthoformate to provide an imine of Formula XXI. The reaction is conveniently carried out by adding a solution of an aminopyridine of Formula XX to a heated mixture of Meldrum's acid and triethyl orthoformate and heating the reaction at an elevated temperature. Many aminopyridines of Formula XX are commercially available; others can be prepared by known synthetic methods.

In step (2) of Reaction Scheme I, an imine of Formula XXI undergoes thermolysis and cyclization to provide a compound of Formula XXII. The reaction is conveniently carried out in a medium such as DOWTHERM A heat transfer fluid at a temperature between 230 and 250° C.

In step (3) of Reaction Scheme I, a compound of Formula XXII is nitrated under conventional nitration conditions to provide a compound of Formula XXIII. The reaction is conveniently carried out in fuming nitric acid at an elevated temperature.

In step (4) of Reaction Scheme I, a 3-nitro[1,5]naphthyridin-4-ol of Formula XXII is chlorinated using conventional chlorination chemistry to provide a 4-chloro-3-nitro[1,5]naphthyridine of Formula XXIV. The reaction is conveniently carried out by treating the compound of Formula XXII with phosphorous oxychloride in a suitable solvent such as N,N-dimethylformamide (DMF). The reaction can be carried out at ambient temperature or at an elevated temperature such as 100° C. Many compounds of Formula XXIV are known, see for example, U.S. Pat. No. 6,194,425 and the documents cited therein.

In step (5) of Reaction Scheme I, a 4-chloro-3-nitro[1,5]naphthyridine of Formula XXIV is treated with tert-butyl carbazate or an alternate carbazate to provide a carbazate compound of Formula XXV. The reaction can be carried out by adding tert-butyl carbazate to a solution of a compound of Formula XXV in a suitable solvent such as anhydrous dichloromethane in the presence of a base such as triethylamine. The reaction can be run at ambient temperature. Tertiary-butyl carbazate is commercially available (for example, from Aldrich, Milwaukee, Wis.). Many alternate carbazate reagents (for example, benzyl carbazate) may be prepared using known synthetic methods.

In step (6) of Reaction Scheme I, a carbazate compound of Formula XXV is reduced to provide a compound of Formula XXVI. The reduction can be carried out using a conventional heterogeneous hydrogenation catalyst such as platinum on carbon or palladium on carbon. For some compounds of Formula XXV, for example, compounds in which R is a halogen or when m is 1, a platinum catalyst is preferred. The reaction can be conveniently carried out on a Parr apparatus in a suitable solvent such as toluene and/or isopropanol.

Other reduction processes may be used for the reduction in step (6). For example, an aqueous solution of sodium dithionite can be added to a solution or suspension of the compound of Formula XXV in a suitable solvent such as ethanol or isopropanol. The reaction can be carried out at an elevated temperature, for example at reflux, or at ambient temperature.

In step (7) of Reaction Scheme I, a compound of Formula XXVI is (i) reacted with an acyl halide of Formula R₂C(O)Cl or R₂C(O)Br and then (ii) cyclized to provide a 1H-imidazo compound of Formula XXVII. In part (i) the acyl halide is added to a solution of a compound of Formula XXVI in a suitable solvent such as anhydrous dichloromethane in the presence of a base such as triethylamine. The reaction can be run at a reduced temperature, for example, 0° C., or at ambient temperature. In part (ii) the product of part (i) is heated in an alcoholic solvent in the presence of a base. For example, the product of part (i) is refluxed in ethanol in the presence of excess triethylamine or is heated with methanolic ammonia. Alternatively, the product of part (i) can be treated with pyridine hydrochloride in pyridine at elevated temperature.

Alternatively, step (7) can be carried out by reacting a compound of Formula XXVI with a carboxylic acid or an equivalent thereof. Suitable equivalents to a carboxylic acid include orthoesters and 1,1-dialkoxyalkyl alkanoates. The carboxylic acid or equivalent is selected such that it will provide the desired R₂ substituent in a compound of Formula XXVII. For example, triethyl orthoformate will provide a compound where R₂ is hydrogen, and triethyl orthovalerate will provide a compound where R₂ is butyl. The reaction can be run in the absence of solvent or in an inert solvent such as anhydrous toluene. The reaction is performed at an elevated temperature. Optionally a catalyst such as pyridine hydrochloride can be included.

In step (8) of Reaction Scheme I, the tert-butoxycarbonyl or alternate oxycarbonyl group is removed from a 1H-imidazo compound of Formula XXVII under acidic conditions to provide a 1H-imidazo[4,5-c][1,5]naphthyridin-1-amine of Formula XXVIII or a salt (for example, hydrochloride salt) thereof. For example, a compound of Formula XXVII is dissolved in a solution of hydrogen chloride in ethanol and heated to reflux.

In step (9) of Reaction Scheme I, a 1H-imidazo[4,5-c][1,5]naphthyridin-1-amine of Formula XXVII or a salt thereof is treated with a ketone, aldehyde, or corresponding ketal or acetal thereof, under acidic conditions to provide a compound of Formula XXIX. For example, a ketone is added to a solution of the hydrochloride salt of a compound of Formula XXVIII in a suitable solvent such as isopropanol or acetonitrile in the presence of an acid such as pyridinium p-toluene sulfonate or acetic acid, or an acid resin, for example, DOWEX W50-X1 acid resin. The ketone, aldehyde, or corresponding ketal or acetal thereof, is selected with Rᵢ and Rᵢᵢ groups that will provide the desired R₁ substituent in a 1H-imidazo[4,5-c][1,5]naphthyridin-1-amine compound of Formula XXX. For example, acetone will provide a compound where R₁ is isopropyl; benzaldehyde will provide a compound where R₁ is benzyl. The reaction is performed at an elevated temperature.

In step (10) of Reaction Scheme I, a compound of Formula XXIX is reduced to provide a 1H-imidazo[4,5-c][1,5]naphthyridin-1-amine compound of Formula XXX. The reaction can be carried out by adding sodium borohydride to a solution of a compound of Formula XXIX in a suitable solvent, for example, methanol. The reaction can be run at ambient temperature.

Alternatively, in step (9a) of Reaction Scheme I, a 1H-imidazo[4,5-c][1,5]naphthyridin-1-amine of Formula XXVIII can be treated with an aldehyde or ketone and a borohydride under acidic conditions to provide a 1H-imidazo[4,5-c][1,5]naphthyridin-1-amine of Formula XXX. For example, a 1H-imidazo[4,5-c][1,5]naphthyridin-1-amine of Formula XXVIII, dissolved in a suitable solvent such as 1,2-dichloroethane, can be treated with an aldehyde or ketone and sodium triacetoxyborohydride at room temperature.

In step (11) of Reaction Scheme I, a 1H-imidazo[4,5-c][1,5]naphthyridin-1-amine compound of Formula XXX is oxidized to provide an N-oxide of Formula XXXI using a conventional oxidizing agent that is capable of forming N-oxides. The reaction is carried out by treating a solution of a compound of Formula XXX in a suitable solvent such as chloroform or dichloromethane with 3-chloroperoxybenzoic acid at ambient temperature.

In step (12) of Reaction Scheme I, an N-oxide of Formula XXXI is aminated to provide a 1H-imidazo[4,5-c][1,5]naphthyridine-1,4-diamine of the Formula XXXII, which is a subgenus of compounds of the Formulas I, II, and IV. The reaction is carried out in two parts. In part (i) a compound of Formula XX is reacted with an acylating agent. Suitable acylating agents include alkyl- or arylsulfonyl chlorides (e.g., benzenesulfonyl chloride, methanesulfonyl chloride, and p-toluenesulfonyl chloride). In part (ii) the product of part (i) is reacted with an excess of an aminating agent. Suitable aminating agents include ammonia (e.g. in the form of ammonium hydroxide) and ammonium salts (e.g., ammonium carbonate, ammonium bicarbonate, ammonium phosphate). The reaction can be carried out by dissolving a compound of Formula XXXI in a suitable solvent such as dichloromethane, adding ammonium hydroxide to the solution, and then adding p-toluenesulfonyl chloride. The product or a pharmaceutically acceptable salt thereof can be isolated using conventional methods.

Alternatively, the oxidation of step (11) and the amination of step (12) can be carried out sequentially without isolating the product of the oxidation to provide a 1H-imidazo[4,5-c][1,5]naphthyridine-1,4-diamine of the Formula XXXII. In step (11), after the compound of Formula XXX is consumed by reaction with 3-chloroperoxybenzoic acid as described in step (11), the aminating and acylating agents are added to the reaction mixture as in step (12). The product or a pharmaceutically acceptable salt thereof can be isolated using conventional methods.

Alternatively, step (12) can be carried out by the reaction of an N-oxide of Formula XXXI with trichloroacetyl isocyanate followed by hydrolysis of the resulting intermediate to provide a 1H-imidazo[4,5-c][1,5]naphthyridine-1,4-diamine of the Formula XI. The reaction is conveniently carried out in two steps by (i) adding trichloroacetyl isocyanate to a solution of the N-oxide of Formula XXXI in a solvent such as dichloromethane and stirring at ambient temperature to provide an isolable amide intermediate. In step (ii), a solution of the intermediate in methanol is treated with a base such as sodium methoxide or ammonium hydroxide at ambient temperature. The product or a pharmaceutically acceptable salt thereof can be isolated using conventional methods.

For some embodiments, compounds of Formula XXXIII, which is a subgenus of compounds of the Formulas I, II, and IV, with the following structure:

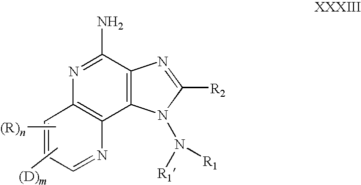

wherein R₁′ is alkyl, hydroxyalkyl, or alkoxyalkyl wherein the alkyl group contains at least 2 carbon atoms between the hydroxy or alkoxy substituent and the nitrogen atom to which R₁′ is bonded, and D, R, R₁, R₂, m, and n are as defined above, can be prepared from certain compounds shown in Reaction Scheme I. For example, a compound of Formula XXX can be treated with an alkyl aldehyde, a hydroxyalkyl aldehyde (in which the hydroxy group is appropriately protected), or an alkoxyalkyl aldehyde to generate an imine that can be reduced with a borohydride using the methods described in steps (9) and (10), or in step (9a), of Reaction Scheme I. The resulting compound can be treated according to the conditions described in steps (11) and (12) of Reaction Scheme I (followed by a protecting group removal step if necessary) to provide a compound of Formula XXXIII. Alternatively, a compound of Formula XXIV can react with a 1,1-disubstituted hydrazine of the Formula R₁—N(R₁′)—NH₂ using the conditions described in step (5) of Reaction Scheme I to provide a compound that when treated sequentially according to the conditions described in steps (6), (7), (11) and (12) of Reaction Scheme I provides a compound of Formula XXXIII. Many 1,1-disubstituted hydrazine reagents, for example, 1,1-dimethylhydrazine, are commercially available, or can be prepared using conventional methods.

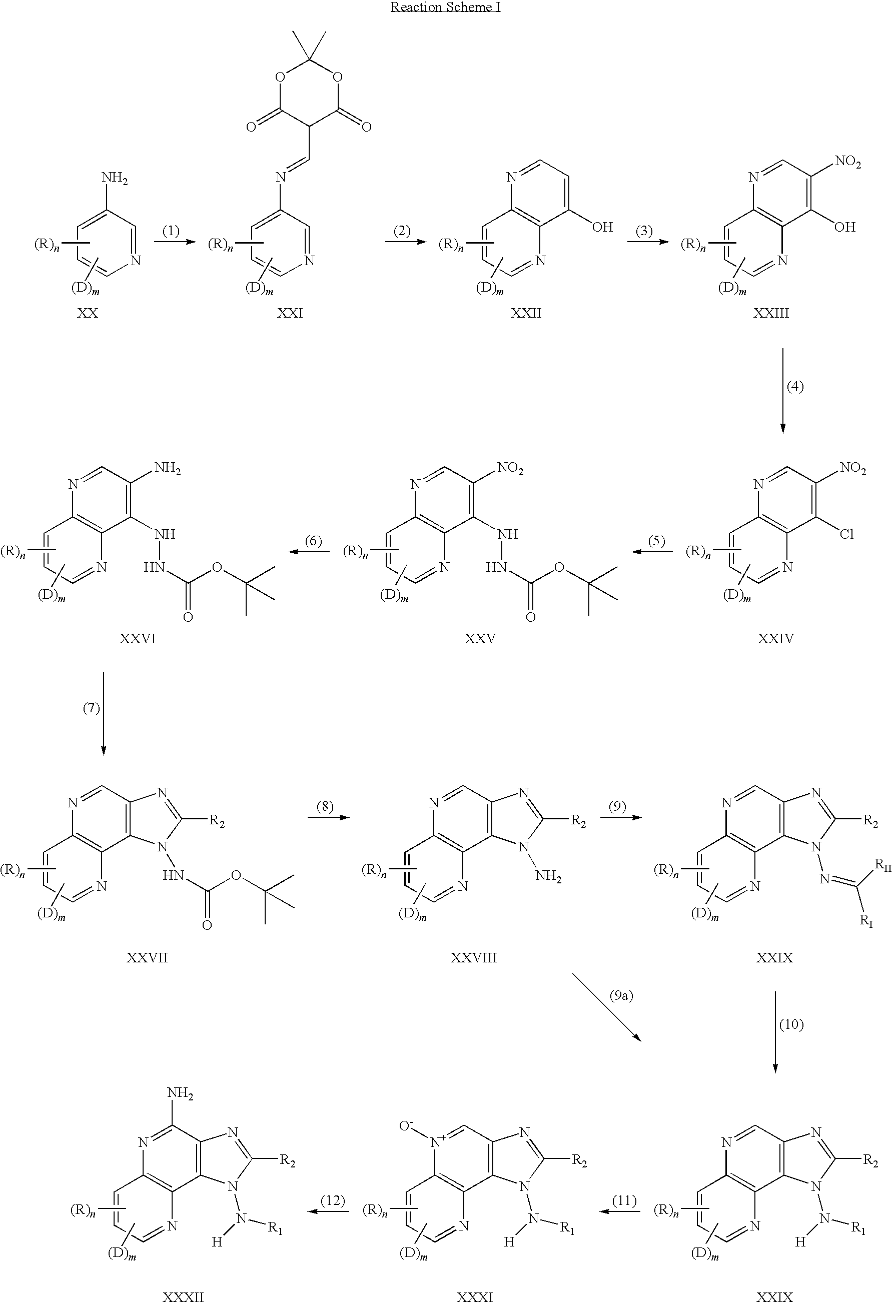

In some embodiments, further elaboration of R₁ is carried out according to Reaction Scheme II wherein D, R, R₂, R₄, m, n, X, and Y are as defined above. In step (1) of Reaction Scheme II, a 1H-imidazo[4,5-c][1,5]-naphthyridin-1-amine of Formula XXVIII or a salt thereof, prepared as described in Reaction Scheme I, undergoes a reductive alkylation with a compound that contains an acetal group and a tert-butoxycarbonyl protected amine. The reductive alkylation is carried out using the methods described in steps (9) and (10) of Reaction Scheme 1. For example, a compound of Formula XXVIII or a salt thereof can be treated with tert-butyl(3,3-diethoxypropyl)carbamate followed by a borohydride reducing agent to provide a compound of Formula X V where X is ethylene. Compounds that contain both an acetal group and a protected amino group can be prepared using conventional methods. For example, tert-butyl (3,3-diethoxypropyl)carbamate can be prepared by combining 1-amino-3,3-diethoxypropane with di-tert-butyl dicarbonate in a suitable solvent such as tetrahydrofuran (THF).

In step (2) of Reaction Scheme II, a compound of Formula XXXIV is oxidized to provide an N-oxide of Formula XXXV using the method of step (11) in Reaction Scheme I.

In step (3) of Reaction Scheme II, an N-oxide of Formula XXXV is aminated using the method of step (12) in Reaction Scheme I to provide a 1H-imidazo[4,5-c][1,5]-naphthyridin-1,4-diamine of the Formula XXXVI, which is a subgenus of compounds of the Formulas I, II, and IV.

In step (4) of Reaction Scheme II, the tert-butoxycarbonyl group is removed from a 1H-imidazo[4,5-c][1,5]-naphthyridin-1,4-diamine of the Formula XXXVI using the method of step (8) of Reaction Scheme I to provide a 1H-imidazo[4,5-c][1,5]-naphthyridin-1,4-diamine of the Formula XXXVII, which is a subgenus of compounds of the Formulas I, I, and IV.

In step (5) of Reaction Scheme II, a compound of the Formula XXXVII is converted to a compound of Formula XXXVII, which is a subgenus of compounds of the Formulas I, II, and IV, using conventional methods. For example, a 1H-imidazo[4,5-c][1,5]-naphthyridin-1,4-diamine of the Formula XXXVII or salt thereof can react with an acid chloride of Formula R₄C(O)Cl to provide a compound of Formula XXXVIII in which Y is —C(O)—. In addition, a compound of the Formula XVII can react with sulfonyl chloride of Formula R₄S(O)₂Cl or a sulfonic anhydride of Formula (R₄S(O)₂)₂O to provide a compound of Formula XXXVIII in which Y is —S(O)₂—. A compound of the Formula XXXVII can also react with a chloroformate of Formula R₄CO(O)Cl to provide a compound of Formula XXXVIII in which Y is —C(O)—O—. Numerous acid chlorides of Formula R₄C(O)Cl, sulfonyl chlorides of Formula R₄S(O)₂Cl, sulfonic anhydrides of Formula (R₄S(O)₂)₂O, and chloroformates of Formula R₄CO(O)Cl are commercially available; others can be readily prepared using known synthetic methods. The reaction can be conveniently carried out by adding the acid chloride of Formula R₄C(O)Cl, chloroformate of Formula R₄CO(O)Cl, sulfonyl chloride of Formula R₄S(O)₂Cl, or sulfonic anhydride of Formula (R₄S(O)₂)₂O to a solution of a compound of Formula XXXVII and a base such as triethylamine in a suitable solvent such as chloroform, dichloromethane, or acetonitrile. The reaction can be carried out at ambient temperature or at a sub-ambient temperature such as 0° C. The product or pharmaceutically acceptable salt thereof can be isolated using conventional methods.

Ureas of Formula XXXVIII, where Y is —C(R₇)—N(R₉)—, in which R₇ is ═O, and R₉ is as defined above, can be prepared by reacting a compound of Formula XXXVII or a salt thereof with isocyanates of Formula R₄N═C═O. Numerous isocyanates of Formula R₄N═C═O are commercially available; others can be readily prepared using known synthetic methods. The reaction can be conveniently carried out by adding the isocyanate of Formula R₄N═C═O to a cooled solution of a compound of Formula XXXVII in a suitable solvent such as dichloromethane or chloroform. Optionally, a base such as triethylamine can be added. The reaction can be carried out at ambient temperature or at a sub-ambient temperature such as 0° C. Alternatively, a compound of Formula XXXVII or a salt thereof can be treated with carbamoyl chlorides of Formula R₄N—(R₉)—C(O)Cl or Formula

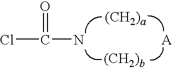

where A, a, and b are as defined above. The product or a pharmaceutically acceptable salt thereof can be isolated using conventional methods.

Thioureas of the Formula XXXVIII, where Y is —C(R₇)—N(R₉)-, in which R₇ is ═S, and R₉ is H, can be prepared by reacting a compound of Formula XXXVII or a salt thereof with thioisocyanates of Formula R₄N═C═S using the conditions described above for the reaction of a compound of Formula XXXVII with isocyanates. The product or a pharmaceutically acceptable salt thereof can be isolated using conventional methods.

Sulfamides of Formula XXXVIII, where Y is —(O)₂—N(R₆)- wherein R₆ is as defined above, can be prepared by reacting a compound of Formula XXXVII or a salt thereof with sulfuryl chloride to generate a sulfamoyl chloride in situ, and then reacting the sulfamoyl chloride with an amine of formula HN(R₆)R₄. Alternatively, sulfamides of Formula XXXVIII can be prepared by reacting a compound of Formula XXXVII with a sulfamoyl chloride of Formula R₄(R₆)N—S(O)₂Cl under the reaction conditions described above for reaction of compounds of Formula XXXVII with sulfonyl chlorides. The product or a pharmaceutically acceptable salt thereof can be isolated using conventional methods. Many amines of Formula HN(R₆)R₄, and some sulfamoyl chlorides of Formula R₄(R)N—S(O)₂Cl are commercially available; others can be prepared using known synthetic methods.

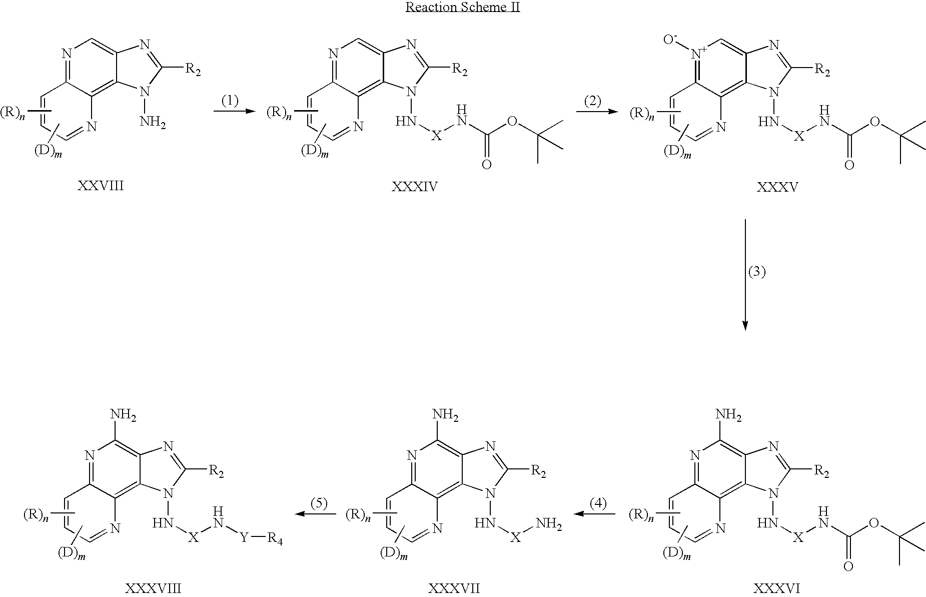

Compounds of the invention can be prepared according to Reaction Scheme III wherein R, R₁, R₂, Rᵢ, Rᵢᵢ, D, m, and n are as defined above. In step (1) of Reaction Scheme III, aminomalonitrile of Formula XXXIX, which is available commercially as the p-toluenesulfonic acid salt, is reacted with an orthoester to generate an imidate intermediate, which is treated with tert-butyl carbazate to generate a compound of Formula XL. As in step (7) of Reaction Scheme I, the orthoester is selected such that it will provide the desired R₂ substituent in a compound of Formula XL. The reaction is conveniently carried out by heating a solution of aminomalonitrile p-toluenesulfonate and the orthoester in a suitable solvent such as tetrahydrofuran (THF) in the presence of triethylamine. The solution is allowed to cool to ambient temperature and the tert-butyl carbazate is added.

In step (2) of Reaction Scheme III, a solution of a compound of Formula XL in diiodomethane or bromoform is treated with isoamyl nitrite or tert-butyl nitrite at an elevated temperature to yield a compound of Formula XLI, where Hal is defined as —Br or —I.

In step (3) of Reaction Scheme III, an iodo or bromo-substituted compound of Formula XLI undergoes a transition-metal catalyzed cross coupling reaction with a reagent of Formula XLII to form a compound of Formula XLIII. Reagents of Formula XLII, where M is, for example, —B(OH)₂, —B(O-alkyl)₂, —Sn(alkyl)₃, and —Zn-Halide, are known to undergo coupling reactions. One reagent of Formula XLII is commercially available (2,2-dimethyl-N-[3-(4,4,5,5-tetramethyl-1,3,2-dioxaborolan-2-yl)pyridin-4-yl]propanamide, CB Research and Development, Inc. in New Castle, Del.); others can be prepared using known synthetic methods. For example, tert-butylcarbonyl protected aminopyridines undergo directed ortho metalation in the presence of butyllithium reagents. The resulting organolithium intermediate reacts with electrophiles such as B(O-alkyl)₃ and ClSn(alkyl)₃ to provide compounds of Formula XLII, where M is —B(O-alkyl)₂ or —B(OH)₂ and —Sn(alkyl)₃, respectively.

In step (3), a Suzuki coupling reaction is conveniently carried out by heating a mixture of the compound of Formula XLI, palladium (II) acetate, triphenylphosphine, and a boron reagent of Formula XLII, where M is —B(OH)₂ or —B(O-alkyl)₂, in the presence of a base such as sodium carbonate. The reaction is carried out in a suitable solvent or solvent mixture such as n-propanol:water and can be heated at an elevated temperature such as 100° C.

In step (4) of Reaction Scheme III, the tert-butoxycarbonyl is removed from a compound of Formula XLIII using acidic conditions, for example, the method described in step (8) of Reaction Scheme I to yield a compound of Formula XLIV or a salt thereof.

In step (5) of Reaction Scheme III, a compound of Formula XLIV or a salt thereof is treated with a ketone, aldehyde, or corresponding ketal or acetal thereof according to the conditions described in step (9) of Reaction Scheme I to provide a compound of Formula XLV.

In step (6) of Reaction Scheme III, a compound of Formula XLV is reduced to provide a compound of Formula XLVI using the method described in step (10) of Reaction Scheme I. Alternatively, steps (5) and (6) can be performed with one pot using the procedure described in step (9a) of Reaction Scheme I.

In step (7) of Reaction Scheme III, a compound of Formula XLVI undergoes a base-promoted intramolecular cyclization followed by hydrolysis of the tert-butylcarbonyl group to provide a compound of Formula XLVII, which is a subgenus of Formulas III, and V. The reaction is conveniently carried out by heating a compound of Formula XLVI with potassium tert-butoxide in a suitable solvent such as ethanol at an elevated temperature such as the reflux temperature of the solvent. The product or pharmaceutically acceptable salt thereof can be isolated using conventional methods.

For some embodiments, compounds of Formula XLVIII, with the following structure:

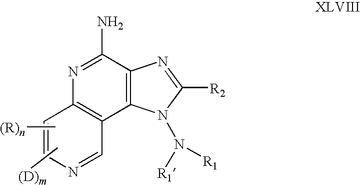

wherein R₁′ is alkyl, hydroxyalkyl, or alkoxyalkyl, wherein the alkyl group contains at least 2 carbon atoms between the hydroxy or alkoxy substituent and the nitrogen atom to which R₁′ is bonded, and D, R, R₁, R₂, m, and n are as defined above, can be prepared from compound of Formula XLVI. A compound of Formula XLVI can be treated with an alkyl aldehyde, a hydroxyalkyl aldehyde (in which the hydroxy group is appropriately protected), or an alkoxyalkyl aldehyde as described above for the synthesis of compounds of Formula XXXIII. The resulting compound can be treated according to the conditions described in step (7) of Reaction Scheme III to provide a compound of Formula XLVIII.

In some embodiments, R₁ can be further elaborated using conventional synthetic methods. For example, a compound of Formula XLVII in which R₁ is -X—NH-Boc where X is as defined above can be deprotected using the method of step (8) of Reaction Scheme I to yield a primary amine that can be functionalized using the reagents and methods described in step (5) of Reaction Scheme II to yield compounds of Formula XLVII where R₁ is equal to -X—N(R₆)—Y—R₄ wherein X, Y, and R₄ are as described above and R₆ is H.

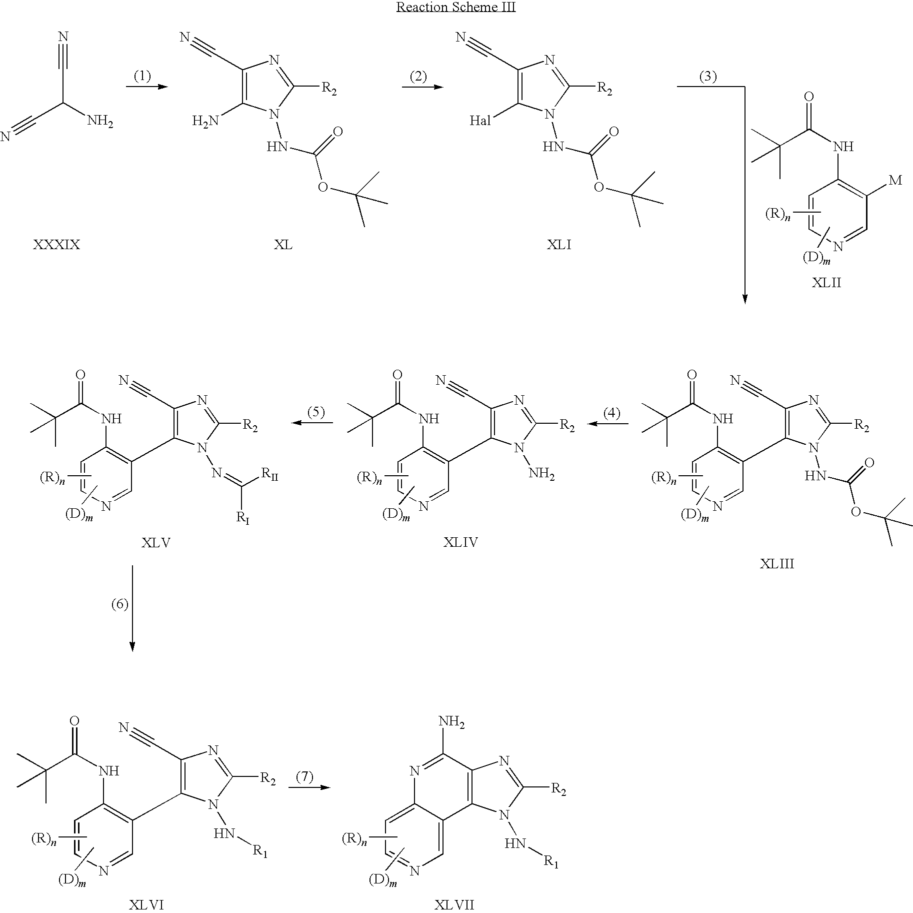

1H-Imidazo[4,5-c][1,7]naphthyridine-1,4-diamines and 1H-imidazo[4,5-c][1,8]naphthyridine-1,4-diamines of the Formula LXIII where R, R₁, R₂, m, and n are as defined above, D is —Br, —I, or —OCH₂Ph wherein Ph is phenyl, and A contains the necessary atoms to provide XLIX as the following two compounds:

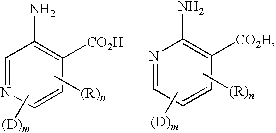

can be prepared according to Reaction Scheme IV.

In step (1) of Reaction Scheme IV, a 3-aminoisonicotinic acid or 2-aminonicotinic acid of Formula XLIX is reacted with acetic anhydride by heating to provide a 2-methyl-4H-pyrido[3,4-d][1,3]oxazin-4-one or 2-methyl-4H-pyrido[2,3-d][1,3]oxazin-4-one of Formula L.

In step (2) of Reaction Scheme IV, a 2-methyl-4H-pyrido[3,4-d][1,3]oxazin-4-one or 2-methyl-4H-pyrido[2,3-d][1,3]oxazin-4-one of Formula L is reacted with sodium azide in a suitable solvent such as acetic acid to provide a tetrazolyl isonicotinic acid or tetrazolyl nicotinic acid of Formula LI.

In step (3) of Reaction Scheme IV, an acid of Formula LI is esterified to provide a compound of Formula LII, where Rᵢᵢᵢ is an allyl group such as methyl or ethyl. The esterification may be carried out using conventional methods. For example, the acid may be esterified in acetone with potassium carbonate and ethyl iodide or by reacting with dimethylformamide diethyl acetal in a suitable solvent such as dichloromethane.

In step (4) of Reaction Scheme IV, a compound of Formula LII is cyclized to provide a tetrazolo[1,5-a][1,7]naphthyridin-5-ol or tetrazolo[1,5-a][1,8]naphthyridin-5-ol of Formula LIII. The reaction may be carried out by reacting the compound of Formula LII with an alkoxide in a suitable solvent, e.g., potassium ethoxide in DMF at ambient temperature.

In step (5) of Reaction Scheme IV, a compound of Formula LIII is nitrated under conventional nitration conditions to provide a compound of Formula LIV. The reaction is conveniently carried out by adding nitric acid to the compound of Formula LIII in a suitable solvent such as propionic acid and heating the mixture at an elevated temperature.

In step (6) of Reaction Scheme IV, a compound of Formula LIV is converted to a triflate of Formula LV. A compound of Formula LIV is reacted with trifluoromethanesulfonic anhydride in the presence of a base such as triethylamine. The reaction is carried out in a suitable solvent such as dichloromethane at 0° C. Some compounds of the Formula LV have been synthesized previously; see for example, U.S. Pat. No. 6,194,425.

In step (7) of Reaction Scheme IV, a compound of Formula LV is treated with tert-butyl carbazate or an alternate carbazate to provide a carbazate compound of Formula LVI. The reaction can be carried out using the conditions described in step (5) of Reaction Scheme I.

Alternatively, a compound of Formula LIV can be chlorinated with a suitable chlorinating agent such phosphorus oxychloride to provide a 5-chloro-4-nitrotetraazolo[1,5-a][1,7]naphthyridine or 5-chloro-4-nitrotetraazolo[1,5-a][1,8]naphthyridine that can be treated with a tert-butyl carbazate according to the conditions described in step (5) of Reaction Scheme I to provide a compound of Formula LVI. The chlorination reaction can be carried out in an inert solvent or if appropriate in neat phosphorus oxychloride. The reaction can be carried out at an elevated temperature such as 90° C.

Steps (8), (9), (10), (11), and (12) of Reaction Scheme IV can carried out using the conditions described in steps (6), (7), (8), (9), and (10), respectively, of Reaction Scheme I to convert a compound of Formula LVI into a compound of Formula LXI.

In step (13) of Reaction Scheme IV, the tetrazolo ring can be removed from a 6H-imidazo[4,5-c]tetraazolo[1,5-a][1,7]naphthyridin-6-amine or 9H-imidazo[4,5-c]tetraazolo[1,5-a][1,8]naphthyridin-9-amine of Formula LXI by reaction with triphenylphosphine to form an N-triphenylphosphinyl intermediate of Formula LXII. The reaction with triphenylphosphine can be run in a suitable solvent such as toluene or 1,2-dichlorobenzene under an atmosphere of nitrogen with heating, for example at the reflux temperature.

In step (14) of Reaction Scheme IV, an N-triphenylphosphinyl intermediate of Formula LXII is hydrolyzed to provide a 1H-imidazo[4,5-c][1,7]naphthyridine-1,4-diamine or 1H-imidazo[4,5-c][1,8]naphthyridine-1,4-diamine of Formula LXIII, which represents a subgenus of Formulas I and II. The hydrolysis can be carried out by general methods well known to those skilled in the art, for example, by heating in a lower alkanol in the presence of an acid such as trifluoroacetic acid or hydrochloric acid. The product can be isolated from the reaction mixture using conventional methods as the compound of Formula LXIII or as a pharmaceutically acceptable salt thereof.

For some embodiments, compounds of Formula LXIV, with the following structure:

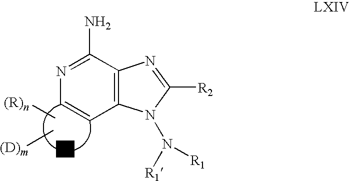

wherein R₁′ is alkyl, hydroxyalkyl, or alkoxyalkyl, wherein the alkyl group contains at least 2 carbon atoms between the hydroxy or alkoxy substituent and the nitrogen atom to which R₁′ is bonded, and D, R, R₁, R₂, m, and n are as defined above, can be prepared from compound of Formula LXI. A compound of Formula LXI can be treated with an alkyl aldehyde, a hydroxyalkyl aldehyde (in which the hydroxy group is appropriately protected), or an alkoxyalkyl aldehyde followed by a reducing agent as described above for the synthesis of compounds of Formula XXXIII. The resulting compound can be treated according to the conditions described in steps (13) and (14) of Reaction Scheme IV to provide a compound of Formula LXIV.

In some embodiments, R₁ can be further elaborated using conventional synthetic methods. For example, a compound of Formula LXI in which R₁ is -X—NH-Boc, where X is as defined above, can be converted into a compound of Formula LXIII in which R₁ is -X—NH₂ or a salt thereof during steps (13) and (14) of Reaction Scheme IV. A compound of Formula LXIII in which R₁ is -X—NH₂ or a salt thereof can be functionalized using the reagents and methods described in step (5) of Reaction Scheme II to yield compounds of Formula LXIII where R₁ is equal to -X—N(R₆)—Y—R₄ wherein X, Y, and R₄ are as described above and R₆ is H.

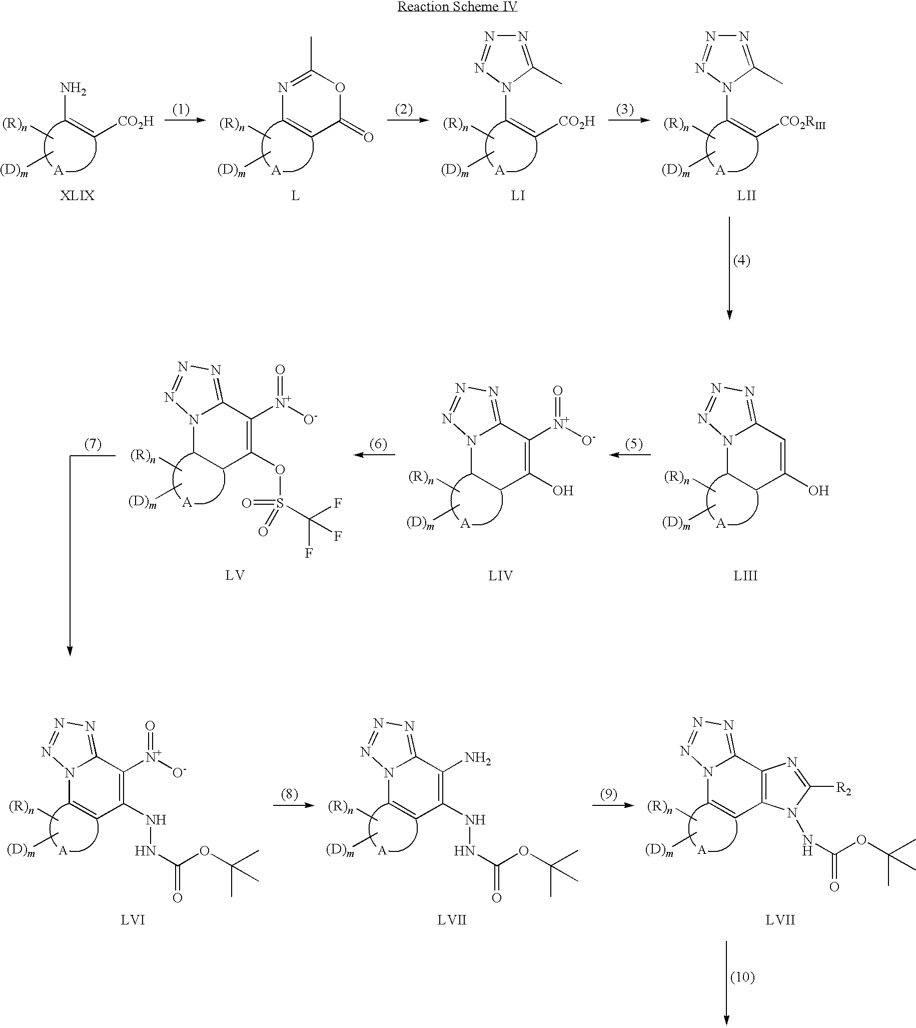

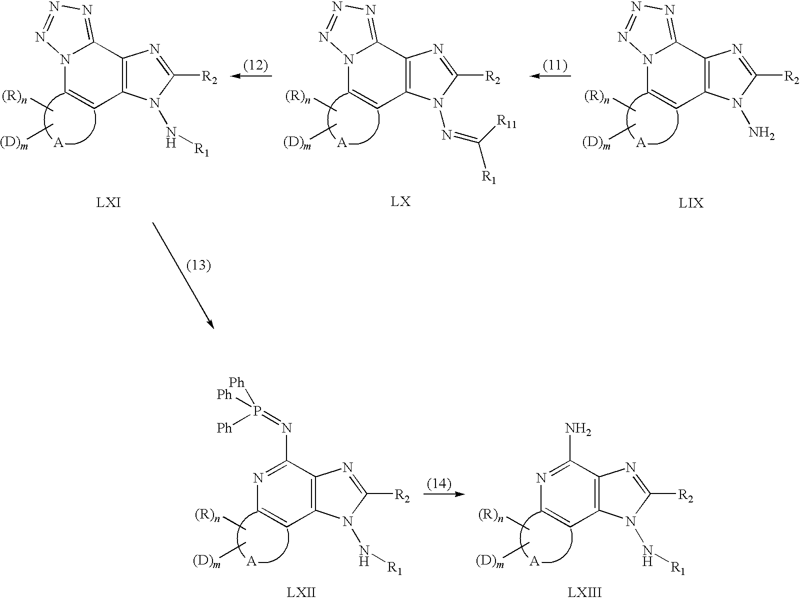

Compounds of the invention can be prepared according to Reaction Scheme V where R_{A1}, R_{B1}, R₁, R₂, Rᵢ, and Rᵢᵢ are as defined above and P is a benzyl or 4-methoxybenzyl protecting group. In step (1) of Reaction Scheme V, a 2,4-dichloro-3-nitropyridine of Formula LXV is treated with tert-butyl carbazate or an alternate carbazate to provide a carbazate compound of Formula LXVI. The reaction can be carried out by adding tert-butyl carbazate to a solution of a compound of Formula LXV in a suitable solvent such as anhydrous DMF in the presence of a base such as triethylamine. The reaction can be run at elevated temperature, for example, 70° C. Many 2,4-dichloro-3-nitropyridines of Formula LXV are known and can be prepared using known synthetic methods (see, for example, Dellaria, et al., U.S. Pat. No. 6,525,064 and references cited therein). Tertiary-butyl carbazate is commercially available (for example, from Aldrich, Milwaukee, Wis.). Many alternate carbazate reagents (for example, benzyl carbazate) may be prepared using known synthetic methods.

In step (2) of Reaction Scheme V, a compound of Formula LXVI is treated with either dibenzylamine or N,N-bis(4-methoxybenzyl)amine to yield a compound of Formula LXVII. The reaction is conveniently carried by combining a compound of Formula LXVI with triethylamine and excess dibenzylamine or N,N-bis(4-methoxybenzyl)amine. The reaction can be run at elevated temperature.

In step (3) of Reaction Scheme V, the nitro group in a compound of Formula LXVII is reduced to yield a compound of Formula LXVIII. The reaction is carried out as described for step (6) of Reaction Scheme I.

In step (4) of Reaction Scheme V, a compound of Formula LXVIII is converted into a 1H-imidazo[4,5-c]pyridine-1,4-diamine of Formula LXIX. The reaction is carried out in two steps using the methods described in step (7) of Reaction Scheme I.

In step (5) of Reaction Scheme V, a compound of Formula LXIX is treated with ethanolic hydrogen chloride as described in step (8) of Reaction Scheme I to afford a compound of Formula LXX.

In step (6) of Reaction Scheme V, a compound of Formula LXX is treated with a ketone, aldehyde, or corresponding ketal or acetal thereof, under acidic conditions described in step (9) of Reaction Scheme I to provide a compound of Formula LXXI.

In step (7) of Reaction Scheme V, a compound of Formula LXXI is reduced according to the conditions described in step (10) of Reaction Scheme I to provide a compound of Formula LXXII.

In step (8) of Reaction Scheme V, a compound of Formula LXXII is deprotected to yield a compound of Formula LXXIII. A compound of Formula LXXII, where P is equal to a benzyl group, can be deprotected using ammonium formate and a heterogeneous catalyst such as palladium on carbon in a solvent mixture comprised of ethanol and methanol. The reaction is carried out at the reflux temperature of the solvent or solvent system. A compound of Formula LXXII, where P is equal to a 4-methoxybenzyl group, can be deprotected with trifluoroacetic acid. The reaction is carried out at ambient temperature. The product or a pharmaceutically acceptable salt thereof can be isolated by conventional methods.

For some embodiments, compounds of Formula LXXIV, with the following structure:

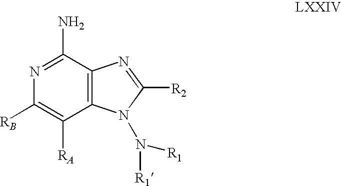

wherein R₁′ is alkyl, hydroxyalkyl, or alkoxyalkyl, wherein the alkyl group contains at least 2 carbon atoms between the hydroxy or alkoxy substituent and the nitrogen atom to which R₁′ is bonded, and R_{A1}, R_{B1}, R₁, and R₂ are as defined above, can be prepared from compound of Formula LXXII. A compound of Formula LXXII can be treated with an alkyl aldehyde, a hydroxyalkyl aldehyde (in which the hydroxy group is appropriately protected), or an alkoxyalkyl aldehyde followed by a reducing agent as described above for the synthesis of compounds of Formula XXXIII. The resulting compound can be treated according to the conditions described in step (8) of Reaction Scheme V to provide a compound of Formula LXXIV.

In some embodiments, R₁ can be further elaborated using conventional synthetic methods. For example, a compound of Formula LXXII in which R₁ is -X—NH-Boc where X is as defined above and P is a 4-methoxybenzyl group can be converted into a compound of Formula LXXI in which R₁ is -X—NH₂ or a salt thereof during step (8) of Reaction Scheme V. A compound of Formula LXXIII in which R₁ is -X—NH₂ or a salt thereof can be functionalized using the reagents and methods described in step (5) of Reaction Scheme II to yield compounds of Formula LXXIII where R₁ is equal to -X—N(R₆)—Y—R₄ wherein X, Y, and R₄ are as described above and R₆ is H.

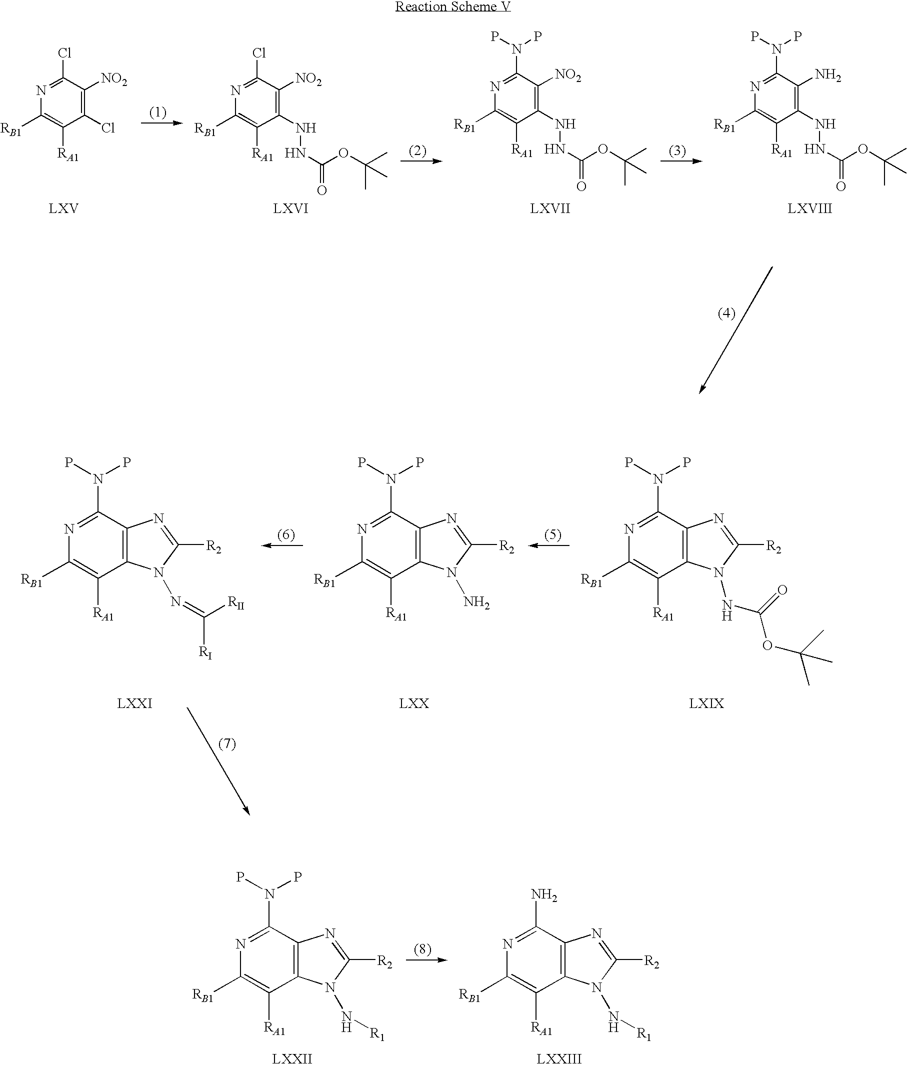

Compounds of the invention can be prepared according to Reaction Scheme VI where B contains the necessary atoms to provide LXXV as the following four compounds:

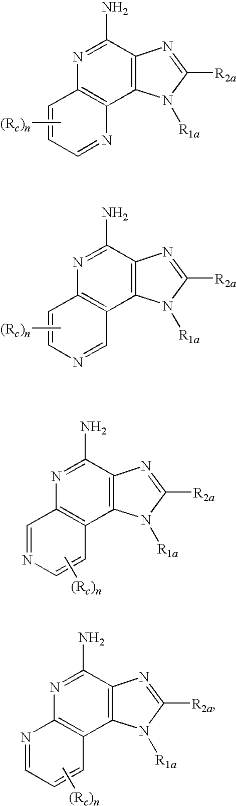

and C contains the necessary atoms to provide LXXVI as the following four compounds:

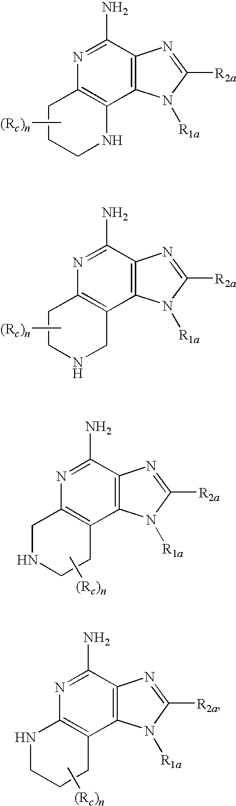

n and R₁′ are as defined above; each R_{c}, R₁ₐ, and R₂ₐ are a subset of R, R₁, and R₂, respectively, as defined above, which do not include those groups that one skilled in the art would recognize as being susceptible to reduction under the acidic hydrogenation conditions in step (1). These susceptible groups include, for example, alkenyl, alkynyl, and aryl groups, and groups bearing nitro substituents.

In step (1) of Reaction Scheme VI, a compound of Formula LXXV is reduced to provide a compound of Formula LXXVI, which is a subgenus of compounds of the Formulas I and II. The reaction can be conveniently carried out by suspending or dissolving a compound of Formula LXXV in trifluoroacetic acid, adding platinum(IV) oxide, and hydrogenating. The reaction can be carried out in a Parr apparatus. The product or a pharmaceutically acceptable salt thereof can be isolated using conventional methods.

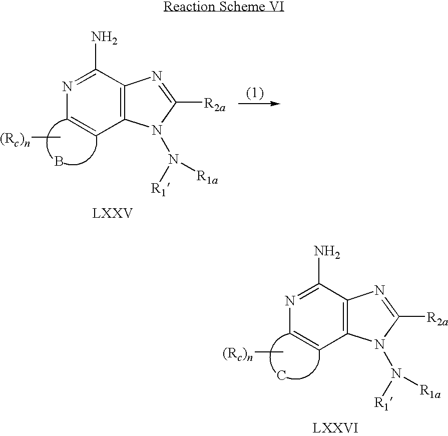

Compounds of the invention can also be prepared according to Reaction Scheme VII, wherein R, R₁, R₁′, R₂, and B are as defined above; n is 0, 1, or 2; and R₃ₐ is —O—R₄ₐ′—O—X′—R₄′, —O—X′—Y—R₄′, or —O—X′—R₅′; where R₄′, R₅′, X′ and Y′ are as defined above, and R₄ₐ′ is aryl or heteroaryl where the aryl or heteroaryl groups can be unsubstituted or substituted as defined in R₄′ above. Compounds of Formula LXXVII can be prepared according to the methods described in Reaction Schemes I, II, m, and IV, wherein D is —OCH₂Ph. In step (1) of Reaction Scheme VII, the benzyl group in a benzyloxy-substituted compound Formula LXXVII is cleaved to provide a compound of Formula LXXVIII. The cleavage is conveniently carried out on a Parr apparatus under hydrogenolysis conditions using a suitable heterogeneous catalyst such as palladium or platinum on carbon in a solvent such as ethanol. Alternatively, the reaction can be carried out by transfer hydrogenation in the presence of a suitable hydrogenation catalyst. The transfer hydrogenation is carried out by adding ammonium formate to a solution of a compound of Formula LXXVII in a suitable solvent such as ethanol in the presence of a catalyst such as palladium on carbon. The reaction is carried out at an elevated temperature, for example, the refluxing temperature of the solvent. The product or pharmaceutically acceptable salt thereof can be isolated using conventional methods.

In step (2) of Reaction Scheme VII, a hydroxy-substituted compound of Formula LXXVIII is converted to an ether-substituted compound of Formula LXXIX using a Williamson-type ether synthesis. The reaction is effected by treating a compound of Formula LXXVIII with an aryl or alkyl halide of Formula Halide-R₄ₐ′, Halide-alkylene-R₄′, Halide-alkylene-Y′—R₄′ or Halide-alkylene-R₅′ in the presence of a base. Numerous reagents of Formulas Halide-R₄′, Halide-alkylene-R₄′, Halide-alkylene-Y′—R₄′ or Halide-alkylene-R₅′ are commercially available, including substituted benzyl bromides and chlorides, substituted or unsubstituted alkyl or arylalkylenyl bromides and chlorides, and substituted fluorobenzenes. Other reagents of these Formulas can be prepared using conventional synthetic methods. The reaction is conveniently carried out by combining a reagent of Formula Halide-R₄ₐ′, Halide-alkylene-R₄′, Halide-alkylene-Y′—R₄′ or Halide-alkylene-R₅′ with a hydroxy-substituted compound of Formula LXXVIII in a solvent such as DMF in the presence of a suitable base such as cesium carbonate. Optionally, catalytic tetrabutylammonium bromide can be added. The reaction can be carried out at ambient temperature or at an elevated temperature, for example 65° C. or 85° C., depending on the reactivity of the aryl or alkyl halide. The product or pharmaceutically acceptable salt thereof can be isolated using conventional methods.

Alternatively, step (3) may be carried out using the Ullmann ether synthesis, in which an alkali metal aryloxide of a compound of Formula LXXVIII reacts with an aryl halide in the presence of copper salts, to provide a compound of Formula LXXIX, where R₃ₐ is —O—R₄ₐ′ or —O—X′-R₄′ wherein X′ is an arylene or heteroarylene.

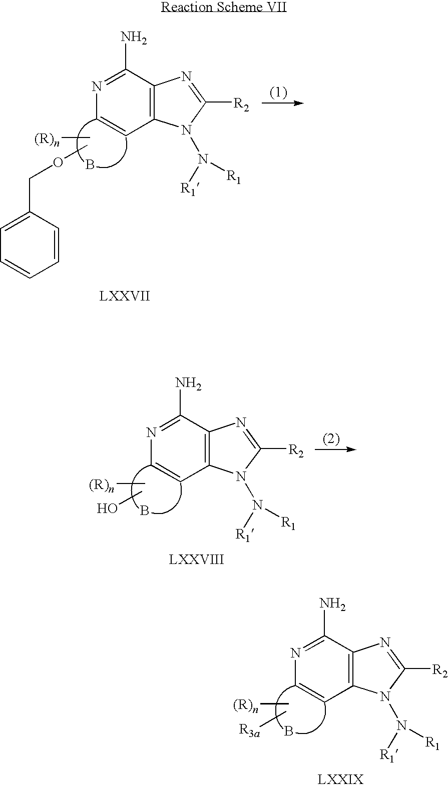

Compounds of the invention can also be prepared according to Reaction Scheme VIII, wherein R, R₁, R₁′, R₂, and B are as defined above; n is 0, 1, or 2; Hal is —Br or —I; and R_{3b} is -R₄ₐ′, -X′ₐ-R₄′, -X′_{b}—Y′—R₄′, or -X′_{b}-R₅′, where R₄ₐ′ is aryl or heteroaryl where the aryl or heteroaryl groups can be unsubstituted or substituted as defined in R₄′ above; X′ₐ is alkenylene; X′_{b} is arylene, heteroarylene, and alkenylene interrupted or terminated by arylene or heteroarylene; and R₄′, R₅′, and Y′ are as defined above. Compounds of Formulas LXXX can made according to the methods described in Reaction Schemes I, II, III, and IV, wherein D is —Br or —I. Step (1) of Reaction Scheme VIII can be carried out using known palladium-catalyzed coupling reactions such as the Suzuki coupling and the Heck reaction. For example, a halogen substituted compound of Formula LXXX undergoes Suzuki coupling with a boronic acid of Formula R_{3b}—B(OH)₂, an anhydride thereof, or a boronic acid ester of Formula R_{3b}—B(O-alkyl)₂ to provide a compound of Formula LXXXI. The coupling is carried out by combining a compound of Formula LXXX with a boronic acid or an ester or anhydride thereof in the presence of palladium (II) acetate, triphenylphosphine, and a base such as sodium carbonate in a suitable solvent such as n-propanol. The reaction can be carried out at an elevated temperature, for example, at the reflux temperature. Numerous boronic acids of Formula R_{3b}—B(OH)₂, anhydrides thereof, and boronic acid esters of Formula R_{3b}—B(O-alkyl)₂ are commercially available; others can be readily prepared using known synthetic methods. See, for example, Li, W. et al, J. Org. Chem., 67, 5394-5397 (2002). The product of Formula LXXXI or a pharmaceutically acceptable salt thereof can be isolated by conventional methods.

The Heck reaction can also be used in step (1) of Reaction Scheme VII to provide compounds of Formula LXXX, wherein R_{3b} is -X′ₐ-R₄ₐ′ and X′ₐ—Y′—R₄′. The Heck reaction is carried out by coupling a compound of Formula LXXX with a compound of the Formula H₂C═C(H)—R₄ₐ′ or H₂C═C(H)—Y′—R₄′. Several of these vinyl-substituted compounds are commercially available; others can be prepared by known methods. The reaction is conveniently carried out by combining the compound of Formula LXXX and the vinyl-substituted compound in the presence of palladium (II) acetate, triphenylphosphine or tri-ortho-tolylphosphine, and a base such as triethylamine in a suitable solvent such as acetonitrile or toluene. The reaction can be carried out at an elevated temperature such as 100-120° C. under an inert atmosphere. The product of Formula LXXXI or pharmaceutically acceptable salt thereof can be isolated using conventional methods.

Compounds of Formula LXXXI, wherein R_{3b} is -X′_{c}-R₄′, X′_{c} is alkynylene, and R₄′ is as defined above, can also be prepared by palladium catalyzed coupling reactions such as the Stille coupling or Sonogashira coupling. These reactions are carried out by coupling a compound of Formula LXXX with a compound of the Formula (alkyl)₃Sn—C≡C—R₄′, (alkyl)₃Si—C≡C—R₄′, or H—C≡C—R₄′.

Compounds of the invention, wherein R_{3c} is -X′_{d}-R₄′, -X′_{d}—Y′—R₄′, -X′ₑ—Y′—R₄′, or -X′ₑ-R₅′, where X′_{d} is alkylene; X′ₑ is alkylene interrupted or terminated by arylene or heteroarylene; and R₄′, R₅′, and Y′ are as defined above, can be prepared as shown in step (2) of Reaction Scheme VIII. In step (2) of Reaction Scheme VIII, a compound of Formula LXXXI, wherein R_{3b} is -X′ₐ-R₄′, -X′ₐ—Y′—R₄′, -X′_{b}—Y′—R₄′, -X′_{b}-R₅′, or -X_{c}′-R₄′, where X′_{b} is alkenylene interrupted or terminated by arylene or heteroarylene, and X′ₐ, X′_{c}, Y′, R₄′, and R₅′ are as defined above, is reduced to provide a compound of Formula LXXXII. The reduction can be carried out by hydrogenation using a conventional heterogeneous hydrogenation catalyst such as palladium on carbon. The reaction can conveniently be carried out on a Parr apparatus in a suitable solvent such as ethanol, methanol, or mixtures thereof. The product or pharmaceutically acceptable salt thereof can be isolated using conventional methods.

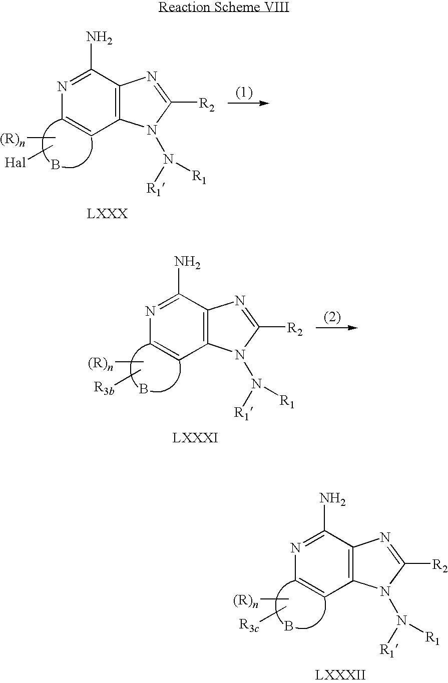

Compounds of the invention can be prepared according to Reaction Scheme IX where R_{A1}, R_{B1}, R₁, R₂, Rᵢ, and Rᵢᵢ are as defined above and P is a benzyl or 4-methoxybenzyl protecting group.

In step (1) of Reaction Scheme IX, the nitro group in a compound of Formula LXVI is reduced to yield a compound of Formula LXXXIII. The reaction is carried out as described for step (6) of Reaction Scheme I.

In step (2) of Reaction Scheme IX, a compound of Formula LXXXIII is converted into a 1H-imidazo[4,5-c]pyridin-1amine of Formula LXXXIV. The reaction is carried out in two steps using the methods described in step (7) of Reaction Scheme I.

In step (3) of Reaction Scheme IX, a compound of Formula LXXXIV is treated with ethanolic hydrogen chloride as described in step (8) of Reaction Scheme I to afford a compound of Formula LXXXVI.

In step (4) of Reaction Scheme IX, a compound of Formula LXXXV is treated with a ketone, aldehyde, or corresponding ketal or acetal thereof, under acidic conditions described in step (9) of Reaction Scheme I to provide a compound of Formula LXXXVI.

In step (5) of Reaction Scheme IX, a compound of Formula LXXXVI is reduced according to the conditions described in step (10) of Reaction Scheme I to provide a compound of Formula LXXXVII.

In step (6) of Reaction Scheme IX, a compound of Formula LXXXVII is treated with either benzylamine or N-(4-methoxybenzyl)amine to yield a compound of Formula LXXXVIII. The reaction is conveniently carried by combining a compound of Formula LXXXVII with excess benzylamine or N-(4-methoxybenzyl)amine and excess pyridine hydrochloride in a suitable solvent such as methanol in a pressure vessel. The reaction can be run at an elevated temperature, such as 150° C. Alternatively, a compound of Formula LXXXVII is combined with excess N-(4-methoxybenzyl)amine and excess pyridine hydrochloride in a suitable solvent such as 2,2,2-trifluoroethanol and heated (150-160° C.) in a microwave reactor.

In step (7) of Reaction Scheme IX, a compound of Formula LXXXVIII is deprotected to yield a compound of Formula LXXIII. A compound of Formula LXXXVIII, where P is equal to a benzyl group, can be deprotected using ammonium formate and a heterogeneous catalyst such as palladium on carbon in a solvent mixture comprised of ethanol and methanol. The reaction is carried out at the reflux temperature of the solvent or solvent system. A compound of Formula LXXXVIII, where P is equal to a 4-methoxybenzyl group, can be deprotected with trifluoroacetic acid. The reaction is carried out at ambient temperature. The product or a pharmaceutically acceptable salt thereof can be isolated by conventional methods.

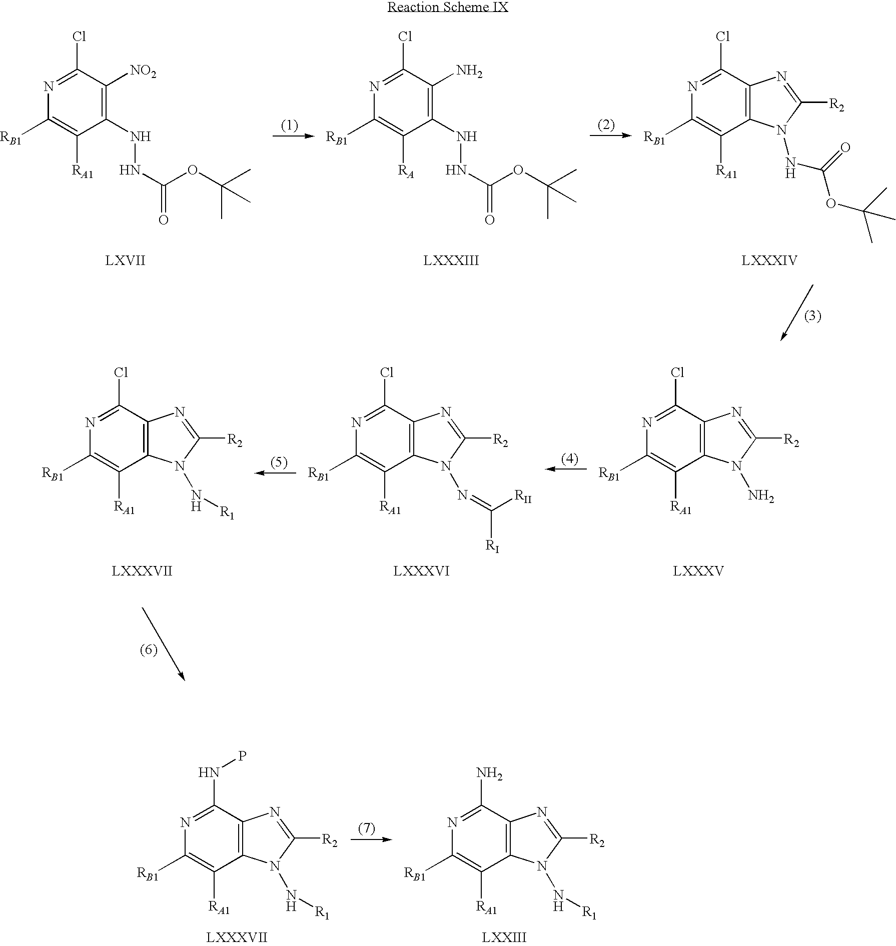

Compounds of the invention can also be prepared using the synthetic routes described in the EXAMPLES below.

Prodrugs can be prepared in a variety of ways. For example, a compound wherein R₂ is -alkylenyl-OH can be converted into a prodrug wherein R₂ is, for example, -alkylenyl-O—C(R₇)-R₄, -alkylenyl-O—C(R₇)—O—R₄, or -alkylenyl-O—C(R₇)—N(R₆)-R₄, wherein R₄, R₆, and R₇ are as defined above, using methods known to one skilled in the art. In addition, a compound wherein R is hydroxy may also be converted to an ester, an ether, a carbonate, or a carbamate. For any of these compounds containing an alcohol functional group, a prodrug can be formed by the replacement of the hydrogen atom of the alcohol group with a group such as C₁₋₆ alkanoyloxymethyl, 1-(C₁₋₆ alkanoyloxy)ethyl, 1-methyl-1-(C₁₋₆ alkanoyloxy)ethyl, C₁₋₆ alkoxycarbonyloxymethyl, N—(C₁₋₆ alkoxycarbonyl)aminomethyl, succinoyl, C₁₋₆alkanoyl, α-aminoC₁₋₄ alkanoyl, arylacyl, —P(O)(OH)₂, —P(O)(O—C₁₋₆ alkyl)₂, C₁₋₆ alkoxycarbonyl, C₁₋₆ alkylcarbamoyl, and α-aminoacyl or α-aminoacyl-α-aminoacyl, where each α-aminoacyl group is independently selected from the naturally occurring racemic, D- or, L-amino acids. For compounds containing an alcohol functional group, particularly useful prodrugs are esters made from carboxylic acids containing one to six carbon atoms, unsubstituted or substituted benzoic acid esters, or esters made from naturally occurring racemic, D-, or L-amino acids.

Prodrugs can also be made from a compound containing an amino group by conversion of the amino group to a functional group such as an amide, carbamate, urea, amidine, or another hydroylizable group using conventional methods. A prodrug of this type can be made by the replacement of a hydrogen atom in an amino group, particularly the amino group at the 4-position, with a group such as —C(O)—R′, α-aminoacyl, α-aminoacyl-α-aminoacyl, —C(O)—O—R′, —C(O)—N(R″″)-R′, —C(═NY₂)—R′, —CH(OH)—C(O)—OY₂, —CH(OC₁₋₄ alkyl)Y₀, —CH₂Y₁, or —CH(CH₃)Y₁; wherein R′ and R″″ are each independently C₁₋₁₀ alkyl, C₃₋₇ cycloalkyl, phenyl, or benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂; each α-aminoacyl group is independently selected from the naturally occurring racemic, D-, or L-amino acids; Y₂ is hydrogen, C₁₋₆ alkyl, or benzyl; Y₀ is C₁₋₆ alkyl, carboxyC₁₋₆ alkyl, aminoC₁₋₄ alkyl, mono-N—C₁₋₆ alkylaminoC₁₋₄ alkyl, or di-N,N—C₁₋₆ alkylaminoC₁₋₄ alkyl; and Y₁ is mono-N—C₁₋₆ alkylamino, di-N,N—C₁₋₆ alkylamino, morpholin-4-yl, piperidin-1-yl, pyrrolidin-1-yl, or 4-C₁₋₄ alkylpiperazin-1-yl.

### Pharmaceutical Compositions and Biological Activity

Pharmaceutical compositions of the invention contain a therapeutically effective amount of a compound or salt of the invention as described above in combination with a pharmaceutically acceptable carrier.

The terms “a therapeutically effective amount” and “effective amount” mean an amount of the compound or salt sufficient to induce a therapeutic or prophylactic effect, such as cytokine induction, immunomodulation, antitumor activity, and/or antiviral activity. Although the exact amount of active compound or salt used in a pharmaceutical composition of the invention will vary according to factors known to those of skill in the art, such as the physical and chemical nature of the compound or salt, the nature of the carrier, and the intended dosing regimen, it is anticipated that the compositions of the invention will contain sufficient active ingredient to provide a dose of about 100 nanograms per kilogram (ng/kg) to about 50 milligrams per kilogram (mg/kg), preferably about 10 micrograms per kilogram (μg/kg) to about 5 mg/kg, of the compound or salt to the subject. A variety of dosage forms may be used, such as tablets, lozenges, capsules, parenteral formulations, syrups, creams, ointments, aerosol formulations, transdermal patches, transmucosal patches and the like.

The compounds or salts of the invention can be administered as the single therapeutic agent in the treatment regimen, or the compounds or salts of the invention may be administered in combination with one another or with other active agents, including additional immune response modifiers, antivirals, antibiotics, antibodies, proteins, peptides, oligonucleotides, etc.

Compounds or salts of the invention have been shown to induce the production of certain cytokines in experiments performed according to the test set forth below. These results indicate that the compounds or salts are useful as immune response modifiers that can modulate the immune response in a number of different ways, rendering them useful in the treatment of a variety of disorders.

Cytokines whose production may be induced by the administration of compounds or salts of the invention generally include interferon-α (IFN-α) and/or tumor necrosis factor-α (TNF-α) as well as certain interleukins (IL). Cytokines whose biosynthesis may be induced by compounds or salts of the invention include IFN-α, TNF-α, IL-1, IL-6, IL-10 and IL-12, and a variety of other cytokines. Among other effects, these and other cytokines can inhibit virus production and tumor cell growth, making the compounds or salts useful in the treatment of viral diseases and neoplastic diseases. Accordingly, the invention provides a method of inducing cytokine biosynthesis in an animal comprising administering an effective amount of a compound or salt or composition of the invention to the animal. The animal to which the compound or salt or composition is administered for induction of cytokine biosynthesis may have a disease as described infra, for example a viral disease or a neoplastic disease, and administration of the compound or salt may provide therapeutic treatment. Alternatively, the compound or salt may be administered to the animal prior to the animal acquiring the disease so that administration of the compound or salt may provide a prophylactic treatment.

In addition to the ability to induce the production of cytokines, compounds or salts of the invention can affect other aspects of the innate immune response. For example, natural killer cell activity may be stimulated, an effect that may be due to cytokine induction. The compounds or salts may also activate macrophages, which in turn stimulate secretion of nitric oxide and the production of additional cytokines. Further, the compounds or salts may cause proliferation and differentiation of B-lymphocytes.

Compounds or salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the compounds or salts.

Whether for prophylaxis or therapeutic treatment of a disease, and whether for effecting innate or acquired immunity, the compound or salt or composition may be administered alone or in combination with one or more active components as in, for example, a vaccine adjuvant. When administered with other components, the compound or salt and other component or components may be administered separately; together but independently such as in a solution; or together and associated with one another such as (a) covalently linked or (b) non-covalently associated, e.g., in a colloidal suspension.

Conditions for which compounds or salts identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, a compound or salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Compounds or salts of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a compound or salt of the invention to the animal.

An amount of a compound or salt effective to induce cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) over a background level of such cytokines. The precise amount will vary according to factors known in the art but is expected to be a dose of about 100 ng/kg to about 50 mg/kg, preferably about 10 μg/kg to about 5 mg/kg. The invention also provides a method of treating a viral infection in an animal and a method of treating a neoplastic disease in an animal comprising administering an effective amount of a compound or salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a compound or salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg.

In addition to the formulations and uses described specifically herein, other formulations, uses, and administration devices suitable for compounds of the present invention are described in, for example, International Publication Nos. WO 03/077944 and WO 02/036592, U.S. Pat. No. 6,245,776, and U.S. Publication Nos. 2003/0139364, 2003/185835, 2004/0258698, 2004/0265351, 2004/076633, and 2005/0009858.

Objects and advantages of this invention are further illustrated by the following examples, but the particular materials and amounts thereof recited in these examples, as well as other conditions and details, should not be construed to unduly limit this invention.

### EXAMPLES

### Example 1

### 2-(Ethoxymethyl)-N¹-isopropyl-6,7-dimethyl-1H-imidazo[4,5-c]pyridine-1,4-diamine

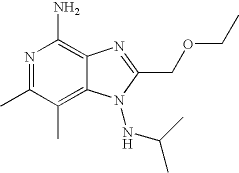

### Part A

tert-Butyl carbazate (23.9 g, 181 mmol) was added to a stirred solution of 2,4-dichloro-5,6-dimethyl-3-nitropyridine (20.0 g, 90.5 mmol) and triethylamine (25.2 mL, 181 mmol) in N,N-dimethylformamide (DM) (200 mL) at room temperature. The reaction was heated for 3 days at 55-70° C. The reaction was allowed to cool to room temperature and then was poured into water (1.8 L), which caused a light brown solid precipitate to form. The solid was isolated by filtration, dried, and recrystallized from toluene to provide 17.5 g of tert-butyl 2-(2-chloro-5,6-dimethyl-3-nitropyridin-4-yl)hydrazinecarboxylate as tan needles.

### Part B

A solution of tert-butyl 2-(2-chloro-5,6-dimethyl-3-nitropyridin-4-yl)hydrazinecarboxylate (15.1 g, 47.8 mmol) and triethylamine (8.60 mL, 62.0 mmol) in dibenzylamine (150 mL) was heated at 70° C. for two nights, then was partitioned between dichloromethane (500 mL) and water (500 mL). The organic layer was filtered and concentrated under reduced pressure to yield an orange oil. The oil was poured onto hexanes (1 L) and a solid precipitated. The solid was isolated by filtration and discarded, and the filtrate was concentrated. The product was purified by flash chromatography (silica gel, gradient elution with 0-30% ethyl acetate in hexanes) followed by recrystallization from hexanes twice to provide 4.30 g of tert-butyl 2-[2-(dibenzylamino)-5,6-dimethyl-3-nitropyridin-4-yl]hydrazinecarboxylate as a yellow solid.

### Part C

A mixture of tert-butyl 2-[2-(dibenzylamino)-5,6-dimethyl-3-nitropyridin-4-yl]hydrazinecarboxylate (0.15 g, 0.31 mmol) and 5% platinum on carbon (30 mg) in toluene (10 mL) was hydrogenated on a Parr apparatus at 50 psi (3.5×10⁵ Pa) for 16 hours. The vessel was flushed with nitrogen gas, and additional tert-butyl 2-[2-(dibenzylamino)-5,6-dimethyl-3-nitropyridin-4-yl]hydrazinecarboxylate (4.05 g, 9.04 mmol), 5% platinum on carbon (0.40 g), and toluene (40 mL) were added to the vessel. The mixture was hydrogenated on a Parr apparatus at 50 psi (3.5×10⁵ Pa) for 16 hours. The mixture was filtered through CELITE filter agent. The filtrate was concentrated under reduced pressure to yield 3.90 g of tert-butyl 2-[3-amino-2-(dibenzylamino)-5,6-dimethylpyridin-4-yl]hydrazinecarboxylate as a pale purple oil.

### Part D

A solution of ethoxyacetyl chloride (1.07 g, 8.71 mmol) in dichloromethane (20 mL) was added to a solution of the material from Part C (3.90 g, 8.71 mmol) and triethylamine (1.21 mL, 8.71 mmol) in dichloromethane (100 mL) at 0° C. The reaction was stirred for 2 hours, then was allowed to warm to room temperature. After two days, water (100 mL) was added and the mixture was extracted with dichloromethane (2×100 mL). The organic layers were combined, dried over sodium sulfate, filtered, and concentrated under reduced pressure to yield an oil. The oil was triturated with hexanes and dried overnight to provide 4.41 g of tert-butyl 2-{2-(dibenzylamino)-3-[(ethoxyacetyl)amino]-5,6-dimethylpyridin-4-yl}hydrazinecarboxylate as a light purple solid.

### Part E

A solution of tert-butyl 2-{2-(dibenzylamino)-3-[(ethoxyacetyl)amino]-5,6-dimethylpyridin-4-yl}hydrazinecarboxylate (0.10 g, 0.19 mmol) and pyridine hydrochloride (0.10 g) in pyridine (3 mL) was heated at reflux overnight. The reaction was allowed to cool to room temperature and was concentrated under reduced pressure. The resulting brown oil was partitioned between dichloromethane (50 mL) and water (50 mL). The organic layer was dried over magnesium sulfate, filtered, and concentrated under reduced pressure. The crude product was purified by flash chromatography (silica gel, 30% ethyl acetate in hexanes) to provide 0.01 g of tert-butyl 4-(dibenzylamino)-2-(ethoxymethyl)-6,7-dimethyl-1H-imidazo[4,5-c]pyridin-1-ylcarbamate as a clear oil that slowly crystallized. The reaction was repeated on 4.27 g of tert-butyl 2-{2-(dibenzylamino)-3-[(ethoxyacetyl)amino]-5,6-dimethylpyridin-4-yl}hydrazinecarboxylate using the same procedure. The reaction was heated for 1 hour at reflux instead of overnight. The crude product (3.90 g) was used without further purification in the next step.

### Part F

The material from Part E and from another experiment (5.0 g) was combined and 4.2 M HCl in ethanol (50 mL) was added. The reaction mixture was heated at 60° C. for two hours, then was concentrated under reduced pressure to yield an oil. The oil was partitioned between dichloromethane (100 mL) and 5% aqueous sodium carbonate (100 mL). The aqueous layer was extracted with dichloromethane (2×100 mL). The organic layers were combined, dried over sodium sulfate, filtered, and concentrated under reduced pressure. The crude product was purified by flash chromatography (silica gel, eluted with 20% ethyl acetate in hexanes) to obtain 3.3 g of N⁴, N⁴-dibenzyl-2-(ethoxymethyl)-6,7-dimethyl-1H-imidazo[4,5-c]pyridine-1,4-diamine as a pale orange oil that slowly solidified over time.

### Part G

A solution of N⁴, N⁴-dibenzyl-2-(ethoxymethyl)-6,7-dimethyl-1H-imidazo[4,5-c]pyridine-1,4-diamine (3.00 g, 7.22 mmol), 2,2-dimethoxypropane (1.78 mL, 14.4 mmol), and pyridinium p-toluene sulfonate (3.0 g) in acetonitrile (60 mL) was heated at reflux for 6 hours. The solvent was removed under reduced pressure and the residue was partitioned between dichloromethane (100 mL) and 5% aqueous sodium carbonate (100 mL). The aqueous layer was extracted with dichloromethane (2×100 mL). The organic layers were combined, dried over magnesium sulfate, filtered, and concentrated under reduced pressure. The crude product was purified by flash chromatography (silica gel, eluted with 5% methanol in dichloromethane) to provide 2.89 g of N⁴, N⁴-dibenzyl-2-(ethoxymethyl)-6,7-dimethyl-N¹-(1-methylethylidene)-1H-imidazo[4,5-c]pyridine-1,4-diamine as an orange oil.

### Part H

Sodium borohydride (0.67 g) was added slowly to a solution of N⁴, N⁴-dibenzyl-2-(ethoxymethyl)-6,7-dimethyl-N¹-(1-methylethylidene)-1H-imidazo[4,5-c]pyridine-1,4-diamine (2.68 g) in methanol (27 mL). After the reaction was complete, the excess sodium borohydride was quenched with saturated aqueous ammonium chloride (50 mL). The methanol was evaporated under reduced pressure. To the remaining solution was added sodium carbonate and the mixture was extracted with dichloromethane (3×100 mL). The organic layers were combined, dried over magnesium sulfate, filtered, and concentrated under reduced pressure. The crude product was purified by flash chromatography (silica gel, eluted with 3% methanol in dichloromethane) to yield 2.28 g of N⁴,N⁴-dibenzyl-2-(ethoxymethyl)-N¹-isopropyl-6,7-dimethyl-1H-imidazo[4,5-c]pyridine-1,4-diamine as a clear oil that solidified overnight.

### Part I

A mixture of N⁴, N⁴-dibenzyl-2-(ethoxymethyl)-N¹-isopropyl-6,7-dimethyl-1H-imidazo[4,5-c]pyridine-1,4-diamine (2.10 g, 4.59 mmol), 10% palladium on carbon (2.10 g), and ammonium formate (2.29 g, 48.2 mmol) in ethanol (160 mL) and methanol (80 mL) was heated at reflux for 5 hours. The reaction mixture was filtered through CELITE filter agent and concentrated under reduced pressure. The resulting solid was partitioned between 5% aqueous sodium hydroxide (100 mL) and dichloromethane (100 mL). The aqueous layer was extracted with dichloromethane (2×100 mL). The organic layers were combined, dried over magnesium sulfate, filtered, and concentrated under reduced pressure. The crude product was purified by flash chromatography (silica gel, 10% methanol in dichloromethane) to provide 1.08 g of a white solid, which was recrystallized from acetone/water, isolated by filtration, washed with water, and dried. The white needles were dissolved in hot isopropanol (20 mL) and 1 M HCl in diethyl ether (3.5 mL) was added, followed by diethyl ether (50 mL), to form a white solid. The solid was isolated, dissolved in hot water (50 mL), and treated with sodium carbonate (1.5 g). The mixture was stirred for 1 hour and a solid was filtered, washed with water, and dried at 50° C. under vacuum overnight to provide 0.87 g of 2-(ethoxymethyl)-N¹-isopropyl-6,7-dimethyl-1H-imidazo[4,5-c]pyridine-1,4-diamine as a white powder, mp 110-120° C.

¹H NMR (300 MHz, CDCl₃) δ 5.18 (d, J=2.5 Hz 1H), 4.91 (br s, 2H), 4.78 (br s, 2H), 3.61 (q, J=7.0 Hz, 2H), 3.52-3.42 (m, 1H), 2.49 (s, 3H), 2.41 (s, 3H), 1.25 (t, J=7.0 Hz, 3H), 1.08 (d, J=6.2 Hz, 6H);

MS (APCI) m/z 278 (M+H)⁺;

Anal. Calcd for C₁₄H₂₃N₅O.0.50H₂O: C, 58.72; H, 8.45; N, 24.45. Found: C, 58.59; H, 8.69; N, 24.55.

### Example 2

### 2-(Ethoxymethyl)-N¹-isopropyl-7-methyl-1H-imidazo[4,5-c]pyridine-1,4-diamine

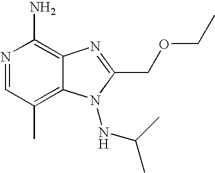

### Part A

Propanenitrile (240 mL, 3.41 mol) was added dropwise to malonyl dichloride (200 g, 1.42 mol). The reaction mixture was stirred for 1 day, during which time a solid formed. Dioxane (600 mL) was added and the solid was isolated by filtration and washed with dioxane (200 mL). The solid was dissolved in hot dioxane (150 mL) and methanol (45 mL). The solution was concentrated to about 150 mL and then was allowed to cool to room temperature, causing a precipitate to form. The solid was isolated by filtration, washed with dioxane, and dried under vacuum at 80° C. to yield 27.5 g of 6-chloro-4-hydroxy-5-methylpyridin-2(1H)-one hydrochloride hydrate.

### Part B

A solution of 6-chloro-4-hydroxy-5-methylpyridin-2(1H)-one hydrochloride hydrate (67.0 g, 0.313 mol) in sulfuric acid (335 mL) was cooled in an ice bath. Nitric acid (19.6 mL, 0.313 mol) was added dropwise over ten minutes. The solution was stirred for 20 minutes, then was poured into ice water (2.5 L). A yellow precipitate formed, was isolated by filtration, and dried under vacuum at 60° C. to provide 39.7 g of 6-chloro-4-hydroxy-5-methyl-3-nitropyridin-2(1H)-one.

### Part C

Triethylamine (20.4 mL, 147 mmol) followed by trifluoromethanesulfonic anhydride (16.4 mL, 97.8 mmol) was added to a solution of 6-chloro-4-hydroxy-5-methyl-3-nitropyridin-2(1H)-one (10.0 g, 48.9 mmol) in dichloromethane (350 mL) at 0° C. solution. The solution was stirred for three hours, then tert-butyl carbazate (7.11 g, 53.8 mmol) was added and the solution was allowed to warm to room temperature. After 16 hours, the reaction was transferred to a separatory funnel and washed with water (300 mL). The organic layer was dried over magnesium sulfate, filtered, and concentrated under reduced pressure. The crude product was purified by on a HORIZON High-Performance Flash Chromatography (HPFC) instrument (available from Biotage, Inc, Charlottesville, Va., USA) (silica gel, eluted with 30% ethyl acetate in hexanes) to yield 15.2 g of tert-butyl 2-(2-chloro-3-methyl-5-nitro-6-{[(trifluoromethyl)sulfonyl]oxy}pyridin-4-yl)hydrazinecarboxylate as a pale orange solid.

### Part D

A solution of tert-butyl 2-(2-chloro-3-methyl-5-nitro-6-{[(trifluoromethyl)sulfonyl]oxy}pyridin-4-yl)hydrazinecarboxylate (15.0 g, 33.3 mmol), triethylamine (4.64 mL, 33.3 mmol), and dibenzylamine (6.40 mL, 33.3 mmol) in toluene (300 mL) was heated at reflux for 6 hours. The reaction was allowed to cool to room temperature, transferred to a separatory funnel, and washed with water (200 mL). The aqueous layer was extracted with toluene (200 mL). The organic layers were combined, dried over magnesium sulfate, filtered, and concentrated under reduced pressure. The resulting oil was purified by HPFC (silica gel, eluted with 20% ethyl acetate in hexanes) to yield 10.9 g of tert-butyl 2-[2-chloro-6-(dibenzylamino)-3-methyl-5-nitropyridin-4-yl]hydrazinecarboxylate as an orange oil.

### Part E

Sodium borohydride (0.94 g) was added to nickel(II) chloride hexahydrate (2.79 g, 11.7 mmol) in methanol (255 mL). A black precipitate formed and after 15 minutes a solution of tert-butyl 2-[2-chloro-6-(dibenzylamino)-3-methyl-5-nitropyridin-4-yl]hydrazinecarboxylate (11.7 g, 23.5 mmol) in methanol (125 mL) and dichloromethane (86 mL) was added. Additional sodium borohydride (0.66 g) was slowly added over ten minutes. Additional sodium borohydride was added over 1 hour until the reaction was complete. The mixture was filtered through CELITE filter agent, and the filter agent was washed with methanol. The filtrate was concentrated under reduced pressure and the crude product was purified by flash chromatography (silica gel, eluted with 10% methanol in dichloromethane) to provide 10.3 g of tert-butyl 2-[3-amino-6-chloro-2-(dibenzylamino)-5-methylpyridin-4-yl]hydrazinecarboxylate was a dark oil that contained an impurity, but was used without further purification in the next step.

### Part F

A solution of ethoxyacetyl chloride (1.85 g, 15.1 mmol) in dichloromethane (50 mL) was added to a solution of the material from Part D (10.1 g, 21.6 mmol) and triethylamine (2.11 mL, 15.1 mmol) in dichloromethane (250 mL) at 0° C. The reaction was stirred for 1 hour, then additional ethoxyacetyl chloride (0.3 equivalent) was added and the solution was allowed to warm to room temperature. The solution was transferred to a separatory funnel and washed with water (100 mL). The organic layer was dried over sodium sulfate, filtered, and concentrated under reduced pressure to yield an oil. The crude product was purified by HPFC (silica gel, eluted with 40% ethyl acetate in hexanes) to provide 7.30 g of tert-butyl 2-{2-chloro-6-(dibenzylamino)-5-[(ethoxyacetyl)amino]-3-methylpyridin-4-yl}hydrazinecarboxylate as a white solid.

### Part G

The method described in Part E of Example 1 was used to convert tert-butyl 2-{2-chloro-6-(dibenzylamino)-5-[(ethoxyacetyl)amino]-3-methylpyridin-4-yl}hydrazinecarboxylate (7.2 g) into 6.7 g of a mixture of tert-butyl 6-chloro-4-(dibenzylamino)-2-(ethoxymethyl)-7-methyl-1H-imidazo[4,5-c]pyridin-1-ylcarbamate and N⁴, N⁴-dibenzyl-6-chloro-2-(ethoxymethyl)-7-methyl-1H-imidazo[4,5-c]pyridine-1,4-diamine. The crude product was used directly in the next step.

### Part H

The method described in Part F of Example 1 was used to convert the material from Part G (6.6 g) into N4, N⁴-dibenzyl-6-chloro-2-(ethoxymethyl)-7-methyl-1H-imidazo[4,5-c]pyridine-1,4-diamine. The crude product was purified by flash chromatography (silica gel, eluted with 30% ethyl acetate/hexanes) to obtain 3.5 g of N⁴, N⁴-dibenzyl-6-chloro-2-(ethoxymethyl)-7-methyl-1H-imidazo[4,5-c]pyridine-1,4-diamine as a pale orange oil that slowly solidified over time, mp 127-129° C.

### Part I

The method described in Part G of Example 1 was used to convert N⁴, N⁴-dibenzyl-6-chloro-2-(ethoxymethyl)-7-methyl-1H-imidazo[4,5-c]pyridine-1,4-diamine (3.30 g) into N⁴, N⁴-dibenzyl-6-chloro-2-(ethoxymethyl)-7-methyl-N¹-(1-methylethylidene)-1H-imidazo[4,5-c]pyridine-1,4-diamine. The crude product was purified by flash chromatography (silica gel, eluted with 40% ethyl acetate/hexanes) to provide 3.50 g of N⁴, N⁴-dibenzyl-6-chloro-2-(ethoxymethyl)-7-methyl-N¹-(1-methylethylidene)-1H-imidazo[4,5-c]pyridine-1,4-diamine as an orange oil.

### Part J

The method described in Part H of Example 1 was used to convert N⁴,N⁴-dibenzyl-6-chloro-2-(ethoxymethyl)-7-methyl-N¹-(1-methylethylidene)-1H-imidazo[4,5-c]pyridine-1,4-diamine (3.40 g, 7.14 mmol) into 3.0 g of N⁴, N⁴-dibenzyl-6-chloro-2-(ethoxymethyl)-N¹-isopropyl-7-methyl-1H-imidazo[4,5-c]pyridine-1,4-diamine as a pale amber oil.

### Part K

The method described in Part I of Example 1 was used to convert N⁴,N⁴-dibenzyl-6-chloro-2-(ethoxymethyl)-N¹-isopropyl-7-methyl-1H-imidazo[4,5-c]pyridine-1,4-diamine (2.90 g, 7.14 mmol) into 1.00 g of 2-(ethoxymethyl)-N¹-isopropyl-7-methyl-1H-imidazo[4,5-c]pyridine-1,4-diamine as a white powder, mp 119-121° C.

¹H NMR (300 MHz, CDCl₃) δ 7.58 (q, J=0.9 Hz, 1H), 5.15 (d, J=2.7 Hz, 1H), 4.97 (br s, 2H), 4.81 (s, 2H), 3.63 (q, J=7.0 Hz, 2H), 3.55-3.44 (m, 1H), 2.51 (d, J=0.9 Hz, 3H), 1.26 (t, J=7.0 Hz, 3H), 1.09 (d, J=6.4 Hz, 6H);

MS (APCI) m/z 264 (M+H)⁺;

Anal. Calcd for C₁₃H₂₁N₅O: C, 59.29; H, 8.04; N, 26.59. Found: C, 58.98; H, 8.21; N, 26.83.

### Example 3

### 2-(Ethoxymethyl)-N¹-isopropyl-6-methyl-1H-imidazo[4,5-c]pyridine-1,4-diamine

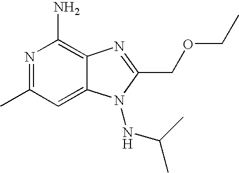

### Part A

Trifluoromethanesulfonic anhydride (29.7 mL, 176 mmol) was added dropwise to a 0° C. solution of 4-hydroxy-6-methyl-3-nitropyridin-2(1H)-one (15.0 g, 88.0 mmol) and triethylamine (36.9 mL, 265 mmol) in dichloromethane (450 mL). The solution was allowed to warm to room temperature and was stirred for two hours before tert-butyl carbazate (12.8 g, 97.0 mmol) was added. The solution was allowed to stir overnight, then was transferred to a separatory funnel and washed with water (200 mL). The organic layer was dried over sodium sulfate, filtered, and concentrated under reduced pressure. The crude product was purified by flash chromatography (silica gel, 30% ethyl acetate in hexanes) to provide 16.2 g of tert-butyl 2-(6-methyl-3-nitro-2-{[(trifluoromethyl)sulfonyl]oxy}pyridin-4-yl)hydrazinecarboxylate as a light orange solid.

### Part B

A solution of dibenzylamine (7.4 mL, 38.7 mmol), tert-butyl 2-(6-methyl-3-nitro-2-{[(trifluoromethyl)sulfonyl]oxy}pyridin-4-yl)hydrazinecarboxylate (16.1 g, 38.7 mmol), and triethylamine (5.4 mL, 38.7 mmol) in toluene (250 mL) was heated at reflux for 6 hours. The reaction mixture was allowed to cool to room temperature and was transferred to a separatory funnel and washed with water (200 mL). The organic layer was dried over sodium sulfate, filtered, and concentrated, and purified by flash chromatography (silica gel, elution with 30% ethyl acetate in hexanes) to yield 17.2 g of tert-butyl 2-[2-(dibenzylamino)-6-methyl-3-nitropyridin-4-yl]hydrazinecarboxylate as an orange solid, mp. 60-67° C.

### Part C

A mixture of tert-butyl 2-[2-(dibenzylamino)-6-methyl-3-nitropyridin-4-yl]hydrazinecarboxylate (17.0 g, 36.7 mmol) and 5% platinum on carbon (1.7 g) in toluene (125 mL) was hydrogenated on a Parr apparatus at 50 psi (3.5×10⁵ Pa) for 16 hours. The vessel was flushed with nitrogen gas, and additional 5% platinum on carbon (2 g) was added to the vessel. The mixture was hydrogenated on a Parr apparatus at 50 psi (3.5×10⁵ Pa) for 4 hours. Additional 5% platinum on carbon (2 g) was added again and the mixture was hydrogenated at 50 psi (3.5×10⁵ Pa) for 4 hours. The mixture was filtered through CELITE filter agent. The filter agent was washed with methanol and dichloromethane. The filtrate was concentrated under reduced pressure to yield 15.9 g of tert-butyl 2-[3-amino-2-(dibenzylamino)-6-methylpyridin-4-yl]hydrazinecarboxylate as a dark amber oil.

### Part D

A solution of ethoxyacetyl chloride (4.90 g, 40.3 mmol) in dichloromethane (100 mL) was added to a solution of tert-butyl 2-[3-amino-2-(dibenzylamino)-6-methylpyridin-4-yl]hydrazinecarboxylate (15.9 g, 36.7 mmol) and triethylamine (5.6 mL) in dichloromethane (350 mL) at 0° C. The reaction was stirred for 1 hour at 0° C., then was allowed to warm to room temperature. After four hours, the solution was transferred to a separatory funnel and was washed with water (200 mL). The organic layer was dried over sodium sulfate, filtered, and concentrated under reduced pressure to yield an oil that was dissolved in pyridine (200 mL). Pyridine hydrochloride (8.0 g) was added to the solution, which was then heated at reflux for 16 hours. The solvent was removed under reduced pressure and the resulting residue was partitioned between 10% aqueous sodium carbonate solution (150 mL) and dichloromethane (150 mL). The aqueous layer was extracted with dichloromethane (2×150 mL). The organic layers were combined, dried over sodium sulfate, filtered, and concentrated under reduced pressure. The crude product was purified by flash chromatography (silica gel, 20% ethyl acetate in hexanes) to provide 13.7 g of tert-butyl 4-(dibenzylamino)-2-(ethoxymethyl)-6-methyl-1H-imidazo[4,5-c]pyridin-1-ylcarbamate as an orange solid, mp. 139-141° C.

### Part E

A solution of tert-butyl 4-(dibenzylamino)-2-(ethoxymethyl)-6-methyl-1H-imidazo[4,5-c]pyridin-1-ylcarbamate (13.6 g, 27.1 mmol) in 2.8 M HCl in ethanol (140 mL) was heated at 65° C. for four hours. The reaction mixture was allowed to cool to room temperature and was concentrated under reduced pressure. The residue was partitioned between dichloromethane and 5% aqueous sodium carbonate. The aqueous layer was extracted with dichloromethane twice. The organic layers were combined, dried over sodium sulfate, filtered, and concentrated under reduced pressure. The crude product was purified by flash chromatography (silica gel, eluted with 40% ethyl acetate/hexanes) to provide 10.7 g of N⁴, N⁴-dibenzyl-2-(ethoxymethyl)-6-methyl-1H-imidazo[4,5-c]pyridine-1,4-diamine as an orange oil.

### Part F

The method used in Part G of Example 1 was used to convert N⁴, N⁴-dibenzyl-2-(ethoxymethyl)-6-methyl-1H-imidazo[4,5-c]pyridine-1,4-diamine (5.00 g, 12.5 mmol) into 4.08 g of N, -dibenzyl-2-(ethoxymethyl)-6-methyl-N¹-(1-methylethylidene)-1H-imidazo[4,5-c]pyridine-1,4-diamine as an orange oil after purification by flash chromatography (silica gel, eluted with 40% ethyl acetate in hexanes).

### Part G

The method used in Part H of Example 1 was used to convert N⁴, N⁴-dibenzyl-2-(ethoxymethyl)-6-methyl-N-(1-methylethylidene)-1H-imidazo[4,5-c]pyridine-1,4-diamine (4.03 g, 9.13 mmol) into 3.44 g of N⁴, N⁴-dibenzyl-2-(ethoxymethyl)-N¹-isopropyl-6-methyl-1H-imidazo[4,5-c]pyridine-1,4-diamine. Additional sodium borohydride was added during the reaction. The crude product was purified by flash chromatography (silica gel, eluted with 2% methanol in chloroform) to provide the product as a pale yellow oil.

### Part H

A mixture of N⁴, N⁴-dibenzyl-2-(ethoxymethyl)-N¹-isopropyl-6-methyl-1H-imidazo[4,5-c]pyridine-1,4-diamine (3.40 g, 7.66 mmol), 10% palladium on carbon (3.4 g), ammonium formate (5.10 g, 80.5 mmol), methanol (15 mL), and ethanol (60 mL) was heated to 85° C. After three hours, additional ammonium formate (1.5 g) was added and the mixture was heated for 3 hours at 85° C., then was allowed to stand at room temperature overnight. More ammonium formate (1.5 g) was added and the reaction was heated for 3 more hours at 85° C. The reaction mixture was allowed to cool to room temperature and was filtered through a plug of CELITE filter agent. The filtrate was concentrated under reduced pressure to yield a white solid that was partitioned between 5% aqueous sodium hydroxide (50 mL) and dichloromethane (50 mL). The aqueous layer was extracted with dichloromethane (2×50 mL). The organic layers were combined, dried over magnesium sulfate, filtered, and concentrated under reduced pressure. The crude product was purified by flash chromatography (silica gel, 10% methanol in chloroform) to provide a solid that was dried under vacuum at 50° C. to yield 1.37 g of 2-(ethoxymethyl)-N¹-isopropyl-6-methyl-1H-imidazo[4,5-c]pyridine-1,4-diamine as a white solid, mp 139-141° C.

¹H NMR (300 MHz, CDCl₃) δ 6.59 (q, J=0.6 Hz, 1H), 5.14 (br s, 2H), 4.97 (d, J=2.4 Hz, 1H), 4.76 (s, 2H), 3.63 (q, J=7.0 Hz, 2H), 3.60 (ds, J=6.3, 2.5 Hz, 1H), 2.46 (d, J=0.6 Hz, 3H), 1.25 (t, J=7.0 Hz, 3H), 1.10 (d, J=6.3 Hz, 6H);

MS (APCI) m/z 264 (M+H)⁺;

Anal. Calcd for C₁₃H₂₁N₅O: C, 59.29; H, 8.04; N, 26.59. Found: C, 59.39; H, 7.95; N, 26.80.

### Example 4

### 2-(Ethoxymethyl)-N¹-isopropyl-1H-imidazo[4,5-c][1,5]naphthyridine-1,4-diamine

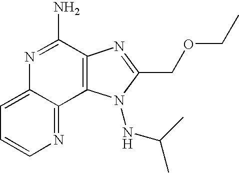

### Part A

A suspension of 4-hydroxy-3-nitro[1,5]naphthyridine (25.0 g, 131 mmol) in 525 mL of DMF was placed under an atmosphere of nitrogen. The reaction flask was placed in an ambient temperature water bath. The suspension was treated with phosphorousoxy chloride (15.8 mL, 170 mmol) at a rate to keep the reaction temperature under 30° C. (total addition time 20 minutes). After 3 hours, the reaction mixture was poured into 1 L of ice water and stirred vigorously until the ice melted. A light yellow precipitate was collected by vacuum filtration and dried for 30 minutes on the filter. The solid was dissolved in dichloromethane (CH₂Cl₂) (750 mL) and dried over MgSO₄. The mixture was filtered through CELITE filter agent and concentrated under reduced pressure to yield 24.6 g of 4-chloro-3-nitro[1,5]naphthyridine as a light yellow solid.

### Part B

A solution of tert-butyl carbazate (16.3 g, 132 mmol) in CH₂Cl₂ (500 mL) was placed under an atmosphere of nitrogen. The solution was chilled in an ice water bath and treated with triethylamine (32.6 mL, 234 mmol). Small portions of 4-chloro-3-nitro[1,5]naphthyridine (24.6 g, 117 mmol) were added to the solution over 10 minutes. The reaction was allowed to slowly come to ambient temperature. After 16 hours, the mixture was concentrated under reduced pressure to yield an orange solid. The solid was dissolved/suspended in chloroform (CHCl₃) (400 mL) and washed with 50% saturated NaHCO₃ solution (2×100 mL). The organic portion was dried over Na₂SO₄, filtered and concentrated under reduced pressure to give tert-butyl 2-(3-nitro[1,5]naphthyridin-4-yl)hydrazinecarboxylate (26.0 g) as a bright orange solid.

### Part C

A suspension of tert-butyl 2-(3-nitro[1,5]naphthyridin-4-yl)hydrazinecarboxylate (10.00 g, 32.75 mmol) in 250 mL of methanol (MeOH) and 250 mL of acetonitrile (MeCN) was treated with platinum (1.00 g, 0.256 mmol, 5% w/w on carbon) and placed under an atmosphere of hydrogen (3.8×10⁵ Pa) and shaken at ambient temperature. After 5 hours, the catalyst was removed by passing the mixture through a pad of CELITE filter agent and rinsing with 1:1 MeOH:MeCN until the filtrate ran clear. The filtrate was concentrated under reduced pressure to yield tert-butyl 2-(3-amino[1,5]naphthyridin-4-yl)hydrazinecarboxylate (9.00 g) as an orange solid.

### Part D

A solution of tert-butyl 2-(3-amino[1,5]naphthyridin-4-yl)hydrazinecarboxylate (9.00 g, 32.7 mmol) in CH₂Cl₂ (100 mL) was placed under an atmosphere of nitrogen and treated with triethylamine (9.12 mL, 65.4 mmol). The solution was chilled in an ice water bath and then slowly treated with ethoxyacetyl chloride (3.69 mL, 34.3 mmol). The reaction was allowed to slowly warm to ambient temperature. After 2 hours, the reaction mixture was concentrated under reduced pressure to give an orange solid. The solid was dissolved in ethanol (100 mL), treated with triethylamine (13.7 mL, 98.1 mmol) and placed under an atmosphere of nitrogen. The reaction was heated to 100° C. After 18 hours, the temperature of the heating bath was raised to 120° C. After an additional 24 hours, the reaction mixture was cooled to ambient temperature and concentrated under reduced pressure to yield a brown semi-solid. The material was suspended in toluene (150 mL) and treated with pyridine hydrochloride (1.0 g, 8.7 mmol). The reaction mixture was placed under an atmosphere of nitrogen and heated to 130° C. After 1 day, the reaction was cooled to ambient temperature and concentrated under reduced pressure to yield a brown solid. The solid was purified using HPFC (silica gel, eluted with 10-25% of a solution comprised of 80:18:2 chloroform:methanol:conc. ammonium hydroxide (CMA) in chloroform) to yield tert-butyl 2-(ethoxymethyl)-1H-imidazo[4,5-c][1,5]naphthyridin-1-ylcarbamate (6.3 g) as a light brown solid.

### Part E

A suspension of tert-butyl 2-(ethoxymethyl)-1H-imidazo[4,5-c][1,5]naphthyridin-1-ylcarbamate (6.30 g, 18.3 mmol) in 2.2 M HCl (120 mL) in ethanol was heated to 100° C. After 45 minutes, the reaction was cooled to ambient temperature and concentrated under reduced pressure to yield a brown solid. The solid was treated with 100 mL of 5% NaOH solution. The solution was concentrated under reduced pressure to give a tan solid residue. The residue was placed in a cellulose extraction thimble and extracted with CH₂Cl₂ (200 mL) using a Soxhlet apparatus. After 6 hours, the CH₂Cl₂ was concentrated under reduced pressure to yield 2-(ethoxymethyl)-1H-imidazo[4,5-c][1,5]naphthyridin-1-amine (2.48 g) as a gray solid.

### Part F

A suspension of 2-(ethoxymethyl)-1H-imidazo[4,5-c][1,5]naphthyridin-1-amine (2.48 g, 10.2 mmol) in MeCN (25 mL) was placed under an atmosphere of nitrogen and treated with glacial acetic acid (5 mL) to give a brown solution. The solution was treated with 2,2-dimethoxy propane (12.5 mL, 102 mmol). The reaction heated to 100° C. After 18 hours, the reaction was cooled to ambient temperature and concentrated under reduced pressure to yield a brown oil. The oil was partitioned between CHCl₃ (50 mL) and 10% aqueous Na₂CO₃ solution (50 mL) and separated. The aqueous portion was extracted with CHCl₃ (2×25 mL). The combined organic portions were washed with water (25 mL) and brine (25 mL). The organic portion was dried over Na₂SO₄, filtered and concentrated under reduced pressure to yield a light brown oil. The oil was purified by HPFC (silica gel, 10% CMA in CHCl₃) to yield 2-(ethoxymethyl)-N-(1-methylethylidene)-1H-imidazo[4,5-c][1,5]naphthyridin-1-amine (2.48 g) as an orange oil.

### Part G

A solution of 2-(ethoxymethyl)-N-(1-methylethylidene)-1H-imidazo[4,5-c][1,5]naphthyridin-1-amine (2.48 g, 8.75 mmol) in methanol (75 mL) was placed under an atmosphere of nitrogen and chilled in an ice water bath. The solution was treated with sodium borohydride (0.99 g, 26.3 mmol) over 3 minutes. The reaction was allowed to warm to ambient temperature. After 72 hours, the reaction was quenched with 15 mL of saturated aqueous ammonium chloride solution. The reaction mixture was concentrated under reduced pressure to yield a yellow solid. The solid was partitioned between CHCl₃ (75 mL) and 10% aqueous Na₂CO₃ solution (25 mL) and separated. The organic portion was washed with H₂O (25 mL) and brine (25 mL), dried over Na₂SO₄, filtered and concentrated under reduced pressure to yield 2-(ethoxymethyl)-N-isopropyl-1H-imidazo[4,5-c][1,5]naphthyridin-1-amine (2.27 g) as yellow crystals.

### Part H

A solution of 2-(ethoxymethyl)-N-isopropyl-1H-imidazo[4,5-c][1,5]naphthyridin-1-amine (2.27 g) in CHCl₃ (75 mL) was treated with 3-chloroperoxybenzoic acid (3.77 g, 10.9 mmol, 50%) over 5 minutes. After 2 hours, the reaction was treated with 30% NH₄OH solution (25 mL) and stirred vigorously. The mixture was treated with p-toluenesulfonyl chloride (1.59 g, 8.36 mmol). After 30 minutes, the reaction mixture was transferred to a separatory funnel and the phases separated. The organic portion was washed with 10% Na₂CO₃ solution (25 mL) and H₂O (25 mL). The combined aqueous washes were back-extracted with CHCl₃ (25 mL). The combined organic portions were washed with brine (25 mL), dried over Na₂SO₄, filtered and concentrated under reduced pressure to give a yellow solid. The solid was purified by HPFC (silica gel, 10% CMA in CHCl₃) to give a light yellow solid. The solid was recrystallized from MeCN to yield 2-(ethoxymethyl)-N¹-isopropyl-1H-imidazo[4,5-c][1,5]naphthyridine-1,4-diamine (0.86 g) as light yellow needle-like crystals, mp 150-152° C.

¹H NMR (300 MHz, DMSO-d₆) δ 8.55 (dd, J=4.3, 1.5 Hz, 1H), 7.93 (dd, J=6.4, 1.6 Hz, 1H), 7.47 (dd, J=8.4, 4.4 Hz, 1H), 6.96 (s, 2H), 6.72 (d, J=2.5 Hz, 1H), 4.75 (s, 2H), 3.89-3.80 (m, 1H), 3.63 (q, J=7.0 Hz, 2H), 1.16 (t, J=7.0 Hz, 3H), 1.02 (d, J=5.9 Hz, 6H);

¹³C NMR (75 MHz, DMSO-d₆) δ 152.7, 151.0, 143.8, 140.4, 133.5, 132.9, 131.7, 127.7, 122.6, 65.9, 62.5, 51.8, 20.4, 15.4;

MS (APCI) m/z 301 (M+H)⁺;

Anal. Calcd for C₁₅H₂₀N₆O: C, 59.98; H, 6.71; N, 27.98; Found: C, 60.11; H, 6.90; N, 28.21.

### Example 5

### 2-(Ethoxymethyl)-N¹-isopropyl-6,7,8,9-tetrahydro-1H-imidazo[4,5-c][1,5]naphthyridine-1,4-diamine

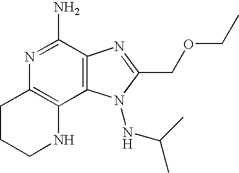

A mixture of 2-(ethoxymethyl)-N¹-isopropyl-1H-imidazo[4,5-c][1,5]naphthyridine-1,4-diamine (0.300 g, 0.100 mmol) and platinum(IV) oxide (0.227 g, 0.100 mmol) in trifluoroacetic acid (15 mL) was hydrogenated on a Parr apparatus at 50 psi (3.4×10⁵ Pa) at room temperature for 15 hours. The mixture was diluted with chloroform (45 mL) and filtered through a pad of CELITE filter agent. The filter agent was rinsed with a 4:1 chloroform/methanol solution. The filtrate was concentrated under reduced pressure to yield an oil that was suspended in water (15 mL) and treated with 50% aqueous sodium hydroxide until pH 13 was reached. The mixture was extracted with dichloromethane (3×15 mL). The organic layers were combined, washed with brine (15 mL), dried over sodium sulfate, filtered, and concentrated under reduced pressure to yield an orange solid. The crude product was purified by HPFC (silica gel, gradient elution with 5-15% CMA in chloroform) to provide approximately 100 mg of 2-(ethoxymethyl)-N¹-isopropyl-6,7,8,9-tetrahydro-1H-imidazo[4,5-c][1,5]naphthyridine-1,4-diamine as an orange solid.

¹H NMR (300 MHz, CDCl₃) δ 5.45 (d, J=3.1, 11H), 4.78-4.75 (m, 3H), 4.62 (br s, 2H), 3.59-3.50 (m, 1H), 3.57 (q, J=7.0, 2H), 3.30-3.26 (m, 2H), 2.86 (t, 2H), 2.09-2.01 (m, 2H), 1.23 (t, J=7.0, 3H), 1.11 (d, J=6.4, 6H);

MS (APCI) m/z 305 (M+H)⁺;

### Example 6

### [4-Amino-1-(isopropylamino)-1H-imidazo[4,5-c][1,5]naphthyridin-2-yl]methanol

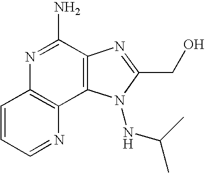

Under a nitrogen atmosphere boron tribromide (2.33 mL of 1 M in dichloromethane) was added to a chilled (ice/water bath) solution of 2-ethoxymethyl-N¹-isopropyl-1H-imidazo[4,5-c][1,5]naphthyridine-1,4-diamine (0.350 g, 1.17 mmol) in dichloromethane (10 mL). The reaction mixture was allowed to slowly warm to ambient temperature and then was stirred for 18 hours. The reaction mixture was chilled, treated with additional boron tribromide (2.00 mL), allowed to warm to ambient temperature, and then stirred for 5 hours. The reaction mixture was quenched with methanol, allowed to stir for 4 days, and then concentrated under reduced pressure. The residue was combined with aqueous 6 M hydrochloric acid (25 mL), heated to 50° C., and stirred for 2 hours. The resulting solution was allowed to cool to ambient temperature, the pH was adjusted to 7 with 10% aqueous sodium hydroxide, and the mixture was stirred for 30 minutes. A precipitate was isolated by filtration, washed with water, and then dried to provide a white solid. This material was purified by chromatography (silica gel eluted with 15% CMA in chloroform) to provide 105 mg of [4-amino-1-(isopropylamino)-1H-imidazo[4,5-c][1,5]naphthyridin-2-yl]methanol as a white solid, mp 242-243° C. ¹H NMR (500 MHz, DMSO-d₆) δ 8.54 (dd, J=4.4, 1.4 Hz, 1H), 7.92 (dd, J=8.4, 1.4 Hz, 1H), 7.46 (dd, J=8.4, 4.3 Hz, 1H), 6.86 (s, 2H), 6.64 (d, J=2.7 Hz, 1H), 5.40 (t, J=6.9 Hz, 1H), 4.77 (d, J=5.9 Hz, 2H), 3.87-3.81 (m, 1H), 1.03 (d, J=4.5 Hz, 6H); ¹³C NMR (125 MHz, DMSO-d₆) δ 154.0, 152.7, 143.8, 140.3, 133.6, 132.9, 131.7, 127.6, 122.5, 55.0, 51.9, 20.5; MS (APCI) m/z 273 (M+H)⁺; Anal. calcd for C₁₃H₁₆N₆O: C, 57.34; H, 5.92; N, 30.86; Found: C, 57.20; H, 5.76; N, 31.14.

### Example 7

### 2-Ethyl-N¹-isopropyl-1H-imidazo[4,5-c][1,5]naphthyridine-1,4-diamine

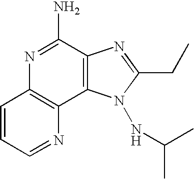

### Part A

Tert-butyl 2-(3-amino[1,5]naphthyridin-4-yl)hydrazinecarboxylate (8.95 g, 32.5 mmol), triethyl orthopropionate (7.20 mL, 35.8 mmol), pyridinium para-toluenesulfonate (0.408 g, 1.63 mmol) and toluene (130 mL) were combined and heated at 120° C. in a flask equipped with a Dean-Stark trap and a condenser. After 4 hours the reaction mixture was allowed to cool to ambient temperature and then it was concentrated under reduced pressure. The residue was dissolved in chloroform (150 mL) and the solution was washed sequentially with 10% aqueous sodium carbonate (45 mL) and brine (45 mL). The aqueous washes were back extracted with chloroform (30 mL). The combined organics were washed with brine (45 mL), dried over sodium sulfate, filtered, and then concentrated under reduced pressure to provide a red/brown foam. This material was dissolved in chloroform and passed through a layer of silica gel (150 g) which was eluted with 9:1 chloroform methanol to provide 9.63 g of tert-butyl 2-ethyl-1H-imidazo[4,5-c][1,5]naphthyridin-1-ylcarbamate.

### Part B

The material from Part A was combined with hydrogen chloride in ethanol (70 mL of 2.2 M), heated to 100° C., stirred for 30 minutes, and then allowed to slowly cool to ambient temperature. The reaction mixture was diluted with diethyl ether (75 mL) and then cooled in an ice/water bath. A precipitate was isolated by filtration and dried to provide a brown solid. This material was dissolved in a minimum amount of water, chilled in an ice/water bath, and then treated with 50% aqueous sodium hydroxide until the pH reached 13. A precipitate was isolated by filtration and dried under vacuum to provide 4.5 g of 2-ethyl-1H-imidazo[4,5-c][1,5]naphthyridin-1-amine as a brown solid.

### Part C

Acetic acid (1.0 mL) was added to a suspension of 2-ethyl-1H-imidazo[4,5-c][1,5]naphthyridin-1-amine (1.00 g, 4.69 mmol) in acetonitrile (10 mL) to provide a solution. 2,2-Dimethoxypropane (1.15 mL, 9.38 mmol) was added and the solution was heated to 100° C. After 7 hours, additional ketal (1 mL) was added and the reaction mixture was heated for an additional 5 hours. The reaction mixture was cooled to ambient temperature and then concentrated under reduced pressure to provide a brown oil. The oil was taken up in chloroform (30 mL) and the solution was washed with 10% aqueous sodium carbonate (2×10 mL). The combined aqueous washes were back extracted with chloroform (20 mL). The combined organics were dried over sodium sulfate and then concentrated under reduced pressure to provide 0.98 g of 2-ethyl-N-(methylethylidene)-1H-imidazo[4,5-c][1,5]naphthyridin-1-amine as a thick brown oil.

### Part D

Under a nitrogen atmosphere sodium borohydride (0.29 g, 7.74 mmol) was added in portions over a period of 2 minutes to a solution of the material from Part C (3.87 mmol) in methanol (16 mL). After 2 hours the reaction mixture was quenched by the slow addition of aqueous saturated ammonium chloride (5 mL) and then concentrated under reduced pressure. The residue was partitioned between chloroform (30 mL) and saturated aqueous sodium bicarbonate (10 mL). The organic was washed sequentially with water (15 mL) and brine (15 mL), dried over sodium sulfate, filtered, and then concentrated under reduced pressure. The residue was purified by HPFC (silica gel eluted with a gradient of 0-50% CMA in chloroform) to provide 0.78 g of 2-ethyl-N-isopropyl-1H-imidazo[4,5-c][1,5]naphthyridin-1-amine as an orange oil.

### Part E

The material from Part D was oxidized and then aminated using the general method of Example 4 Part H. The crude product was purified by HPFC (100 g of silica gel eluted with a gradient of 10-50% CMA in chloroform) to provide a tan foam. This material was recrystallized from acetonitrile and then from n-propyl acetate to provide 44 mg of 2-ethyl-N¹-isopropyl-1H-imidazo[4,5-c][1,5]naphthyridine-1,4-diamine as tan crystals, mp 212-213° C. ¹H NMR (500 MHz, DMSO-d₆) δ 8.53 (dd, J=4.3, 1.3 Hz, I H), 7.90 (dd, J=8.4, 1.3 Hz, 1H), 7.43 (dd, J=8.4, 4.3 Hz, 1H), 6.77 (s, 2H), 6.58 (d, J=2.2 Hz, 1H), 3.86-3.79 (m, 1H), 2.97 (q, J=7.5 Hz, 2H), 1.35 (t, J=7.5 Hz, 3H), 1.02 (bs, 6H); ¹³C NMR (125 MHz, DMSO-d₆) δ 156.4, 152.4, 143.7, 140.0, 133.5, 132.8, 131.6, 127.6, 122.2, 51.8, 20.5, 19.8, 12.4; MS (APCI) m/z 271.14 (M+H)⁺; Anal. calcd for C₁₄H₁₈N₆: C, 62.20; H, 6.71; N, 31.09; Found: C, 61.84; H, 6.47; N, 31.31.

### Example 8

### N-{3-[(4-amino-2-ethyl-1H-imidazo[4,5-c][1,5]naphthyridin-1-yl)amino]propyl}methanesulfonamide

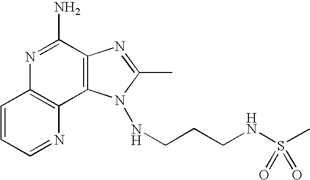

### Part A

Triethylamine (26.1 mL, 187 mmol) was added to a solution of 1-amino-3,3-diethoxypropane (27.5 mL, 170 mmol) in tetrahydrofuran (75 mL). The solution was chilled in an ice/water bath and then a solution of di-tert-butyl dicarbonate (40.8 g, 187 mmol) in tetrahydrofuran (125 mL) was added dropwise over a few hours. The reaction mixture was allowed to slowly warm to ambient temperature and then stirred at ambient temperature. After 15 hours the reaction mixture was concentrated under reduced pressure to provide a yellow oil. The oil was dissolved in ethyl acetate (200 mL). The solution was washed sequentially with water (2×50 mL) and brine (50 mL), dried over sodium sulfate, filtered, and then concentrated under reduced pressure to provide a yellow oil. This material was dried under high vacuum to provide 42.0 g of tert-butyl (3,3-diethyoxypropyl)carbamate.

### Part B

Acetic acid (3.0 mL) was added to a suspension of 2-ethyl-1H-imidazo[4,5-c][1,5]naphthyridin-1-amine (3.00 g, 14.1 mmol) in acetonitrile (30 mL) to provide a solution. tert-Butyl (3,3-diethyoxypropyl)carbamate (3.83 g, 15.5 mmol) was added and the solution was heated to 100° C. After 3 hours additional acetic acid (3 mL) was added. After 16 hours the reaction mixture was cooled to ambient temperature and then concentrated under reduced pressure to provide a brown oil. The oil was partitioned between chloroform (50 mL) and 10% aqueous sodium carbonate (15 mL). The organic was washed with water (2×15 mL). The combined aqueous washes were back extracted with chloroform (15 mL). The combined organics were washed with brine (20 mL), dried over sodium sulfate, filtered, and then concentrated under reduced pressure to provide 4.70 g of tert-butyl 3-[(2-ethyl-1H-imidazo[4,5-c][1,5]naphthyridin-1-yl)imino]propylcarbamate as a brown solid.

### Part C

The material from Part B was reduced using the method of Example 7 Part D. The crude product was purified by HPFC (350 g of silica gel eluted with a gradient of 0-50% CMA in chloroform) to provide 2.75 g of tert-butyl 3-[(2-ethyl-1H-imidazo[4,5-c][1,5]naphthyridin-1-yl)amino]propylcarbamate as an orange foam.

### Part D

3-Chloroperbenzoic acid (1.60 g of 50%, 9.28 mmol) was added to a solution of the material from Part C (7.42 mmol) in chloroform (75 mL) and the reaction mixture was stirred for 1.5 hours. Ammonium hydroxide (25 mL of 30%) was added. Para-Toluenesulfonyl chloride (1.49 g, 7.79 mmol) was added in small portions over a period of 3 minutes with vigorous stirring. The reaction mixture was placed in a warm water bath and stirred for 15 minutes. The reaction mixture was diluted with chloroform (75 mL) and water (25 mL). The organic layer was washed sequentially with aqueous 10% sodium carbonate (50 mL) and brine (50 mL), dried over sodium sulfate, filtered, and then concentrated under reduced pressure to provide >2.86 g tert-butyl 3-[(4-amino-2-ethyl-1H-imidazo[4,5-c][1,5]naphthyridin-1-yl)amino]propylcarbamate as a yellow foam.

### Part E

The tert-butoxycarbonyl group was removed from the material from Part D using the method of Example 7 Part B. The crude product was recrystallized from a mixture of acetonitrile and methanol to provide 0.82 g of N¹-(3-aminopropyl)-2-ethyl-1H-imidazo[4,5-c][1,5]naphthyridine-1,4-diamine as a light yellow solid.

### Part F

Triethylamine (1.20 mL, 8.7 mmol) was added to a suspension of the material from Part E (2.9 mmol) in dichloromethane (15 mL). The reaction mixture was chilled in an ice/water bath and then methanesulfonyl chloride (0.25 mL, 3.2 mmol) was added dropwise. The reaction mixture was stirred under nitrogen and allowed to warm to ambient temperature overnight. The reaction mixture was concentrated under reduced pressure to provide a yellow/orange solid. This material was purified by HPFC (silica gel eluted with a gradient of 15-65% CMA in chloroform) to provide a yellow solid. This solid was recrystallized from acetonitrile and then dried at 100° C. to provide 495 mg of N-{3-[(4-amino-2-ethyl-1H-imidazo[4,5-c][1,5]naphthyridin 1-yl)amino]propyl}methanesulfonamide as a yellow crystals, mp 176-178° C.; ¹H NMR (500 MHz, DMSO-d₆) δ 8.55 (dd, J=4.3, 1.1 Hz, 1H), 7.92 (dd, J=8.4, 1.3 Hz, 1H), 7.46 (dd, J=8.4, 4.4 Hz, 1H), 7.01 (t, J=5.8 Hz, 1H), 6.80 (s, 2H), 6.73 (t, J=5.9 Hz, 1H), 3.31-3.28 (m, 2H), 3.11 (q, J=6.7 Hz, 2H), 2.97 (q, J=7.5 Hz, 2H), 2.89 (s, 3H), 1.73 (p, J=7.0 Hz, 2H), 1.36 (t, J=7.5 Hz, 3H); ¹³C NMR (125 MHz, DMSO-d₆) δ 155.6, 152.4, 143.7, 140.0, 133.3, 132.8, 131.4, 127.4, 122.3, 50.1, 40.9, 39.4, 28.1, 19.7, 12.4; MS (APCI) m/z 364.11 (M+H)⁺; Anal. calcd for C₁₅H₂₁N₇O₂S: C, 49.57; H, 5.82; N, 26.98; Found: C, 49.72; H, 5.73; N, 27.06.

### Example 9

### 2-(Ethoxymethyl)-N¹,N¹,6,7-tetramethyl-1H-imidazo[4,5-c]pyridine-1,4-diamine

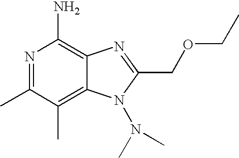

### Part A

2,4-Dichloro-5,6-dimethyl-3-nitropyridine (31.5 g, 143 mmol), triethylamine (30.0 mL, 215 mmol), 1,1-dimethylhydrazine (13.3 mL, 215 mmol) and N,N-dimethylformamide (DMF, 300 mL) were combined and heated at 70° C. for about 170 hours. The reaction mixture was allowed to cool to ambient temperature, diluted with water (700 mL), and then filtered to remove a solid. The filtrate was extracted with ethyl acetate (2×500 mL). The combined extracts were washed sequentially with water (500 mL) and brine (500 mL), dried over sodium sulfate, filtered, and then concentrated under reduced pressure. The residue was purified by HPFC (silica gel eluted with a gradient of 2-100% ethyl acetate in hexanes) and then triturated with ethyl acetate/hexanes. The resulting solid was isolated by filtration and dried to provide 14.63 g of 2-chloro-4-(2,2-dimethylhydrazino)-5,6-dimethyl-3-nitropyridine as pale yellow crystals.

### Part B

2-Chloro-4-(2,2-dimethylhydrazino)-5,6-dimethyl-3-nitropyridine (13.6 g), 5% platinum on carbon (1.55 g of 50% water wet), and toluene (200 mL) were combined an placed under hydrogen pressure on a Parr apparatus for 19 hours. The reaction mixture was filtered through a layer of CELITE filter aid. The filter cake was rinsed with ethanol. The filtrate was concentrated under reduced pressure. The residue was concentrated twice from toluene to provide 2-chloro-4-(2,2-dimethylhydrazino)-5,6-dimethylpyridin-3-amine.

### Part C

Triethylamine (5.4 mL, 38.9 mmol) was added to a chilled (0° C.) solution of 2-chloro-4-(2,2-dimethylhydrazino)-5,6-dimethylpyridin-3-amine (27.8 mmol) in dichloromethane (60 mL). Ethoxyacetyl chloride (3.75 g, 30.6 mmol) was added dropwise. The reaction mixture was allowed to warm to ambient temperature, during which time additional dichloromethane, triethylamine, and ethoxyacetyl chloride were added until the reaction was complete. The reaction mixture was washed sequentially with water and brine and then concentrated under reduced pressure. The residue was dissolved in ethanol (80 mL). A solution of sodium hydroxide (3.34 g, 83.4 mmol) in water (15 mL) was added. The reaction solution was heated at reflux for 1.5 hours, allowed to stand at ambient temperature overnight, and then concentrated under reduced pressure. The residue was partitioned between ethyl acetate and saturated aqueous ammonium chloride. The aqueous layer was extracted with ethyl acetate (2×). The combined organics were washed sequentially with water and brine, dried over sodium sulfate, filtered, and then concentrated under reduced pressure to provide 6.7 g of a tan solid. This material was triturated with ethyl acetate/hexanes and then isolated by filtration to provide 3.59 g of 4-chloro-2-(ethoxymethyl)-N,N,6,7-tetramethyl-1H-imidazo[4,5-c]pyridin-1-amine.

### Part D

Each of 3 tubes was charged with 2,2,2-trifluoroethanol (13 mL), pyridine hydrochloride (2 g), 4-methoxybenzylamine (4.6 mL), and 4-chloro-2-(ethoxymethyl)-N,N,6,7-tetramethyl-1H-imidazo[4,5-c]pyridin-1-amine (1.00 g). Each tube was heated at 160° C. in a microwave for 2 hours. The contents of the tubes were combined and concentrated under reduced pressure. The residue was partitioned between ethyl acetate and saturated aqueous sodium carbonate. The aqueous layer was extracted with ethyl acetate. The combined organics were washed sequentially with water and brine, dried over sodium sulfate, filtered, and then concentrated under reduced pressure. The residue was purified by HPFC (silica gel eluted with a gradient of ethyl acetate in hexanes) to provide 1.47 g of 2-(ethoxymethyl)-N⁴-(4-methoxybenzyl)-N¹,N¹,6,7-tetramethyl-1H-imidazo[4,5-c]pyridine-1,4-diamine.

### Part E

A solution of the material from Part D in trifluoroacetic acid (50 mL) was allowed to stand at ambient temperature for 24 hours and then it was concentrated under reduced pressure. The residue was partitioned between dichloromethane and 10% sodium hydroxide. The aqueous layer was extracted with dichloromethane (×2). The combined organics were washed sequentially with water and brine, dried over sodium sulfate, filtered, and then concentrated under reduced pressure to provide a solid. This material was triturated with cold toluene, isolated by filtration, recrystallized from hot toluene, isolated by filtration, and dried to provide 523 mg of 2-(ethoxymethyl)-N¹,N¹,6,7-tetramethyl-1H-imidazo[4,5-c]pyridine-1,4-diamine as white crystals, mp 170.0-171.0° C. ¹H NMR (CDCl₃) δ 4.87 (br s, 2H), 4.73 (s, 2H), 3.64 (q, J=7.0 Hz, 2H), 3.05 (s, 6H), 2.48 (s, 3H), 2.43 (s, 3H), 1.25 (t, J=7.0, 3H); MS (APCI) m/z 264 (M+H)⁺; Anal. calcd for C₁₃H₂₁N₅O: C, 59.29; H, 8.04; N, 26.59. Found: C, 59.42; H, 7.91; N, 26.66.

### Example 10

### N¹,N¹,6,7-Tetramethyl-1H-imidazo[4,5-c]pyridine-1,4-diamine

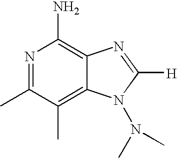

### Part A

A mixture of 2-chloro-4-(2,2-dimethylhydrazino)-5,6-dimethylpyridin-3-amine (27.8 mmol), triethyl orthoformate (6.0 mL, 1.3 eq), pyridine hydrochloride (0.97 g, 0.3 eq), and toluene (60 mL) was heated at reflux for 1.5 hours and then allowed to cool to ambient temperature. The reaction mixture was diluted with ethyl acetate and then washed sequentially with aqueous saturated ammonium chloride, water, and brine. The organic layer was dried over sodium sulfate, filtered, and then concentrated under reduced pressure. The residue was triturated with ethyl acetate/hexanes to provide a solid which was isolated by filtration and then dried to provide 3.97 g of 4-chloro-N,N,6,7-tetramethyl-1H-imidazo[4,5-c]pyridin-1-amine as a pale pink solid.

### Part B

A process vial was charged with 4-chloro-N,N,6,7-tetramethyl-1H-imidazo[4,5-c]pyridin-1-amine (0.5 g), 4-methoxybenzylamine (2.9 mL), pyridine hydrochloride (1.3 g), and 2,2,2-trifluoroethanol (10 mL). The vial was heated at 160° C. for 2 hours in a microwave. The reaction mixture was allowed to cool to ambient temperature and then filtered. The filtrate was concentrated under reduced pressure. The residue was partitioned between ethyl acetate and water. The organic layer was washed sequentially with water and brine, dried over sodium sulfate, filtered, and then concentrated under reduced pressure to provide crude N⁴-(4-methoxybenzyl)-N¹,N¹,6,7-tetramethyl-1H-imidazo[4,5-c]pyridine-1,4-diamine as a red oil.

### Part C

A solution of the material from Part B in trifluoroacetic acid (15 mL) was allowed to stand at ambient temperature for 16 hours and then it was concentrated under reduced pressure. The residue was partitioned between dichloromethane and 10% sodium hydroxide. The aqueous layer was extracted with dichloromethane (×2). The combined organics were washed sequentially with water and brine, dried over sodium sulfate, filtered, and then concentrated under reduced pressure to provide a solid. This material was triturated with toluene twice, isolated by filtration, and dried to provide 304 mg of N¹,N¹,6,7-tetramethyl-1H-imidazo[4,5-c]pyridine-1,4-diamine as a white powder, mp 205.0-207.0° C. ¹H NMR (CDCl₃) δ 8.07 (s, 1H), 4.87 (br s, 2H), 2.99 (s, 6H), 2.51 (s, 3H), 2.42 (s, 3H); MS (APCI) m/z 206 (M+H)⁺; Anal. calcd for C₁₀H₁₅N₅: C, 58.52; H, 7.37; N, 34.12. Found: C, 58.32; H, 7.65; N, 33.83.

### Example 11

### N¹-Isopropyl-2,6,7-trimethyl-1H-imidazo[4,5-c]pyridine-1,4-diamine

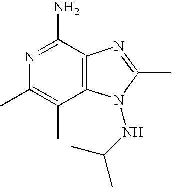

N¹-Isopropyl-2,6,7-trimethyl-1H-imidazo[4,5-c]pyridine-1,4-diamine was prepared accord [symbol] thods of Example 1 using acetyl chloride in lieu of ethoxyacetyl chloride in Part D. The crude product was purified by column chromatography (silica gel eluting with a gradient of 15-30% methanol in dichloromethane) to provide 0.65 g of a white solid. This material was recrystallized from isopropanol (10 mL) to provide 0.49 g of white crystals. This material was dissolved in methanol, concentrated under reduced pressure, and then dried under high vacuum at 60° C. overnight to provide 0.49 g of N¹-isopropyl-2,6,7-trimethyl-1H-imidazo[4,5-c]pyridine-1,4-diamine as a white solid, mp 203-205° C. ¹H NMR (300 MHz, CDCl₃) δ 4.85 (br s, 2H), 4.67 (d, J=1.6 Hz, 1H), 3.43 (ds, J=6.3, 1.6 Hz, 1H), 2.55 (s, 3H), 2.43 (s, 3H), 2.41 (s, 3H), 1.07 (d, J=6.3 Hz, 6H); MS (APCI) m/Z 234 M+H)⁺; Anal. Calcd for C₁₂H₁₉N₅: C, 61.78; H, 8.21; N, 30.02. Found: C, 61.54; H, 8.25; N, 30.18.

### Example 12

### (4-Amino-1-isopropylamino-6,7-dimethyl-1H-imidazo[4,5-c]pyridin-2-yl)methanol

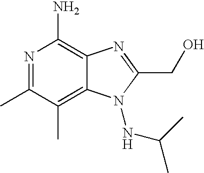

Under a nitrogen atmosphere, boron tribromide (11.3 mL of 1 M in dichloromethane) was added dropwise to a chilled (ice bath) solution of 2-ethoxymethyl-N¹-isopropyl-6,7-dimethyl-1H-imidazo[4,5-c]pyridine-1,4-diamine (1.25 g, 4.51 mmol) in dichloromethane (30 mL). After 1 hour the reaction mixture was allowed to warm to ambient temperature and then it was stirred overnight. The reaction mixture was quenched with methanol (20 mL) and then stirred for 20 minutes. Hydrochloric acid (20 mL of 6 N) was added. The reaction mixture was heated at 40° C. for 2 hours and then let stir at ambient temperature overnight. The pH of the reaction mixture was adjusted to 13 with 50% sodium hydroxide and then it was extracted with dichloromethane (5×100 mL). The combined extracts were concentrated under reduced pressure. The residue was purified by column chromatography (silica gel eluting with a gradient of 20-40% methanol in dichloromethane) to provide 0.7 g of a white solid. This material was recrystallized from ethanol and then dried under vacuum at 80° C. overnight to provide 0.54 g of (4-amino-1-isopropylamino-6,7-dimethyl-1H-imidazo[4,5-c]pyridin-2-yl)methanol as white crystals, mp 257-259° C. ¹H NMR (300 MHz, DMSO-d₆) δ 6.39 (d, J=1.9 Hz, 1H), 5.71 (br s, 2H), 5.43 (br t, J=5.7 Hz, 1H), 4.66 (d, J=5.1 Hz, 2H), 3.43 (m, 1H), 2.43 (s, 3H), 2.27 (s, 3H), 0.95 (d, J=6.2 Hz, 6H); MS (APCI) m/z 250 (M+H)⁺; Anal. Calcd for C₁₂H₁₉N₅O: C, 57.81; H, 7.68; N, 28.09. Found: C, 57.95; H, 7.62; N, 28.27.

### Example 13

### N-{3-[(4-Amino-2-ethoxymethyl-6,7-dimethyl-1H-imidazo[4,5-c]pyridin-1-yl)amino]propyl}methanesulfonamide

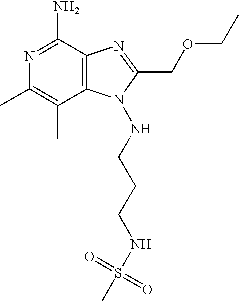

### Part A

A mixture of tert-butyl 2-(2-chloro-5,6-dimethyl-3-nitropyridin-4-yl)hydrazinecarboxylate (20 g), 5% platinum on carbon (4 g), and toluene (200 mL) was hydrogenated on a Parr apparatus at 50 psi (3.5×10⁵ Pa) for 6 hours. The reaction mixture was filtered through a layer of CELITE filter agent. The filter cake was washed with methanol and dichloromethane. The filtrate was concentrated under reduced pressure to provide a light green solid. This material was recrystallized from acetonitrile to provide 14.2 g of tert-butyl 2-(3-amino-2-chloro-5,6-dimethylpyridin-4-yl)hydrazinecarboxylate as white needles.

### Part B

A solution of ethoxyacetyl chloride (6.1 g, 1 eq) in dichloromethane (100 mL) was added dropwise to a chilled (ice bath) solution of tert-butyl 2-(3-amino-2-chloro-5,6-dimethylpyridin-4-yl)hydrazinecarboxylate (14.2 g, 1 eq) and triethylamine (10.4 mL, 1.5 eq) in dichloromethane (900 mL). The reaction mixture was kept cool for 1 hour and then allowed to warm to ambient temperature overnight. Additional ethoxyacetyl chloride (0.3 eq) was added. After 2 hours the reaction mixture was washed with water (100 mL) and then concentrated under reduced pressure. The residue was dissolved in a mixture of ethanol (135 mL) and water (15 mL). Sodium hydroxide (5.9 g, 3 eq) was added and the reaction mixture was heated to reflux. After 1 hour the reaction mixture was diluted with water (400 mL) and then extracted with dichloromethane (3×100 mL). The combined organics were filtered and then concentrated under reduced pressure to provide 15.1 g of tert-butyl 4-chloro-2-ethoxymethyl-6,7-dimethyl-1H-imidazo[4,5-c]pyridin-1-ylcarbamate as an orange frothy solid.

### Part C

A solution of tert-butyl 4-chloro-2-ethoxymethyl-6,7-dimethyl-1H-imidazo[4,5-c]pyridin-1-ylcarbamate (12.80 g) in dichloromethane (100 mL) was added to a chilled (ice bath) solution of trifluoroacetic acid (12 mL) in dichloromethane (150 mL). The reaction mixture was allowed to warm to ambient temperature. After 2 hours additional trifluoroacetic acid (25 mL) was added. The reaction mixture was stirred for an additional 16 hours and then concentrated under reduced pressure. The residue was partitioned between 10% sodium carbonate (200 mL) and dichloromethane (200 mL). The pH of the aqueous layer was adjusted to 14 with sodium hydroxide and then it was extracted with dichloromethane (3×200 mL). The combined organics were dried over sodium sulfate, filtered, and then concentrated under reduced pressure. The residue was triturated with a mixture of ethyl acetate and hexanes to provide 7.7 g of 4-chloro-2-ethoxymethyl-6,7-dimethyl-1H-imidazo[4,5-c]pyridin-1-amine as light orange crystals.

### Part D

Under a nitrogen atmosphere a mixture of 4-chloro-2-ethoxymethyl-6,7-dimethyl-1H-imidazo[4,5-c]pyridin-1-amine (1.0 g, 1 eq), (3,3-diethoxypropyl)carbamic acid (1.07 g, 1.1 eq), pyridinium para-toluenesulfonate (1.0 g), and anhydrous acetonitrile (10 mL) was heated at reflux for 4 hours and then allowed to cool to ambient temperature. The reaction was repeated on a larger scale (6.7 g of tert butyl 4-chloro-2-ethoxymethyl-6,7-dimethyl-1H-imidazo[4,5-c]pyridin-1-amine). The reaction mixtures were combined and then concentrated under reduced pressure. The residue was partitioned between 10% sodium carbonate (100 mL) and dichloromethane (100 mL). The aqueous layer was separated and then extracted with dichloromethane (2×100 mL). The combined organics were concentrated under reduced pressure. The residue was purified by column chromatography (silica gel eluting with 5% methanol in dichloromethane) to provide 9.0 g of tert butyl {3-[(4-chloro-2-ethoxymethyl-6,7-dimethyl-1H-imidazo[4,5-c]pyridin-1-yl)imino]propyl}carbamate as an amber oil.

### Part E

Under a nitrogen atmosphere, sodium borohydride (2.49 g, 3 eq) was slowly added to a solution of the material from Part D (1 eq) in methanol (150 mL). After 3 hours the reaction mixture was quenched with saturated aqueous ammonium chloride (50 mL). The methanol was removed under reduced pressure. Sodium carbonate and dichloromethane (100 mL) were added. The aqueous layer was separated and then extracted with dichloromethane (2×100 mL). The combined organics were dried over magnesium sulfate, filtered, and then concentrated under reduced pressure to provide 8.86 g of tert butyl {3-[(4-chloro-2-ethoxymethyl-6,7-dimethyl-1H-imidazo[4,5-c]pyridin-1-yl)amino]propyl}carbamate as a light orange oil.

### Part F

Under a nitrogen atmosphere trifluoroacetic acid (30 mL) was added dropwise to a chilled (ice bath) solution of the material from Part E in dichloromethane (150 mL). After 1 hour the reaction mixture was allowed to warm to ambient temperature. After 3 hours the reaction mixture was concentrated under reduced pressure. The residue was partitioned between 10% sodium hydroxide (100 mL) and dichloromethane (100 mL). The aqueous layer was separated and then extracted with dichloromethane (2×100 mL). The combined organics were dried over sodium sulfate, filtered, and then concentrated under reduced pressure to provide 6.66 g of N¹-(4-chloro-2-ethoxymethyl-6,7-dimethyl-1H-imidazo[4,5-c]pyridin-1-yl)propane-1,3-diamine as an amber oil.

### Part G

Under a nitrogen atmosphere methanesulfonyl chloride (0.66 g, 1.2 eq) was added dropwise to a chilled (ice bath) solution of N¹-(4-chloro-2-ethoxymethyl-6,7-dimethyl-1H-imidazo[4,5-c]pyridin-1-yl)propane-1,3-diamine (2.2 g, 1 eq) and triethylamine (2.95 mL, 3.0 eq) in dichloromethane (50 mL). After 1 hour the reaction mixture was allowed to warm to ambient temperature and then it was stirred for an additional 2 hours. The reaction mixture was washed with water (50 mL). The aqueous was extracted with dichloromethane (100 mL). The combined organics were concentrated under reduced pressure. The residue was purified by column chromatography (silica gel eluting with 5% methanol in chloroform) to provide 2.1 g of N-{3-[(4-chloro-2-ethoxymethyl-6,7-dimethyl-1H-imidazo[4,5-c]pyridin-1-yl)amino]propyl}methanesulfonamide as an amber oil.

### Part H

A pressure vessel was charged with N-{3-[(4-chloro-2-ethoxymethyl-6,7-dimethyl-1H-imidazo[4,5-c]pyridin-1-yl)amino]propyl}methanesulfonamide (0.10 g, 1 eq), benzylamine (0.40 mL, 12.7 eq), pyridine hydrochloride (0.15 g), and methanol (3 mL). The vessel was sealed and heated at 150° C. for 3 days. The reaction mixture was concentrated under reduced pressure. The residue was partitioned between water (25 mL) and dichloromethane (50 mL). The aqueous layer was adjusted to pH 14 and then extracted with dichloromethane (2×50 mL). The combined organics were concentrated under reduced pressure. The residue was purified by column chromatography (silica gel eluting with 5% methanol in chloroform) to provide 0.07 g of N-{3-[(4-benzylamino-2-ethoxymethyl-6,7-dimethyl-1H-imidazo[4,5-c]pyridin-1-yl)amino]propyl}methanesulfonamide as an amber oil. This procedure was repeated on a larger scale (1.71 g of N-{3-[(4-chloro-2-ethoxymethyl-6,7-dimethyl-1H-imidazo[4,5-c]pyridin-1-yl)amino]propyl)methanesulfonamide) to provide an additional 1.30 g of product.

### Part I

Under a nitrogen atmosphere, N-{3-[(4-benzylamino-2-ethoxymethyl-6,7-dimethyl-1H-imidazo[4,5-c]pyridin-1-yl)amino]propyl}methanesulfonamide (1.35 g, 1 eq) was combined with ammonium formate (1.94 g, 10.5 eq), methanol (60 mL) and ethanol (150 mL). The mixture was flushed with nitrogen for several minutes, 10% palladium on carbon (1.35 g) was added, and then the reaction mixture was heated to 80° C. Additional ammonium formate (2 g) was added every 2 hours for 8 hours and then the reaction mixture was allowed to cool to ambient temperature overnight. The reaction mixture was filtered through a layer of CELITE filter agent. The filter cake was washed with methanol and dichloromethane. The filtrate was concentrated under reduced pressure. The residue was partitioned between 10% sodium hydroxide (100 mL) and dichloromethane (100 mL). The aqueous layer was extracted with dichloromethane (3×100 mL). The aqueous layer was adjusted to pH 12 with hydrochloric acid followed by triethylamine and then extracted with dichloromethane (3×100 mL). The combined organics were concentrated under reduced pressure. The residue was purified by column chromatography (silica gel eluting with 10% methanol in chloroform) to provide 0.9 g of a white frothy solid. This material was recrystallized first from ethyl acetate and then from water and then dried under vacuum at 60° C. overnight to provide 0.48 g of N-{3-[(4-amino-2-ethoxymethyl-6,7-dimethyl-1H-imidazo[4,5-c]pyridin-1-yl)amino]propyl}methanesulfonamide as white fluffy needles, mp 147-149° C. ¹H NMR (300 MHz, DMSO-d₆) δ 6.99 (br t, J=5.8 Hz, 1H), 6.45 (br t, J=5.7 Hz, 1H), 5.77 (br s, 2H), 4.62 (s, 2H), 3.56 (q, J=7.0 Hz, 2H), 3.13-2.99 (m, 4H), 2.88 (s, 3H), 2.42 (s, 3H), 2.28 (s, 3H), 1.69 (pentet, J=7.0 Hz, 2H), 1.15 (t, J=7.0 Hz, 3H); MS (APCI) m/z 371 (M+H)⁺; Anal. Calcd for C₁₅H₂₆N₆O₃S: C, 48.63; H, 7.07; N, 22.68. Found: C, 48.72; H, 6.97; N, 22.66.

### Example 14

### N-{3-[(4-Amino-2-ethoxymethyl-6,7-dimethyl-1H-imidazo[4,5-c]pyridin-1-yl)amino]propyl}cyclohexanecarboxamide

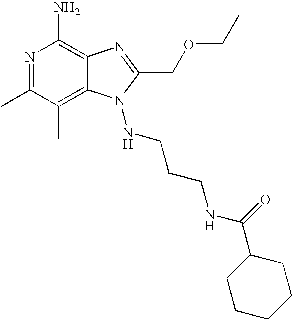

N-{3-[(4-Amino-2-ethoxymethyl-6,7-dimethyl-1H-imidazo[4,5-c]pyridin-1-yl)amino]propyl}cyclohexanecarboxamide was prepared according to the methods of Example 13 using cyclohexylcarbonyl chloride in lieu of methanesulfonyl chloride in Part G. The crude product was purified by column chromatography (silica gel eluting with 20% methanol in chloroform) to provide a clear oil. The oil was triturated with hot water to provide a solid. The solid was isolated by filtration and then dried under vacuum at 60° C. overnight to provide 0.31 g of N-{3-[(4-amino-2-ethoxymethyl-6,7-dimethyl-1H-imidazo[4,5-c]pyridin-1-yl)amino]propyl}cyclohexanecarboxamide as white needles, mp 166-168° C. ¹H NMR (300 MHz, DMSO-d₆) δ 7.66 (m, 1H), 6.41 (br t, J=5.5 Hz, 1H), 5.77 (br s, 2H), 4.62 (s, 2H), 3.56 (q, J=6.9 Hz, 2H), 3.20-2.90 (m, 4H), 2.40 (s, 3H), 2.28 (s, 3H), 2.04 (m, 1H), 1.76-1.48 (m, 6H), 1.39-1.04 (m, 6H), 1.14 (t, J=7.0 Hz, 3H); MS (APCI) m/z 403 (M+H)⁺; Anal. Calcd for C₂₁H₃₄N₆O₂: C, 62.66; H, 8.51; N, 20.88. Found: C, 62.59; H, 8.74; N, 21.03.

### Example 15

### N-{3-[(4-Amino-2,6,7-trimethyl-1H-imidazo[4,5-c]pyridin 1-yl)amino]propyl}methanesulfonamide

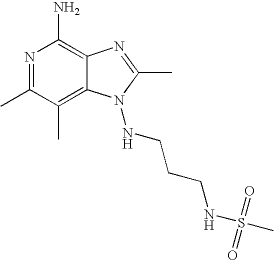

N-{3-[(4-Amino-2,6,7-trimethyl-1H-imidazo[4,5-c]pyridin-1-yl)amino]propyl}methanesulfonamide was prepared according to the methods of Example 13 using acetyl chloride in lieu of ethoxyacetyl chloride in Part B. The crude product was purified by column chromatography (silica gel eluting with a gradient of 20-40% methanol in chloroform) to provide 0.44 g of a white solid. This material was recrystallized first from isopropanol and then from water to provide 0.12 g of N-{3-[(4-amino-2,6,7-trimethyl-1H-imidazo[4,5-c]pyridin-1-yl)amino]propyl]methanesulfonamide as white needles, mp 215-217° C. ¹H NMR (300 MHz, DMSO-d₆) δ 7.01 (br t, J=5.6 Hz, 1H), 6.46 (br t, J=5.4 Hz, 1H), 5.60 (br s, 2H), 3.02 (m, 4H), 2.87 (s, 3H), 2.46 (s, 3H), 2.41 (s, 3H), 2.27 (s, 3H), 1.67 (pentet, J=7.1 Hz, 2H); MS (APCI) m/z 327 (M+H)⁺; Anal. Calcd for C₁₃H₂₂N₆O₂S.0.40H₂O: C, 46.80; H, 6.89; N, 25.19. Found: C, 46.74; H, 6.50; N, 25.13.

### Example 16

### 1-{3-[(4-Amino-2-ethoxymethyl-6,7-dimethyl-1H-imidazo[4,5-c]pyridin-1-yl)amino]propyl}-3-isopropylurea

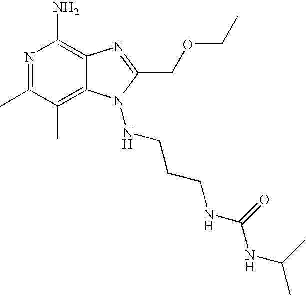

### Part A

Under a nitrogen atmosphere isopropyl isocyanate (0.73 mL, 1.05 eq) was added dropwise to a chilled (ice bath) solution of N¹-(4-chloro-2-ethoxymethyl-6,7-dimethyl-1H-imidazo[4,5-c]pyridin-1-yl)propane-1,3-diamine (2.2 g, 1 eq) in dichloromethane (50 mL). The reaction mixture was allowed to warm to ambient temperature and then it was concentrated under reduced pressure. The residue was recrystallized from acetonitrile to provide 1.81 g of 1-{3-[(4-chloro-2-ethoxymethyl-6,7-dimethyl-1H-imidazo[4,5-c]pyridin-1-yl)amino]propyl}-3-isopropylurea as white crystals.

### Part B

1-{3-[(4-Chloro-2-ethoxymethyl-6,7-dimethyl-1H-imidazo[4,5-c]pyridin-1-yl)amino]propyl}-3-isopropylurea (1.65 g) was treated with benzylamine using the method of Example 13 Part H. The crude product was purified by column chromatography (silica gel eluted with a gradient of 10-20% methanol in chloroform) to provide 0.90 g of an amber oil which was identified as N¹-(3-aminopropyl)-N⁴-(benzyl)-2-ethoxymethyl-6,7-dimethyl-1H-imidazo[4,5-c]pyridine-1,4-diamine.

### Part C

The material from Part B was treated with isopropyl isocyanate using the method of Part A. The crude product was purified by column chromatography (silica gel eluting with 7% methanol in chloroform) to provide 0.99 g of 1-{3-[(4-benzylamino-2-ethoxymethyl-6,7-dimethyl-1H-imidazo[4,5-c]pyridin-1-yl)amino]propyl}-3-isopropylurea as a pale oil.

### Part D

The benzyl group in the material from Part C was removed using the method of Example 13 Part I. The crude product was purified by column chromatography (silica gel eluting with 20% methanol in chloroform) to provide 0.22 g of 1-{3-[(4-amino-2-ethoxymethyl-6,7-dimethyl-1H-imidazo[4,5-c]pyridin-1-yl)amino]propyl}-3-isopropylurea as a white solid, mp 203-205° C. ¹H NMR (300 MHz, DMSO-d₆) δ 6.42 (br t, J=5.9 Hz, 1H), 5.86 (br s, 2H), 5.71 (br t, J=5.9 Hz, 1H), 5.61 (br d, J=7.6 Hz, 1H), 4.63 (s, 2H), 3.64 (m, 1H), 3.56 (q, J=7.0 Hz, 2H), 3.12-2.94 (m, 4H), 2.41 (s, 3H), 2.29 (s, 3H), 1.59 (pentet, J=6.9 Hz, 2H), 1.14 (t, J=7.0 Hz, 3H), 1.00 (d, J=6.6 Hz, 6H); MS (APCI) m/z 378 (M+H)⁺; Anal. Calcd for C₁₈H₃₁N₇O₂.0.25H₂O: C, 56.60; H, 8.31; N, 25.67. Found: C, 56.44; H, 8.59; N, 25.64.

### Example 17

### N¹-Cyclohexyl-2-ethoxymethyl-6,7-dimethyl-1H-imidazo[4,5-c]pyridine-1,4-diamine

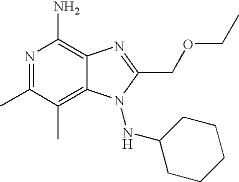

### Part A

Under a nitrogen atmosphere a mixture of 4-chloro-2-ethoxymethyl-6,7-dimethyl-1H-imidazo[4,5-c]pyridin-1-amine (5.0 g, 1 eq), cyclohexanone (4.1 mL g, 2.0 eq), pyridinium para-toluenesulfonate (5.0 g), and anhydrous acetonitrile (100 mL) was heated at reflux for 8 hours, allowed to cool to ambient temperature, and then concentrated under reduced pressure. The residue was partitioned between 10% sodium carbonate (100 mL) and dichloromethane (100 mL). The aqueous layer was separated and then extracted with dichloromethane (2×100 mL). The combined organics were concentrated under reduced pressure. The residue was purified by column chromatography (silica gel eluting with 3% methanol in dichloromethane) to provide 5.15 g of 4-chloro-N-cyclohexylidene-2-(ethoxymethyl)-6,7-dimethyl-1H-imidazo[4,5-c]pyridin-1-amine as a light yellow solid.

### Part B

The material from Part A was reduced with sodium borohydride according to the general method of Example 13 Part E to provide 4.77 g of 4-chloro-N-cyclohexyl-2-(ethoxymethyl)-6,7-dimethyl-1H-imidazo[4,5-c]pyridin-1-amine as a light tan solid.

### Part C

A pressure vessel was charged with the material from Part B (1 eq), benzylamine (22.2 mL, 13 eq), pyridine hydrochloride (13 g), and methanol (35 mL). The vessel was sealed and heated at 150° C. for 24 hours. The reaction mixture was allowed to cool to ambient temperature and then it was concentrated under reduced pressure. The residue was partitioned between 10% sodium carbonate (100 mL) and dichloromethane (100 mL). The aqueous layer was separated and then extracted with dichloromethane (2×100 mL). The combined organics were concentrated under reduced pressure. The residue was purified by column chromatography (silica gel eluting with 3% methanol in chloroform) to provide 3.89 g of N⁴-benzyl-N¹-cyclohexyl-2-(ethoxymethyl)-6,7-dimethyl-1H-imidazo[4,5-c]pyridine-1,4-diamine as an amber oil.

### Part D

The benzyl group was removed from the material from Part C using the general method of Example 13 Part I. The crude product was purified by column chromatography (silica gel eluting with 10% methanol in chloroform) to provide 1.5 g of a white solid. This material was recrystallized first from isopropanol and then from water to provide 1.00 g of N¹-cyclohexyl-2-ethoxymethyl-6,7-dimethyl-1H-imidazo[4,5-c]pyridine-1,4-diamine as white crystals, mp 108-110° C. ¹H NMR (300 MHz, DMSO-d₆) δ 6.48 (d, J=1.4 Hz, 1H), 5.76 (br s, 2H), 4.62 (s, 2H), 3.56 (q, J=7.0 Hz, 2H), 3.04 (m, 1H), 2.42 (s, 3H), 2.27 (s, 3H), 1.78-1.44 (m, 5H), 1.25-1.04 (m, 5H), 1.14 (t, J=7.0 Hz, 3H); MS (APCI) m/z 318 (M+H)⁺; Anal. Calcd for C₁₇H₂₇N₅O: C, 64.32; H, 8.57; N, 22.06. Found: C, 64.28; H, 8.40; N, 22.20.

### Example 18

### (4-Amino-1-cyclohexylamino-6,7-dimethyl-1H-imidazo[4,5-c]pyridin-2-yl)methanol

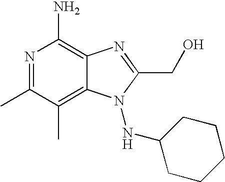

The ether group on N¹-cyclohexyl-2-ethoxymethyl-6,7-dimethyl-1H-imidazo[4,5-c]pyridine-1,4-diamine (1.0 g) was cleaved using the method of Example 12. The crude product was purified by column chromatography (silica gel eluting with a gradient of 20-40% methanol in chloroform) to provide 0.5 g of a white solid. This material was recrystallized from DMF to provide 0.25 g of a white crystalline solid. The solid was dissolved in methanol, concentrated under reduced pressure, and then dried under vacuum at 80° C. overnight to provide 0.19 g of (4-amino-1-cyclohexylamino-6,7-dimethyl-1H-imidazo[4,5-c]pyridin-2-yl)methanol as a white solid, mp>250° C. ¹H NMR (300 MHz, DMSO-d₆) δ 6.39 (d, J=1.7 Hz, 1H), 5.69 (br s, 2H), 5.40 (t, J=5.8 Hz, 2H), 4.66 (br d, J=5.3 Hz, 2H), 3.04 (m, 1H), 2.42 (s, 3H), 2.27 (s, 3H), 1.73-1.47 (m, 5H), 1.26-1.05 (m, 5H); MS (ESI) m/z 290 (M+H)⁺; Anal. Calcd for C₁₅H₂₃N₅O: C, 62.26; H, 8.01; N, 24.20. Found: C, 62.23; H, 8.34; N, 24.51.

### Example 19

### 2-Ethoxymethyl-6,7-dimethyl-N¹-(pyridin-3-yl)methyl-1H-imidazo[4,5-c]pyridine-1,4-diamine

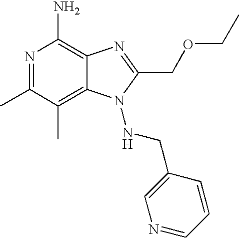

### Part A

Under a nitrogen atmosphere a mixture of 4-chloro-2-ethoxymethyl-6,7-dimethyl-1H-imidazo[4,5-c]pyridin-1-amine (0.50 g, 1 eq), 3-pyridine carboxaldehyde (0.37 mL, 2 eq), glacial acetic acid (1 mL), and acetonitrile (5 mL) was heated at reflux for 16 hours. The reaction mixture was allowed to cool to ambient temperature and then concentrated under reduced pressure. The residue was partitioned between 10% sodium carbonate (25 mL) and dichloromethane (25 mL). The aqueous layer was separated and then extracted with dichloromethane (2×25 mL). The combined organics were concentrated under reduced pressure to provide 0.67 g of 4-chloro-2-ethoxymethyl-6,7-dimethyl-N-(pyridin-3-yl)methylidene-1H-imidazo[4,5-c]pyridin-1-amine as an amber oil. The reaction was repeated on a larger scale (5.50 g of 4-chloro-2-ethoxymethyl-6,7-dimethyl-1H-imidazo[4,5-c]pyridin-1-amine) to provide 7.42 g of product as an amber oil.

### Part B

The material from Part A was reduced with sodium borohydride according to the method of Example 13 Part E. The crude product was purified by column chromatography (silica gel eluting with 5% methanol in chloroform) to provide an amber oil. The oil was triturated with ethyl acetate to provide 6.57 g of 4-chloro-2-ethoxymethyl-6,7-dimethyl-N-(pyridin-3-yl)methyl-1H-imidazo[4,5-c]pyridin-1-amine as light yellow crystals. A portion (0.50 g) of this material was recrystallized from isopropanol to provide 0.35 g of pure product as white needles, mp 143-145° C. ¹H NMR (300 MHz, CDCl₃) δ 8.69-8.61 (m, 2H), 7.57 (dt, J=7.8, 1.9 Hz, 1H), 7.31 (m, 1H), 5.86 (t, J=6.5 Hz, 1H), 4.76 (s, 2H), 4.30 (d, J=6.5 Hz, 2H), 3.62 (q, J=7.0 Hz, 2H), 2.67 (s, 3H), 2.57 (s, 3H), 1.22 (t, J=7.0 Hz, 3H); MS (APCI) m/z 346 (M+H)⁺; Anal. Calcd for C₁₇H₂₀ClN₅O: C, 59.04; H, 5.83; N, 20.25. Found: C, 59.04; H, 5.68; N, 20.21

### Part C

A pressure vessel was charged with 4-chloro-2-ethoxymethyl-6,7-dimethyl-N-(pyridin-3-yl)methyl-1H-imidazo[4,5-c]pyridin-1-amine (6.05 g, 1 eq), 4-methoxybenzylamine (23 mL, 10 eq), pyridine hydrochloride (10.1 g, 5 eq), and methanol (40 mL). The vessel was sealed and then heated in an oven at 150° C. for 48 hours. The reaction mixture was allowed to cool and then it was concentrated under reduced pressure. The residue was partitioned between 10% sodium carbonate (100 mL) and dichloromethane (100 mL). The aqueous layer was separated and then extracted with dichloromethane (2×100 mL). The combined organics were concentrated under reduced pressure. The residue was purified by column chromatography (silica gel eluting with a gradient of 0 to 10% methanol in ethyl acetate) to provide 4.9 g of 2-ethoxymethyl-N⁴-(4-methoxybenzyl)-6,7-dimethyl-N¹-(pyridin-3-yl)methyl-1H-imidazo[4,5-c]pyridine-1,4-diamine as an amber oil.

### Part D

The material from Part C was combined with trifluoroacetic acid (30 mL) and stirred under a nitrogen atmosphere for 48 hours. The reaction mixture was concentrated under reduced pressure. The residue was partitioned between 10% sodium hydroxide (100 mL) and dichloromethane (100 mL). The aqueous layer was separated and then extracted with dichloromethane (2×100 mL). The combined organics were concentrated under reduced pressure. The residue was purified by column chromatography (silica gel eluting with 10% methanol in chloroform) to provide about 3 g of an amber oil. This material was crystallized from acetonitrile to provide 2.2 g of tan crystals which were recrystallized from water to provide 1.89 g of 2-ethoxymethyl-6,7-dimethyl-N¹-(pyridin-3-yl)methyl-1H-imidazo[4,5-c]pyridine-1,4-diamine as white needles, mp 147-149° C. ¹H NMR (300 MHz, DMSO-d₆) δ 8.52 (dd, J=4.8, 1.6 Hz, 1H), 8.50 (d, J=1.9 Hz, 1H), 7.71 (dt, J=7.8, 1.9 Hz, 1H), 7.40 (dd, J=7.7, 4.8 Hz, 1H), 6.91 (t, J=5.6 Hz, 1H), 5.80 (br s, 2H), 4.53 (s, 2H), 4.23 (d, J=5.6 Hz, 2H), 3.55 (q, J=7.0 Hz, 2H), 2.46 (s, 3H), 2.29 (s, 3H), 1.13 (t, J=7.0 Hz, 3H); MS (ESI) m/z 327 (M+H)⁺; Anal. Calcd for C₁₇H₂₂N₆O: C, 62.56; H, 6.79; N, 25.75. Found: C, 62.52; H, 6.93; N, 26.09.

### Example 20

### [4-Amino-6,7-dimethyl-1-(pyridin-3-yl)methylamino-1H-imidazo[4,5-c]pyridin-2-yl]methanol

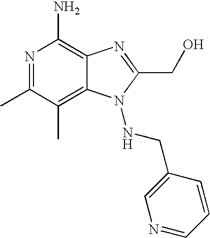

The ether group on 2-ethoxymethyl-6,7-dimethyl-N¹-(pyridin-3-yl)methyl-1H-imidazo[4,5-c]pyridine-1,4-diamine (1.70 g) was cleaved using the method of Example 12. The crude product was recrystallized from DMF to provide 0.6 g of light tan crystals. This material was dissolved in a mixture of methanol and dichloromethane, concentrated under reduced pressure, and then dried under vacuum at 80° C. overnight to provide 0.51 g of [4-amino-6,7-dimethyl-1-(pyridin-3-yl)methylamino-1H-imidazo[4,5-c]pyridin-2-yl]methanol as a light tan solid, mp 258-260° C. ¹H NMR (300 MHz, DMSO-d₆) δ 8.56-8.49 (m, 2H), 7.73 (dt, J=7.8, 1.8 Hz, 1H), 7.39 (dd, J=7.7, 4.9 Hz, 1H), 6.83 (t, J=5.7 Hz, 1H), 5.73 (br s, 2H), 5.51 (t, J=5.7 Hz, 1H), 4.57 (d, J=5.7 Hz, 2H), 4.26 (d, J=5.7 Hz, 2H), 2.45 (s, 3H), 2.28 (s, 3H); MS (APCI) m/z 299 (M+H)⁺; Anal. Calcd for C₁₅H₁₈N₆O: C, 60.39; H, 6.08; N, 28.17. Found: C, 60.29; H, 5.96; N, 28.09.

### Example 21

### N¹-Benzyl-2-ethoxymethyl-6,7-dimethyl-1H-imidazo[4,5-c]pyridine-1,4-diamine

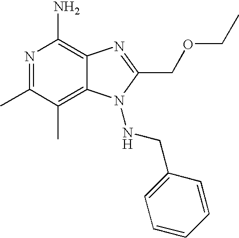

### Part A

A 5 mL process vial was charged with N¹-benzyl-4-chloro-2-ethoxymethyl-6,7-dimethyl-1H-imidazo[4,5-c]pyridin-1-amine (0.25 g, 1 eq; prepared according to the general methods of Example 19 Parts A and B using benzaldehyde in lieu of 3-pyridine carboxaldehyde in Part A), 4-methoxybenzylamine (0.95 mL, 10 eq), pyridine hydrochloride (0.42 g, 5 eq), and 2,2,2-trifluoroethanol (2.5 mL). The vessel was heated at 160° C. in a microwave for 2 hours. The reaction was rerun on a larger scale (2.81 g of 3-pyridine carboxaldehyde) using 3 separate 20 mL process vials. The reaction mixtures were combined and concentrated under reduced pressure. The residue was partitioned between 10% sodium carbonate (100 mL) and dichloromethane (100 mL). The aqueous layer was separated and then extracted with dichloromethane (2×100 mL). The combined organics were concentrated under reduced pressure. The residue was purified by column chromatography (silica gel eluting with 4% methanol in chloroform) to provide 3.9 g of a brown oil. This material was purified by column chromatography (silica gel eluting with 20% hexanes in ethyl acetate) to provide 3.0 g of N¹-benzyl-2-ethoxymethyl-N⁴-(4-methoxybenzyl)-6,7-dimethyl-1H-imidazo[4,5-c]pyridine-1,4-diamine as a light brown oil.

### Part B

The material from Part A was combined with trifluoroacetic acid (30 mL) and stirred under a nitrogen atmosphere for 16 hours. The reaction mixture was concentrated under reduced pressure. The residue was partitioned between 10% sodium hydroxide (100 mL) and dichloromethane (100 mL). The aqueous layer was separated and then extracted with dichloromethane (2×100 mL). The combined organics were concentrated under reduced pressure. The residue was purified by column chromatography (silica gel eluting with 10% methanol in chloroform) to provide about a clear oil which slowly crystallized. This material was recrystallized from acetonitrile to provide 1.54 g of N¹-benzyl-2-ethoxymethyl-6,7-dimethyl-1H-imidazo[4,5-c]pyridine-1,4-diamine as white needles, mp 149-151° C. ¹H NMR (300 MHz, DMSO-d₆) δ 7.43-7.27 (m, 5H), 6.78 (t, J=5.8 Hz, 1H), 5.79 (br s, 2H), 4.49 (s, 2H), 4.18 (d, J=5.8 Hz, 2H), 3.54 (q, J=7.0 Hz, 2H), 2.49 (s, 3H), 2.30 (s, 3H), 1.13 (t, J=7.0 Hz, 3H); MS (ESI) m/z 326 (M+H)⁺; Anal. Calcd for C₁₈H₂₃N₅O: C, 66.44; H, 7.12; N, 21.52. Found: C, 66.48; H, 7.42; N, 21.79.

### Example 22

### (4-Amino-1-benzylamino-6,7-dimethyl-1H-imidazo[4,5-c]pyridin-2-yl)methanol

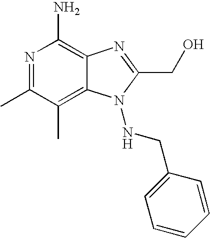

The ether group on N¹-benzyl-2-ethoxymethyl-6,7-dimethyl-1H-imidazo[4,5-c]pyridine-1,4-diamine (0.87 g) was cleaved using the method of Example 12. The crude product was purified by column chromatography (silica gel eluting with a gradient of 20-40% methanol in chloroform) to provide 0.57 g of a white solid. This material was recrystallized from DMF to provide 0.45 g of a white crystalline solid. The solid was dissolved in a mixture of methanol and dichloromethane, concentrated under reduced pressure, and then dried under vacuum at 80° C. overnight to provide 0.35 g of (4-amino-1-benzylamino-6,7-dimethyl-1H-imidazo[4,5-c]pyridin-2-yl)methanol as a white solid, mp>250° C. ¹H NMR (300 MHz, DMSO-d₆) δ 7.45-7.25 (m, 5H), 6.70 (t, J=5.9 Hz, 1H), 5.74 (br s, 2H), 5.46 (br t, J=5.7 Hz, 1H), 4.54 (d, J=5.4 Hz, 2H), 4.20 (d, J=5.9 Hz, 2H), 2.49 (s, 3H), 2.29 (s, 3H); MS (ESI) m/z 298 (M+H)⁺; Anal. Calcd for C₁₆H₁₉N₅O: C, 64.63; H, 6.44; N, 23.55. Found: C, 64.38; H, 6.36; N, 23.63.

### Example 23

### N¹-Cyclobutyl-2-ethoxymethyl-6,7-dimethyl-1H-imidazo[4,5-c]pyridine-1,4-diamine

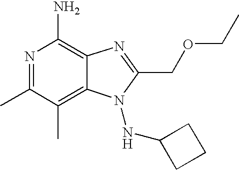

N¹-Cyclobutyl-2-ethoxymethyl-6,7-dimethyl-1H-imidazo[4,5-c]pyridine-1,4-diamine was prepared according to the general methods of Example 21 using cyclobutanone in lieu of benzaldehyde. The crude product was purified by column chromatography (silica gel eluting with 10% methanol in chloroform) to provide 0.6 g of a white solid. This material was recrystallized first from acetonitrile and then from water and then dried under vacuum at 70° C. overnight to provide 0.41 g of N¹-cyclobutyl-2-ethoxymethyl-6,7-dimethyl-1H-imidazo[4,5-c]pyridine-1,4-diamine as white needles, mp 139-141° C. ¹H NMR (300 MHz, DMSO-d₆) δ 6.62 (d, J=3.8 Hz, 1H), 5.77 (br s, 2H), 4.61 (s, 2H), 3.72 (m, 1H), 3.57 (q, J=7.0 Hz, 2H), 2.41 (s, 3H), 2.28 (s, 3H), 2.04 (m, 2H), 1.86 (m, 2H), 1.75-1.48 (m, 2H), 1.14 (t, J=7.0 Hz, 3H); MS (APCI) m/z 290 (M+H)⁺; Anal. Calcd for C₁₅H₂₃N₅O: C, 62.26; H, 8.01; N, 24.20. Found: C, 62.28; H, 7.97; N, 24.51.

### Example 24

### (4-Amino-1-cyclobutylamino-6,7-dimethyl-1H-imidazo[4,5-c]pyridin-2-yl)methanol

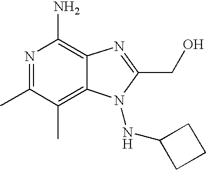

The ether group on N¹-cyclobutyl-2-ethoxymethyl-6,7-dimethyl-1H-imidazo[4,5-c]pyridine-1,4-diamine (1.24 g) was cleaved using the method of Example 12. The crude product was recrystallized first from DMF and then from water and then dried under vacuum at 80° C. overnight to provide 0.25 g of (4-amino-1-cyclobutylamino-6,7-dimethyl-1H-imidazo[4,5-c]pyridin-2-yl)methanol as white crystals, mp 211-213° C. ¹H NMR (300 MHz, DMSO-d₆) δ 6.55 (d, J=4.2 Hz, 1H), 5.70 (br s, 2H), 5.45 (t, J=5.8 Hz, 1H), 4.64 (d, J=5.7 Hz, 2H), 3.74 (pentet, J=7.5, 4.2 Hz, 1H), 2.42 (s, 3H), 2.27 (s, 3H), 2.05 (m, 2H), 1.86 (m, 2H), 1.74-1.48 (m, 2H); MS (ESI) m/z 262 (M+H)⁺; Anal. Calcd for C₁₃H₁₉N₅O.0.25H₂O: C, 58.74; H, 7.39; N, 26.34. Found: C, 58.65; H, 7.31; N, 26.51.

### Example 25

### 2-Ethoxymethyl-6,7-dimethyl-N¹-(tetrahydropyran-4-yl)-1H-imidazo[4,5-c]pyridine-1,4-diamine

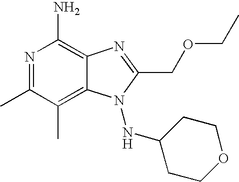

### Part A

Under a nitrogen atmosphere, a mixture of 4-chloro-2-ethoxymethyl-6,7-dimethyl-1H-imidazo[4,5-c]pyridin-1-amine (10.0 g, 1 eq), tetrahydro-4H-pyran-4-one (6.2 mL, 1.5 eq), glacial acetic acid (20 mL), and acetonitrile (100 mL) was heated at reflux for 16 hours. The reaction mixture was concentrated under reduced pressure. The residue was partitioned between 10% sodium carbonate (100 mL) and dichloromethane (100 mL). A tan precipitate was isolated by filtration, rinsed with dichloromethane and water, and dried to provide 9.9 g of 4-chloro-2-ethoxymethyl-6,7-dimethyl-N-(tetrahydropyran-4-ylidene)-1H-imidazo[4,5-c]pyridin-1-amine as a tan solid. A portion (100 mg) of this material was recrystallized from ethyl acetate to provide 60 mg of pure product as white crystals, mp 143-145° C. ¹H NMR (300 MHz, CDCl₃) δ 4.60 (s, 2H), 4.03 (m, 2H), 3.76 (m, 2H), 3.62 (q, J=7.0 Hz, 2H), 2.84 (m, 2H), 2.56 (s, 3H), 2.35 (s, 3H), 2.24 (m, 2H), 1.21 (t, J=7.0 Hz, 3H); MS (ESI) m/z 337 (M+H)⁺; Anal. Calcd for C₁₆H₂₁ClN₄O₂: C, 57.06; H, 6.28; N, 16.63. Found: C, 56.84; H, 6.39; N, 16.62.

### Part B

Under a nitrogen atmosphere sodium borohydride (3.3 g, 3 eq) was added in portions over a period of 5 minutes to a solution of material from Part A (9.8 g) in methanol (200 mL). After 2 hours saturated ammonium chloride (50 mL) was added and the reaction mixture was stirred for 5 minutes. The methanol was removed under reduced pressure. Sodium carbonate (5 g) was added to the aqueous residue. A precipitate was isolated by filtration and rinsed with water. The filtrate was extracted with dichloromethane (3×100 mL). The combined organics were concentrated under reduced pressure to provide an amber oil. The oil and the isolated solid were combined and purified by column chromatography (silica gel eluting with 3% methanol in chloroform) to provide 7.95 g of a clear oil which slowly solidified. A portion (1.0 g) was recrystallized from isopropanol to provide 0.6 g of 4-chloro-2-ethoxymethyl-6,7-dimethyl-N-(tetrahydropyran-4-yl)-1H-imidazo[4,5-c]pyridin-1-amine as white crystals, mp 137-139° C. ¹H NMR (300 MHz, CDCl₃) δ 5.58 (d, J=3.5 Hz, 1H), 4.92 (br s, 2H), 4.00 (m, 2H), 3.63 (q, J=7.0 Hz, 2H), 3.45-3.25 (m, 3H), 2.66 (s, 3H), 2.56 (s, 3H), 1.83-1.40 (m, 4H), 1.25 (t, J=7.0 Hz, 3H); MS (APCI) m/z 339 (M+H)⁺; Anal. Calcd for C₁₆H₂₃ClN₄O₂: C, 56.72; H, 6.84; N, 16.53. Found: C, 56.70; H, 6.71; N, 16.64.

### Part C

4-Chloro-2-ethoxymethyl-6,7-dimethyl-N-(tetrahydropyran-4-yl)-1H-imidazo[4,5-c]pyridin-1-amine (4.9 g) was treated with 4-methoxybenzylamine using the method of Example 19 Part C. The crude product was purified by column chromatography (silica gel eluting with 2% methanol in chloroform) to provide 4.35 g of an amber oil. The oil was purified by column chromatography (silica gel eluting with ethyl acetate) to provide 3.3 g of 2-ethoxymethyl-N⁴-(4-methoxybenzyl)-6,7-dimethyl-N¹-(tetrahydropyran-4-yl)-1H-imidazo[4,5-c]pyridin-1-amine a light amber oil.

### Part D

A mixture of the material from Part C and trifluoroacetic acid (20 mL) was stirred for 4 days under a nitrogen atmosphere and then concentrated under reduced pressure. The residue was partitioned between 10% sodium hydroxide (100 mL) and dichloromethane (100 mL). The aqueous layer was separated and then extracted with dichloromethane (100 mL). The combined organics were concentrated under reduced pressure. The residue was purified by column chromatography (silica gel eluting with a gradient of 5-20% methanol in chloroform) to provide about 1.8 g of an amber solid. This material was recrystallized from water to provide 1.15 g of 2-ethoxymethyl-6,7-dimethyl-N¹-(tetrahydropyran-4-yl)-1H-imidazo[4,5-c]pyridine-1,4-diamine as white crystals, mp 120-122° C. ¹H NMR (300 MHz, DMSO-d₆) δ 6.65 (d, J=1.6 Hz, 1H), 5.78 (br s, 2H), 4.64 (s, 2H), 3.83 (m, 2H), 3.57 (q, J=7.0 Hz, 2H), 3.40-3.15 (m, 3H), 2.44 (s, 3H), 2.28 (s, 3H), 1.54-1.32 (m, 4H), 1.14 (t, J=7.0 Hz, 3H); MS (APCI) m/z 320 (M+H)⁺; Anal. Calcd for C₁₆H₂₅N₅O₂: C, 60.17; H, 7.89; N, 21.93. Found: C, 59.95; H, 7.86; N, 21.83.

### Example 26

### [4-Amino-6,7-dimethyl-1-(tetrahydropyran-4-yl)amino-1H-imidazo[4,5-c]pyridin-2-yl]methanol

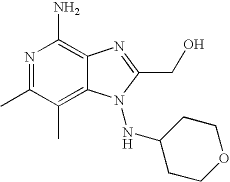

Under a nitrogen atmosphere boron tribromide (11.0 mL of 1 M in dichloromethane, 2.5 eq) was added dropwise to a chilled (ice bath) solution of 2-ethoxymethyl-6,7-dimethyl-N¹-(tetrahydropyran-4-yl)-1H-imidazo[4,5-c]pyridine-1,4-diamine (1.40 g, 1 eq) in dichloromethane (30 mL). The reaction was kept cool for 2 hours and then allowed to stir at ambient temperature over the weekend. Analysis by HPLC indicated that the tetrahydropyran ring had opened to give 3-{[4-amino-2-(hydroxymethyl)-6,7-dimethyl-1H-imidazo[4,5-c]pyridin-1-yl]amino}-5-bromopentan-1-ol. The reaction mixture was quenched with methanol (20 mL) and stirred for 5 minutes. Hydrochloric acid (30 mL of 6 N) was added and the reaction mixture was stirred overnight. The reaction mixture was adjusted to pH 13 with sodium hydroxide and then it was extracted with dichloromethane (3×100 mL). The combined organics were concentrated under reduced pressure to provide about 1.2 g of a brown oil. The oil was dissolved in methanol, triethylamine was added, and the mixture was heated to reflux. After 24 hours analysis indicated that the cyclization was stalled at about 60% completion. The reaction mixture was concentrated under reduced pressure. The residue was combined with ethanol (50 mL) and sodium ethoxide (5 mL) and heated to reflux. Analysis after 16 hours indicated that the cyclization had not progressed. The reaction mixture was concentrated under reduced pressure. The residue was partitioned between 10% sodium carbonate (100 mL) and dichloromethane (100 mL). The aqueous layer was separated and then extracted with dichloromethane (2×100 ml). The combined organics were concentrated under reduced pressure. The residue was purified by HPFC (silica gel eluted with a gradient of 15-30% methanol in dichloromethane) to provide 0.25 g of a white solid. This solid was recrystallized first from a mixture of methanol and DMF and then from DMF. The resulting crystalline solid was dissolved in water and the solution was concentrated under reduced pressure to provide 90 mg of a white solid. The solid was dried under vacuum at 80° C. for 16 hours to provide 90 mg of [4-amino-6,7-dimethyl-1-(tetrahydropyran-4-yl)amino-1H-imidazo[4,5-c]pyridin-2-yl]methanol as white crystals, mp>250° C. ¹H NMR (300 MHz, DMSO-d₆) δ 6.56 (d, J=2.1 Hz, 1H), 5.71 (br s, 2H), 5.44 (t, J=5.8 Hz, 1H), 4.67 (d, J=5.6 Hz, 2H), 3.89-3.74 (m, 2H), 3.43-3.15 (m, 3H), 2.44 (s, 3H), 2.28 (s, 3H), 1.59-1.32 (m, 4H); MS (ESI) m/z 292 (M+H)⁺; Anal. Calcd for C₁₄H₂₁N₅O₂: C, 57.72; H, 7.27; N, 24.04. Found: C, 57.67; H, 7.26; N, 24.29.

### Example 27

### 2-Ethoxymethyl-6,7-dimethyl-N-(tetrahydropyranyl)-1H-imidazo[4,5-c]pyridin-1-amine

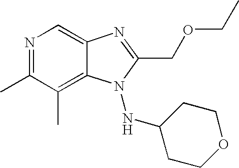

Under a nitrogen atmosphere, 4-chloro-2-ethoxymethyl-6,7-dimethyl-N-(tetrahydropyran-4-yl)-1H-imidazo[4,5-c]pyridin-1-amine (1.00 g, 1 eq) was combined with ammonium formate (1.94 g, 10.5 eq), methanol (40 mL) and ethanol (80 mL). The mixture was flushed with nitrogen for several minutes, 10% palladium on carbon (1.00 g) was added, and then the reaction mixture was heated to 80° C. for 3 hours. The reaction mixture was allowed to cool to ambient temperature and then it was filtered through a layer of CELITE filter agent. The filtrate was concentrated under reduced pressure. The residue was partitioned between 5% sodium hydroxide (100 mL) and dichloromethane (100 mL). The aqueous layer was extracted with dichloromethane (2×100 mL). The combined organics were dried over sodium sulfate, filtered, and then concentrated under reduced pressure. The residue was purified by column chromatography (silica gel eluting with 3% methanol in chloroform) to provide 0.52 g of a clear oil which slowly solidified. This material was dried under vacuum at 40° C. for 16 hours to provide 0.52 g of 2-ethoxymethyl-6,7-dimethyl-N-(tetrahydropyran-4-yl)-1H-imidazo[4,5-c]pyridin-1-amine as a white solid, mp 94-97° C. ¹H NMR (300 MHz, CDCl₃) δ 8.75 (s, 1H), 5.46 (d, J=3.2 Hz, 1H), 4.87 (br s, 2H), 4.00 (m, 2H), 3.63 (q, J=7.0 Hz, 2H), 3.45-3.25 (m, 3H), 2.68 (s, 3H), 2.60 (s, 3H), 1.77-1.44 (m, 4H), 1.26 (t, J=7.0 Hz, 3H); MS (APCI) m/z 305 (M+H)⁺; Anal. Calcd for C₁₆H₂₄N₄O₂.0.50H₂O: C, 61.32; H, 8.04; N, 17.88. Found: C, 60.92; H, 7.93; N, 17.75.

### Example 28

### N¹-Cyclohexyl-2,6,7-trimethyl-1H-imidazo[4,5-c]pyridine-1,4-diamine

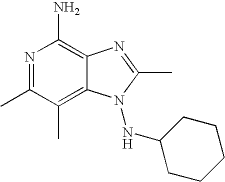

### Part A

A mixture of tert-butyl 2-(3-amino-2-chloro-5,6-dimethylpyridin-4-yl)hydrazinecarboxylate (10 g, 1 eq), triethyl orthoacetate (8.31 mL, 1.3 eq), pyridine hydrochloride (5.0 g), and toluene (210 mL) was heated at reflux under a nitrogen atmosphere for 2 hours and then concentrated under reduced pressure. The residue was partitioned between 10% sodium carbonate (100 mL) and dichloromethane (100 mL). The aqueous layer was separated and then extracted with dichloromethane (2×100 ml). The combined organics were concentrated under reduced pressure to provide 8.57 g of tert-butyl 4-chloro-2,6,7-trimethyl-1H-imidazo[4,5-c]pyridin-1-ylcarbamate as a brown frothy solid.

### Part B

Under a nitrogen atmosphere trifluoroacetic acid (30 mL) was slowly added to a chilled (ice bath) solution of the material from Part A in dichloromethane (100 mL). The reaction mixture was kept cool for 1 hour, then allowed to warm to ambient temperature overnight and then concentrated under reduced pressure. The residue was dissolved in water and the pH of the solution was adjusted to 14 with 50% sodium hydroxide. The mixture was adjusted to pH 12 with hydrochloric acid and sodium carbonate and then it was extracted with dichloromethane (10×100 mL). The combined organics were concentrated under reduced pressure. The residue was purified by HPFC (silica gel eluted with a gradient of 5-20% methanol in dichloromethane) to provide 4.0 g of 4-chloro-2,6,7-trimethyl-1H-imidazo[4,5-c]pyridin-1-amine as a light tan solid.

### Part C

A mixture of 4-chloro-2,6,7-trimethyl-1H-imidazo[4,5-c]pyridin-1-amine (2.0 g, 1 eq), cyclohexanone (1.97 mL, 2 eq), glacial acetic acid (5.0 mL), and anhydrous acetonitrile (20 mL) was heated at reflux under a nitrogen atmosphere for 24 hours. The reaction mixture was allowed to cool to ambient temperature and then it was concentrated under reduced pressure. The residue was partitioned between 10% sodium carbonate (100 mL) and dichloromethane (100 mL). The aqueous layer was separated and then extracted with dichloromethane (2×100 ml). The combined organics were concentrated under reduced pressure to provide 2.90 g of 4-chloro-N-cyclohexylidene-2,6,7-trimethyl-1H-imidazo[4,5-c]pyridin-1-amine as an amber oil.

### Part D

The material from Part C was reduced with sodium borohydride using the method of Example 25 Part B. The crude product was purified by HPFC (silica gel eluted with a gradient of 0-11% methanol in dichloromethane) to provide 2.20 g of 4-chloro-N-cyclohexyl-2,6,7-trimethyl-1H-imidazo[4,5-c]pyridin-1-amine as a white solid.

### Part E

4-Chloro-N-cyclohexyl-2,6,7-trimethyl-1H-imidazo[4,5-c]pyridin-1-amine (2.05 g) was treated with 4-methoxybenzylamine using the method of Example 21 Part A. The crude product was purified by HPFC (silica gel eluted with a gradient of 2-11% methanol in ethyl acetate) to provide about 2.7 g of N¹-cyclohexyl-N⁴-(4-methoxybenzyl)-2,6,7-trimethyl-1H-imidazo[4,5-c]pyridine-1,4-diamine as a light amber oil.

### Part F

A mixture of the material from Part E and trifluoroacetic acid (30 mL) was stirred for 2 days under a nitrogen atmosphere and then concentrated under reduced pressure. The residue was diluted with water (100 mL) and dichloromethane (100 mL) and the pH of the aqueous layer was adjusted to 13 with sodium hydroxide. The aqueous layer was separated and then extracted with dichloromethane (2×100 mL). The combined organics were concentrated under reduced pressure. The residue was purified by HPFC (silica gel eluted with a gradient of 5-25% methanol in dichloromethane) to provide 1.34 g of a white solid. This material was recrystallized from acetonitrile and then dried under vacuum at 80° C. for 16 hours to provide 1.10 g of N¹-cyclohexyl-2,6,7-trimethyl-1H-imidazo[4,5-c]pyridine-1,4-diamine as white crystals, mp 242-244° C. ¹H NMR (300 MHz, DMSO-d₆) δ 6.46 (d, J=1.1 Hz, 1H), 5.62 (br s, 2H), 2.94 (m, 1H), 2.46 (s, 3H), 2.40 (s, 3H), 2.26 (s, 3H), 1.78-1.44 (m, 5H), 1.16 (m, 5H); MS (ESI) m/z 274 (M+H)⁺; Anal. Calcd for C₁₅H₂₃N₅: C, 65.90; H, 8.48; N, 25.62. Found: C, 65.71; H, 8.55; N, 25.64.

### Example 29

### N¹-Cyclohexyl-2-ethyl-6,7-dimethyl-1H-imidazo[4,5-c]pyridine-1,4-diamine

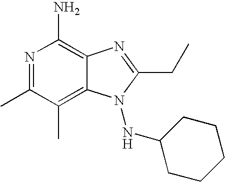

N¹-Cyclohexyl-2-ethyl-6,7-dimethyl-1H-imidazo[4,5-c]pyridine-1,4-diamine was prepared according to the methods of Example 28 using triethyl orthopropionate in lieu of triethyl orthoacetate in Part A. The crude product was purified by HPFC (silica gel eluted with a gradient of 7-25% methanol in dichloromethane) to provide 1.63 g of a white solid. This material was recrystallized from acetonitrile to provide 0.96 g of N-cyclohexyl-2-ethyl-6,7-dimethyl-1H-imidazo[4,5-c]pyridine-1,4-diamine as white crystals, mp 190-192° C. ¹H NMR (300 MHz, DMSO-d₆) δ 6.44 (d, J=1.1 Hz, 1H), 5.58 (br s, 2H), 3.04-2.66 (m, 3H), 2.40 (s, 3H), 2.27 (s, 3H), 1.75-1.41 (m, 5H), 1.29 (t, J=7.5 Hz, 3H), 1.24-1.01 (m, 5H); MS (ESI) m/z 288 (M+H)⁺; Anal. Calcd for C₁₆H₂₅N₅: C, 66.87; H, 8.77; N, 24.37. Found: C, 66.70; H, 8.47; N, 24.40.

### Example 30

### N¹-Cyclohexyl-6,7-dimethyl-1H-imidazo[4,5-c]pyridine-1,4-diamine

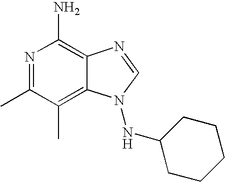

N¹-Cyclohexyl-6,7-dimethyl-1H-imidazo[4,5-c]pyridine-1,4-diamine was prepared according to the methods of Example 28 using triethyl orthoformate in lieu of triethyl orthoacetate in Part A. The crude product was recrystallized from a mixture of methanol and DMF to provide N¹-cyclohexyl-6,7-dimethyl-1H-imidazo[4,5-c]pyridine-1,4-diamine as white crystals, mp 259-261° C. ¹H NMR (300 MHz, DMSO-d₆) δ 7.92 (s, 1H), 6.57 (d, J=3.3 Hz, 1H), 5.73 (br s, 2H), 3.00 (m, 1H), 2.43 (s, 3H), 2.27 (s, 3H), 1.90-1.44 (m, 5H), 1.36-1.04 (m, 5H); MS (ESI) m/z 260 (M+H)⁺; Anal. Calcd for C₁₄H₂₁N₅: C, 64.84; H, 8.16; N, 27.00. Found: C, 64.77; H, 8.26; N, 27.04.

### Example 31

### 2-Butyl-N¹-cyclohexyl-6,7-dimethyl-1H-imidazo[4,5-c]pyridine-1,4-diamine

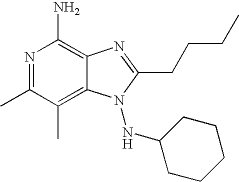

2-Butyl-N¹-cyclohexyl-6,7-dimethyl-1H-imidazo[4,5-c]pyridine-1,4-diamine was prepared according to the methods of Example 28 using triethyl orthovalerate in lieu of triethyl orthoacetate in Part A. The crude product was purified by HPFC (silica gel eluted with a gradient of 7-20% methanol in dichloromethane) to provide 1.42 g of a white solid. This material was recrystallized from ethyl acetate and then dried in a vacuum oven at 60° C. for 2 days to provide 1.25 g of 2-butyl-N¹-cyclohexyl-6,7-dimethyl-1H-imidazo[4,5-c]pyridine-1,4-diamine as white crystals, mp 160-162° C. ¹H NMR (300 MHz, DMSO-d₆) δ 6.44 (m, 1H), 5.57 (br s, 2H), 3.04-2.62 (m, 3H), 2.39 (s, 3H), 2.27 (s, 3H), 1.83-1.01 (m, 14H), 0.93 (t, J=7.3 Hz, 3H); MS (ESI) m/z 316 (M+H)⁺; Anal. Calcd for C₁₈H₂₉N₅: C, 68.53; H, 9.27; N, 22.20. Found: C, 68.46; H, 9.35; N, 22.17.

### Example 32

### 2,6,7-Trimethyl-N¹-(tetrahydropyran-4-yl)-1H-imidazo[4,5-c]pyridine-1,4-diamine

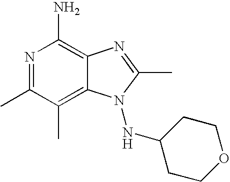

2,6,7-Trimethyl-N¹-(tetrahydropyran-4-yl)-1H-imidazo[4,5-c]pyridine-1,4-diamine was prepared according to the methods of Example 28 using tetrahydro-4H-pyran-4-one in lieu of cyclohexanone in Part C. The crude product was purified by HPFC (silica gel eluted with a gradient of 10-30% methanol in dichloromethane) to provide 1.29 g of a white solid. This material was recrystallized from water and then dried in a vacuum oven at 80° C. for 16 hours to provide 1.17 g of 2,6,7-trimethyl-N¹-(tetrahydropyran-4-yl)-1H-imidazo[4,5-c]pyridine-1,4-diamine as white crystals, mp 240-242° C. ¹H NMR (300 MHz, DMSO-d₆) δ 6.63 (d, J=1.6 Hz, 1H), 5.62 (br s, 2H), 3.82 (m, 2H), 3.35-3.13 (m, 3H), 2.47 (s, 3H), 2.42 (s, 3H), 2.27 (s, 3H), 1.57-1.30 (m, 4H); MS (ESI) m/z 276 (M+H)⁺; Anal. Calcd for C₁₄H₂₁N₅O: C, 61.07; H, 7.69; N, 25.43. Found: C, 60.87; H, 7.61; N, 25.51.

### Example 33

### N¹-Cyclohexyl-6,7-dimethyl-2-propyl-1H-imidazo[4,5-c]pyridine-1,4-diamine

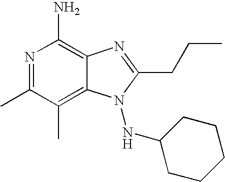

### Part A

Butyryl chloride (2.0 mL g, 1.1 eq) was added dropwise to a chilled (ice bath) solution of tert-butyl 2-(3-amino-2-chloro-5,6-dimethylpyridin-4-yl)hydrazinecarboxylate (5.0 g, 1 eq) and triethylamine (3.6 mL, 1.5 eq) in dichloromethane (150 mL). The reaction mixture was allowed to warm to ambient temperature. After 4 hours additional butyryl chloride (0.25 eq) was added. After 2 hours the reaction mixture was washed with water (150 mL) and then concentrated under reduced pressure. The residue was dissolved in a mixture of ethanol (100 mL) and water (20 mL). Sodium hydroxide (2.1 g, 3 eq) was added and the reaction mixture was stirred at ambient temperature. After 2 hours the reaction mixture was concentrated under reduced pressure. The residue was partitioned between water (100 mL) and dichloromethane (100 mL). The aqueous layer was separated and then extracted with dichloromethane (2×100 mL). The combined organics were filtered and then concentrated under reduced pressure to provide 5.21 g of tert-butyl 4-chloro-6,7-dimethyl-2-propyl-1H-imidazo[4,5-c]pyridin-1-ylcarbamate as an orange foamy solid.

### Part B

N¹-Cyclohexyl-6,7-dimethyl-2-propyl-1H-imidazo[4,5-c]pyridine-1,4-diamine was prepared according to the methods of Example 28 Parts B through P using tert-butyl 4-chloro-6,7-dimethyl-2-propyl-1H-imidazo[4,5-c]pyridin-1-ylcarbamate in lieu of tert-butyl 4-chloro-2,6,7-trimethyl-1H-imidazo[4,5-c]pyridin-1-ylcarbamate in Part B. The crude product was purified by HPFC (silica gel eluted with a gradient of 7-25% methanol in dichloromethane) to provide 1.42 g of a white solid. This material was recrystallized from isopropanol and then dried in a vacuum oven at 60° C. for 3 days to provide 1.06 g of N¹-cyclohexyl-6,7-dimethyl-2-propyl-1H-imidazo[4,5-c]pyridine-1,4-diamine as white crystals, mp 177-179° C. ¹H NMR (300 MHz, DMSO-d₆) δ 6.44 (d, J=1.0 Hz, 1H), 5.58 (br s, 2H), 3.05-2.59 (m, 3H), 2.40 (s, 3H), 2.27 (s, 3H), 1.78 (sextet, J=7.5 Hz, 2H), 1.71-1.40 (m, 5H), 1.29-1.04 (m, 5H), 0.97 (t, J=7.4 Hz, 3H); MS (ESI) m/z 302 (M+H)⁺; Anal. Calcd for C₁₇H₂₇N₅: C, 67.74; H, 9.03; N, 23.23. Found: C, 67.57; H, 9.03; N, 23.34.

### Example 34

### N¹-Benzyl-2-ethxoymethyl-1H-imidazo[4,5-c][1,5]naphthyridine-1,4-diamine

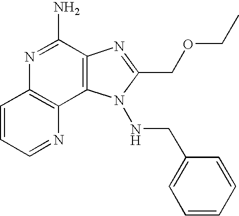

### Part A

Under a nitrogen atmosphere glacial acetic acid (3 mL) was added to a suspension of 2-ethxoymethyl-1H-imidazo[4,5-c][1,5]naphthyridin-1-amine (0.75 g, 1 eq) in acetonitrile (30 mL) and a solution was obtained. Benzaldehyde (0.35 mL, 1.1 eq) was added and the reaction mixture was heated to 100° C. After 14 hours additional benzaldehyde (0.5 mL) was added and the reaction mixture was heated for an additional 3 hours. The reaction mixture was allowed to cool to ambient temperature and then it was concentrated under reduced pressure to provide a brown oil. The oil was dissolved in chloroform (45 mL), washed sequentially with 10% sodium carbonate (2×15 mL) and brine (15 mL), dried over sodium sulfate, filtered, and then concentrated under reduced pressure to provide a brown solid. This material was dried under high vacuum to provide 2-ethxoymethyl-N-phenylmethylidine-1H-imidazo[4,5-c][1,5]naphthyridin-1-amine.

### Part B

A suspension of the material from Part A in methanol (30 mL) was warmed to bring the material into solution. The solution was slowly cooled and then placed in an ice bath. Sodium borohydride (0.23 g, 2 eq) was added. After 30 minutes the reaction mixture was allowed to slowly come to ambient temperature. After 45 minutes the reaction mixture was quenched with the dropwise addition of saturated ammonium chloride (5 mL) and then concentrated under reduced pressure to provide a yellow solid. This material was partitioned between 10% sodium carbonate and chloroform (30 mL). The organic layer was separated, washed sequentially with water (10 mL) and brine (10 mL), dried over sodium sulfate, filtered, and then concentrated under reduced pressure to provide 0.99 g of N-benzyl-2-ethxoymethyl-1H-imidazo[4,5-c][1,5]naphthyridin-1-amine as a light yellow solid.

### Part C

A solution of the material from Part B in 1,2-dichloroethane (30 mL) was placed under a nitrogen atmosphere in a pressure vessel. 3-Chloroperoxybenzoic acid (0.73 g, 1 eq) was added and the reaction mixture was stirred at ambient temperature for 1.5 hours. Two additional portions of 3-chloroperoxybenzoic acid (0.25 g) were added 30 minutes apart and stirring was continued for 30 minutes after the second addition. Ammonium hydroxide (10 mL of 30%) was added. The vessel was sealed and heated to 50° C. para-Toluenesulfonyl chloride (0.59 g, 1.05 eq) was added. The vessel was sealed and stirred vigorously at 70° C. for 30 minutes. The reaction mixture was cooled to ambient temperature and then filtered. The filtrate was diluted with chloroform (30 mL) and water (10 mL) and then shaken. The layers were separated. The organic layer was washed sequentially with 10% sodium carbonate (20 mL) and water (20 mL). The combined aqueous washes were back extracted with chloroform (20 mL). The combined organics were washed with brine (20 mL), dried over sodium sulfate, filtered, and then concentrated under reduced pressure to provide 1 g of a yellow solid. This material was purified by HPFC (100 g of silica gel eluting with a gradient of 1-15% CMA in chloroform) to provide a beige solid. The solid was recrystallized from acetonitrile to provide 0.20 g of N¹-benzyl-2-ethxoymethyl-1H-imidazo[4,5-c][1,5]naphthyridine-1,4-diamine as an off-white solid, mp 169-171° C. ¹H NMR (500 MHz, DMSO-d₆) δ 8.63 (dd, J=4.3, 1.6 Hz, 1H), 7.95 (dd, J=8.4, 1.5 Hz, 1H), 7.51 (dd, J=8.4, 4.3 Hz, 1H), 7.39-7.37 (m, 3H), 7.35-7.30 (m, 2H), 7.08 (t, J=5.7 Hz, 1H), 6.93 (s, 2H), 4.51 (s, 2H), 4.43 (d, J=5.7 Hz, 2H), 3.54 (q, J=7.0 Hz, 2H), 1.13 (t, J=7.0 Hz, 3H); ¹³C NMR (125 MHz, DMSO-d₆) δ 152.7, 150.3, 143.9, 140.5, 137.1, 133.4, 133.0, 131.6, 129.8, 128.8, 128.1, 127.5, 122.8, 65.9, 62.8, 56.7, 15.4; MS (ESI) m/z 349.34 (M+H)⁺; Anal. Calcd for C₁₉H₂₀N₆O: C, 65.50; H, 5.79; N, 24.12; Found: C, 65.44; H, 5.59; N, 23.94.

### Example 35

### (4-Amino-1-benzylamino-1H-imidazo[4,5-c][1,5]naphthyridin-2-yl)methanol

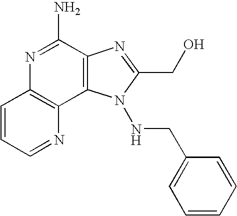

Under a nitrogen atmosphere boron tribromide (1.03 mL of 1 M in dichloromethane, 2 eq) was added dropwise to a chilled (ice water bath) solution of N¹-benzyl-2-ethxoymethyl-1H-imidazo[4,5-c][1,5]naphthyridine-1,4-diamine (0.18 g, 1 eq) in dichloromethane (15 mL). The reaction was allowed to slowly come to ambient temperature. After 6 hours additional boron tribromide (0.50 mL) was added and the reaction mixture was stirred at ambient temperature over the weekend. The reaction mixture was quenched with the dropwise addition of water (2 mL) and then it was concentrated under reduced pressure to provide a tan solid. The solid was combined with a solution of ammonia in methanol (10 mL of 7 M) and stirred for 1 hour. Silica gel (3 g) was added. The mixture was stirred for 5 minutes, concentrated under reduced pressure, and then loaded onto a HPFC column. The column was eluted with a gradient of 1-25% CMA in chloroform. The resulting beige solid was triturated with ether, isolated by filtration, and then dried under vacuum at 80° C. to provide 23 mg of (4-amino-1-benzylamino-1H-imidazo[4,5-c][1,5]naphthyridin-2-yl)methanol as a beige solid, mp 237-239° C. ¹H NMR (500 MHz, DMSO-d₆) δ 8.62 (dd, J=4.3, 1.5 Hz, 1H), 7.95 (dd, J=8.3, 1.5 Hz, 1H), 7.50 (dd, J=8.4, 4.3 Hz, 1H), 7.40-7.30 (m, 5H), 7.01 (t, J=5.8 Hz, 1H), 6.87 (s, 2H), 5.38 (t, J=5.9 Hz, 1H), 4.52 (d, J=5.9 Hz, 2H), 4.44 (d, J=5.8 Hz, 2H); ¹³C NMR (125 MHz, DMSO-d₆) δ 152.8, 152.2, 143.4, 139.9, 13.67, 133.0, 132.4, 131.0, 129.4, 128.3, 127.6, 126.9, 122.2, 56.2, 54.5; MS (ESI) m/z 321.26 (M+H)⁺; Anal. Calcd for C₁₇H₁₆N₆O: C, 63.74; H, 5.03; N, 26.23; Found: C, 63.52; H, 4.74; N, 26.05.

### Example 36

### 2-Ethxoymethyl-N-(tetrahydro-2H-pyran-4-yl)-1H-imidazo[4,5-c][1,5]naphthyridin-1-amine

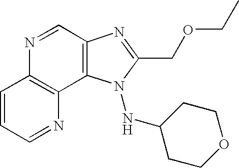

2-Ethxoymethyl-N-(tetrahydro-2H-pyran-4-yl)-1H-imidazo[4,5-c][1,5]naphthyridin-1-amine was prepared according to the general methods of Example 34 Parts A and B using tetrahydro-4H-pyran-4-one in lieu of benzaldehyde in Part A. The crude product was purified by HPFC (100 g of silica gel eluted with a gradient of 1-10% CMA in chloroform) to provide an off white solid. The solid was triturated with ether, isolated by filtration, and then dried under vacuum at 70° C. to provide 1.64 g of 2-ethxoymethyl-N-(tetrahydro-2H-pyran-4-yl)-1H-imidazo[4,5-c][1,5]naphthyridin-1-amine as a white solid, mp 130-133° C. ¹H NMR (300 MHz, DMSO-d₆) δ 9.29 (s, 1H), 9.05 (dd, J=4.2, 1.6 Hz, 1H), 8.55 (dd, J=8.5, 1.6 Hz, 1H), 7.78 (dd, J=8.5, 4.3 Hz, 1H), 7.03 (d, J=2.3 Hz, 1H), 4.82 (s, 2H), 3.92-3.77 (m, 3H), 3.68 (q, J=7.0 Hz, 2H), 3.27-3.19 (m, 2H), 1.75-1.48 (m, 4H), 1.17 (t, J=7.0 Hz, 3H); ¹³C NMR (75 MHz, DMSO-d₆) δ 153.5, 150.1, 146.1, 139.3, 137.7, 137.5, 134.7, 132.6, 123.1, 66.1, 66.7, 56.8, 31.0, 15.4; MS (ESI) m/z 328.32 (M+H)⁺; Anal. Calcd for C₁₇H₂₁N₅O₂: C, 62.37; H, 6.47; N, 21.39; Found: C, 62.39; H, 6.45; N, 21.37.

### Example 37

### 2-Ethxoymethyl-N¹-(tetrahydro-2H-pyran-4-yl)-1H-imidazo[4,5-c][1,5]naphthyridine-1,4-diamine

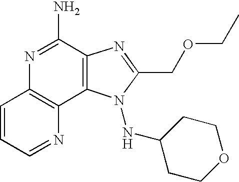

2-Ethxoymethyl-N-(tetrahydro-2H-pyran-4-yl)-1H-imidazo[4,5-c][1,5]naphthyridin-1-amine (1.00 g) was oxidized and then aminated using the methods of Example 34 Part C. The crude product was purified by HPFC (100 g of silica gel eluted with a gradient of 1-15% CMA in chloroform) to provide 0.49 g of 2-ethxoymethyl-N¹-(tetrahydro-2H-pyran-4-yl)-1H-imidazo[4,5-c][1,5]naphthyridine-1,4-diamine as a pale yellow solid, mp 191-193° C. ¹H NMR (300 MHz, DMSO-d₆) δ 8.55 (dd, J=4.3, 1.5 Hz, 1H), 7.93 (dd, J=8.4, 1.5 Hz, 1H), 7.47 (dd, J=8.4, 4.3 Hz, 1H), 6.95 (s, 2H), 6.89 (d, J=2.5 Hz, 1H), 4.74 (s, 2H), 3.91-3.79 (m, 2H), 3.79-3.69 (m, 1H), 3.63 (q, J=7.0 Hz, 2H), 3.26-3.19 (m, 2H), 1.73-1.46 (m, 4H), 1.16 (t, J=7.0 Hz, 3H); ¹³C NMR (75 MHz, DMSO-d₆) δ 152.7, 150.8, 143.8, 140.5, 133.5, 132.9, 131.6, 127.6, 122.7, 65.9, 65.7, 62.5, 56.9, 31.0, 15.4; MS (APCI) m/z 343.20 (M+H)⁺; Anal. Calcd for C₁₇H₂₂N₆O₂: C, 59.63; H, 6.48; N, 24.54; Found: C, 59.37; H, 6.62; N, 24.41.

### Example 38

### [1-(Tetrahydro-2H-pyran-4-yl)amino-1H-imidazo[4,5-c][1,5]naphthyridin-2-yl]methanol

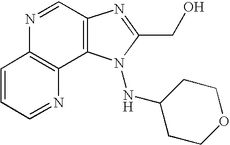

Under a nitrogen atmosphere boron tribromide (2.00 mL of 1 M in dichloromethane, 2 eq) was added dropwise to a chilled (ice water bath) solution of 2-ethxoymethyl-N-(tetrahydro-2H-pyran-4-yl)-1H-imidazo[4,5-c][1,5]naphthyridin-1-amine (0.327 g, 1 eq) in dichloromethane (10 mL). The reaction was allowed to slowly come to ambient temperature and was stirred overnight. After 18 hours the reaction was quenched with the dropwise addition of water (2 mL) and methanol (10 mL) was added. The dichloromethane and methanol were removed under reduced pressure to provide an aqueous slurry. A solution of ammonia in methanol (10 mL of 7 M) was added and the mixture was stirred for 1 hour. Silica gel (3 g) was added and the slurry was loaded on a HPFC column which was then eluted with a gradient of 1-30% CMA in chloroform to provide a yellow solid. The solid was purified by HPFC (40 g of silica gel eluted with a gradient of 1-25% CMA in chloroform) to provide 15 mg of a light yellow solid. This material was recrystallized from acetonitrile to provide 5 mg of [1-(tetrahydro-2H-pyran-4-yl)amino-1H-imidazo[4,5-c][1,5]naphthyridin-2-yl]methanol as light yellow crystals, mp 203-205° C. ¹H NMR (500 MHz, DMSO-d₆) δ 9.26 (s, 1H), 9.04 (dd, J=4.2, 1.6 Hz, 1H), 8.53 (dd, J=8.5, 1.6 Hz, 1H), 7.76 (dd, J=8.5, 4.2 Hz, 1H), 6.96 (d, J=2.5 Hz, 1H), 5.56 (t, J=6.1 Hz, 1H), 4.83 (d, J=6.1 Hz, 2H), 3.88-3.82 (m, 2H), 3.82-3.75 (m, 1H), 3.24-3.20 (m, 2H), 1.65 (br, 2H), 1.57-1.50 (m, 2H); ¹³C NMR (125 MHz, DMSO-d₆) δ 156.1, 149.6, 145.5, 138.7, 137.3, 137.1, 134.3, 132.1, 122.5, 65.2, 56.4, 54.7, 30.6; MS (APCI) m/z 300.17 (M+H)⁺; Anal. Calcd for C₁₅H₁₇N₅O₂: C, 60.19; H, 5.72; N, 23.40; Found: C, 59.91; H, 5.41; N, 23.05.

### Example 39

### 2-(Ethoxymethyl)-6,7-dimethyl-N¹-[3-(methylsulfonyl)propyl]-1H-imidazo[4,5-c]pyridine-1,4-diamine

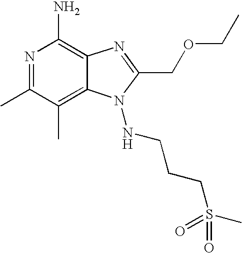

### Part A

3-(Methylthio)propionaldehyde (0.9 mL, 1.1 eq) was added to a warm (30-40° C.) solution of 4-chloro-2-(ethoxymethyl)-6,7-dimethyl-1H-imidazo[4,5-c]pyridin-1-amine (2.0 g, 1 eq) in glacial acetic acid (20 mL) and acetonitrile (20 mL). The solution was heated at 100° C. for 2 hours and then at 90° C. over night. Additional 3-(methylthio)propionaldehyde (0.3 mL) was added and the reaction mixture was heated for several more hours. The reaction mixture was concentrated under reduced pressure and the residue was partitioned between 10% aqueous sodium carbonate and dichloromethane. The layers were separated and the aqueous layer was extracted with dichloromethane (×2). The combined organics were washed sequentially with water and brine, dried over sodium sulfate, filtered, and then concentrated under reduced pressure to provide crude 4-chloro-2-(ethoxymethyl)-6,7-dimethyl-N-[(1E)-3-(metaylthio)propylidene]-1H-imidazo[4,5-c]pyridin-1-amine as a brown oil.

### Part B

Sodium borohydride (0.9 g, 3 eq) was added to a chilled (0° C.) solution of the material from Part A (1 eq) in methanol (50 mL). The reaction mixture was stirred at 0° C. for 2 hours and then it was quenched with saturated aqueous ammonium chloride. The methanol was removed under reduced pressure and the residue was extracted with dichloromethane (×3). The combined organics were washed sequentially with water and brine, dried over sodium sulfate, filtered, and then concentrated under reduced pressure to provide a brown oil. This material was combined with material from another run and purified by HPFC (silica gel eluted with a gradient of 20-80% ethyl acetate in hexanes) to provide 1.65 g of 4-chloro-2-(ethoxymethyl)-6,7-dimethyl-N-[3-(methylthio)propyl]-1H-imidazo[4,5-c]pyridin-1-amine as a pale pink oil which solidified on standing.

### Part C

3-Chloroperoxybenzoic acid (2 g of 77%, 2 eq) was added over a period of 2 minutes to a chilled (0° C.) solution of 4-chloro-2-(ethoxymethyl)-6,7-dimethyl-N-[3-(methylthio)propyl]-1H-imidazo[4,5-c]pyridin-1-amine (1.52 g, 1 eq) in dichloromethane (50 mL). The reaction mixture was allowed to slowly warm to ambient temperature. After 3 hours the reaction mixture was diluted with dichloromethane and then washed sequentially with 10% sodium carbonate (×2), water, and brine. The organic layer was dried over sodium sulfate, filtered, and then concentrated under reduced pressure to provide 1.17 g of 4-chloro-2-(ethoxymethyl)-6,7-dimethyl-N-[3-(methylsulfonyl)propyl]-1H-imidazo[4,5-c]pyridin-1-amine as a clear oil. This material was concentrated from toluene and then carried on to the next step.

### Part D

The material from Part C (1 eq) was combined with 4-methoxybenzylamine (4.08 mL, 10 eq), pyridine hydrochloride (1.80 g, 5 eq), and 2,2,2-trifluoroethanol (10 mL) and heated in a microwave at 160° C. for 2 hours. The reaction mixture was allowed to cool to ambient temperature and then it was concentrated under reduced pressure. The residue was partitioned between ethyl acetate and 10% sodium carbonate. The organic phase was washed sequentially with 10% sodium carbonate (×2), water, and brine. The organic layer was dried over sodium sulfate, filtered, and then concentrated under reduced pressure to provide crude 2-(ethoxymethyl)-N-(4-methoxybenzyl)-6,7-dimethyl-N¹-[3-(methylsulfonyl)propyl]-1H-imidazo[4,5-c]pyridine-1,4-diamine.

### Part E

Trifluoroacetic acid (30 mL) was added to a chilled (0° C.) solution of the material from Part D in dichloromethane (20 mL). The resulting solution was allowed to stand at ambient temperature over night and then it was concentrated under reduced pressure. The oily residue was combined with 10% aqueous sodium hydroxide (50 mL) and then extracted with dichloromethane (×3). The combined organics were washed sequentially with water and brine (×2), dried over sodium sulfate, filtered, and then concentrated under reduced pressure. The residue was triturated with toluene to provide a solid. The solid was isolated by filtration and then triturated with toluene (×4) to provide 0.57 g of 2-(ethoxymethyl)-6,7-dimethyl-N¹-[3-(methylsulfonyl)propyl]-1H-imidazo[4,5-c]pyridine-1,4-diamine as a white powder, mp 150-155° C. ¹H NMR (300 MHz, CDCl₃) δ 5.36 (t, J=6.5 Hz, 1H), 4.88 (br s, 2H), 4.76 (s, 2H), 3.62 (q, J=6.9 Hz, 2H), 3.34 (q, J=6.9, 2H), 3.20 (m, 2H), 2.96 (s, 3H), 2.49 (s, 3H), 2.42 (s, 3H), 2.18 (pentet, J=7.5 Hz, 2H), 1.24 (t, J=6.9 Hz, 3H); MS (ESI) m/z 356 (M+H)⁺; Anal. calcd for C₁₅H₂₅N₅O₃S: C, 50.69; H, 7.09; N, 19.70; S, 9.02. Found: C, 50.65; H, 6.97; N, 19.49; S, 9.37.

### Example 40

### 2-(Ethoxymethyl)-6,7-dimethyl-N¹-[1-(methylsulfonyl)piperidin-4-yl]-1H-imidazo[4,5-c]pyridine-1,4-diamine

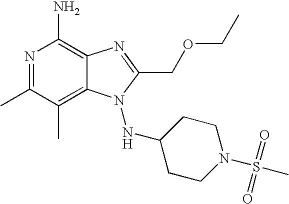

### Part A

Trifluoroacetic acid (60 mL) was added to a chilled (0° C.) solution of tert-butyl 4-oxopiperidine-1-carboxylate (2.00 g) in dichloromethane (60 mL). The resulting solution was allowed to warm to ambient temperature. After 4.5 hours the reaction mixture was concentrated under reduced pressure to provide an oil. The oil was concentrated twice from toluene to provide piperidin-4-one trifluoroacetate as a yellow-white solid.

### Part B

Triethylamine (2.79 mL, 2 eq) was added to a chilled (0° C.) suspension of the material from Part A (1 eq) in dichloromethane (40 mL). Additional dichloromethane (20 mL) was added to bring all of the material into solution. Methanesulfonic anhydride (1.72 g, 1 eq) was added in a single portion. The progress of the reaction was monitored by thin layer chromatography. Additional triethylamine (1 mL) and methanesulfonic anhydride (0.4 g) were added. After 4 hours the reaction mixture was diluted with methanol and then concentrated under reduced pressure to provide 1-(methylsulfonyl)piperidin-4-one as an oil.

### Part C

The material from Part B was combined with 4-chloro-2-(ethoxymethyl)-6,7-dimethyl-1H-imidazo[4,5-c]pyridin-1-amine (1.28 g), glacial acetic acid (20 mL), and acetonitrile (20 mL) and heated at 90° C. overnight. The reaction mixture was concentrated under reduced pressure and the residue was partitioned between 10% aqueous sodium carbonate and dichloromethane. The layers were separated and the aqueous layer was extracted with dichloromethane (×2). The combined organics were washed sequentially with water and brine, dried over sodium sulfate, filtered, and then concentrated under reduced pressure to provide crude 4-chloro-2-(ethoxymethyl)-6,7-dimethyl-N-[1-(methylsulfonyl)piperidin-4-ylidene]-1H-imidazo[4,5-c]pyridin-1-amine.

### Part D

Sodium borohydride (1 g) was added in portions over a period of 2 hours to a solution of the material from Part C in methanol (50 mL). The reaction mixture was quenched with saturated aqueous ammonium chloride. The methanol was removed under reduced pressure and the residue was extracted with dichloromethane (×3). The combined organics were washed sequentially with water and brine, dried over sodium sulfate, filtered, and then concentrated under reduced pressure. The crude product was purified by HPFC (silica gel eluted with a gradient of 2-15% methanol in dichloromethane) to provide 4-chloro-2-(ethoxymethyl)-6,7-dimethyl-N-[1-(methylsulfonyl)piperidin-4-yl]-1H-imidazo[4,5-c]pyridin-1-amine.

### Part E

The material from Part D was concentrated twice from toluene and then combined with 4-methoxybenzylamine (6.5 mL, 10 eq), pyridine hydrochloride (2.89 g, 5 eq), and 2,2,2-trifluoroethanol (16 mL) and heated in a microwave at 160° C. for 2 hours. The reaction mixture was allowed to cool to ambient temperature and then it was concentrated under reduced pressure. The residue was dissolved in dichloromethane and the solution was washed sequentially with 10% sodium carbonate (×2), water, and brine. The organic layer was dried over sodium sulfate, filtered, and then concentrated under reduced pressure to provide crude 2-(ethoxymethyl)-N⁴-(4-methoxybenzyl)-6,7-dimethyl-N¹-[1-(methylsulfonyl)piperidin-4-yl]-1H-imidazo[4,5-c]pyridine-1,4-diamine.

### Part F

Trifluoroacetic acid (30 mL) was added to a cold solution of the material from Part E in dichloromethane (15 mL). The reaction mixture was allowed to warm to ambient temperature and then to stand overnight. The reaction mixture was concentrated under reduced pressure to provide an oil. The oil was diluted with dichloromethane and then washed sequentially with 10% aqueous sodium hydroxide, water, and brine. The organic layer was dried over sodium sulfate, filtered, and then concentrated under reduced pressure to provide a yellow-brown oil. The oil was combined with toluene and the mixture was chilled for 2 hours. A solid was isolated by filtration to provide a first crop (0.22 g). A precipitate formed in the filtrate and was isolated by filtration to provide a second crop (0.80 g). The combined crops were recrystallized first from acetonitrile and then from ethanol to provide 0.45 g of 2-(ethoxymethyl)-6,7-dimethyl-N¹-[1-(methylsulfonyl)piperidin-4-yl]-1H-imidazo[4,5-c]pyridine-1,4-diamine as white crystals, mp 127-131° C. ¹H NMR (500 MHz, CDCl₃) δ 5.35 (d, J=2.8 Hz, 1H), 4.87 (br s, 2H), 4.83-4.74 (br s, 2H), 3.84 (m, 2H), 3.62 (q, J=6.9 Hz, 2H), 3.25 (m, 1H), 2.79 (s, 3H), 2.71-2.66 (m, 2H), 2.49 (s, 3H), 2.42 (s, 3H), 1.82-1.67 (m, 4H), 1.25 (t, J=6.9 Hz, 3H); HRMS (ESI) calcd for C₁₇H₂₈N₆O₃S+H 397.2022, found 397.2030.

### Example 41

### N¹-(1-Acetylpiperidin-4-yl)-2-(ethoxymethyl)-6,7-dimethyl-1H-imidazo[4,5-c]pyridine-1,4-diamine

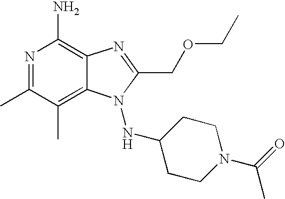

N¹-(1-Acetylpiperidin-4-yl)-2-(ethoxymethyl)-6,7-dimethyl-1H-imidazo[4,5-c]pyridine-1,4-diamine was prepared according to the methods of Example 40 using acetic anhydride in lieu of methanesulfonic anhydride in Part B. The crude product was triturated with toluene to provide a solid. The solid was recrystallized twice from acetonitrile to provide N¹-(1-acetylpiperidin-4-yl)-2-(ethoxymethyl)-6,7-dimethyl-1H-imidazo[4,5-c]pyridine-1,4-diamine as a white solid, HRMS (ESI) calcd for C₁₈H₂₈N₆O₂+H 361.2352, found 361.2361.

### Example 42

### {4-Amino-2-(ethoxymethyl)-6,7-dimethyl-1-[3-(methylsulfonyl)propyl]amino-1H-imidazo[4,5-c]pyridin-2-yl}methanol

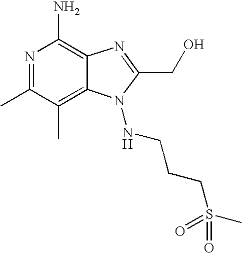

The ether group on 2-(ethoxymethyl)-6,7-dimethyl-N¹-[3-(methylsulfonyl)propyl]-1H-imidazo[4,5-c]pyridine-1,4-diamine is cleaved using boron tribromide to provide {4-amino-2-(ethoxymethyl)-6,7-dimethyl-1-[3-(methylsulfonyl)propyl]amino-1H-imidazo[4,5-c]pyridin-2-yl}methanol.

### Example 43

### {4-Amino-2-(ethoxymethyl)-6,7-dimethyl-1-[1-(methylsulfonyl)piperidin-4-yl]amino-1H-imidazo[4,5-c]pyridin-2-yl}methanol

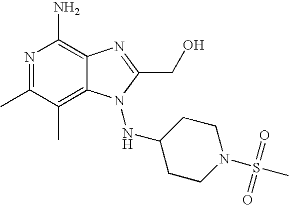

The ether group on 2-(ethoxymethyl)-6,7-dimethyl-N¹-[1-(methylsulfonyl)piperidin-4-yl]-1H-imidazo[4,5-c]pyridine-1,4-diamine is cleaved using boron tribromide to provide {4-amino-2-(ethoxymethyl)-6,7-dimethyl-1-[1-(methylsulfonyl)piperidin-4-yl]amino-1H-imidazo[4,5-c]pyridin-2-yl}methanol.

### Example 44

### {1-(1-Acetylpiperidin-4-yl)amino-4-amino-2-(ethoxymethyl)-6,7-dimethyl-1H-imidazo[4,5-c]pyridin-2-yl}methanol

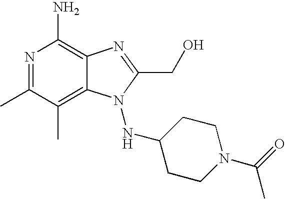

The ether group on N¹-(1-acetylpiperidin-4-yl)-2-(ethoxymethyl)-6,7-dimethyl-1H-imidazo[4,5-c]pyridine-1,4-diamine is cleaved using boron tribromide to provide {1-(1-acetylpiperidin-4-yl)amino-4-amino-2-(ethoxymethyl)-6,7-dimethyl-1H-imidazo[4,5-c]pyridin-2-yl}methanol.

### Example 45

### N¹-(3,4-Dichlorobenzyl)-2-ethoxymethyl-6,7-dimethyl-1H-imidazo[4,5-c]pyridine-1,4-diamine

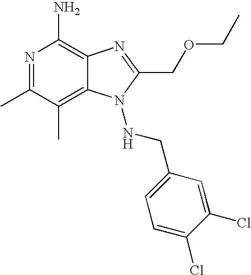

N¹-(3,4-Dichlorobenzyl)-2-ethoxymethyl-6,7-dimethyl-1H-imidazo[4,5-c]pyridine-1,4-diamine was prepared using a modification of the methods of Example 25. 3,4-Dichlorobenzaldehyde was used in lieu of tetrahydro-4H-pyran-4-one in Part A and the 4-methoxybenzyl group was installed using the method of Part A of Example 21. The crude product was purified by HPFC (silica gel eluted with a gradient of 5-20% methanol in dichloromethane) followed by recrystallization from ethanol to provide N¹-(3,4-dichlorobenzyl)-2-ethoxymethyl-6,7-dimethyl-1H-imidazo[4,5-c]pyridine-1,4-diamine as white needles, mp 186-188° C. ¹H NMR (500 MHz, DMSO-d₆) δ 7.63 (d, J=8.2 Hz, 1H), 7.60 (d, J=1.9 Hz, 1H), 7.30 (dd, J=8.2, 2.0 Hz, 1H), 6.92 (t, J=5.8 Hz, 1H), 5.81 (br s, 2H), 4.54 (s, 2H), 4.19 (d, J=5.8 Hz, 2H), 3.54 (q, J=7.0 Hz, 2H), 2.45 (s, 3H), 2.29 (s, 3H), 1.12 (t, J=7.0 Hz, 3H); MS (ESI) m/z 394 (M+H)⁺; 396 (M+H+2)⁺; Anal. Calcd for C₁₈H₂₁Cl₂N₅O: C, 54.83; H, 5.37; N, 17.98. Found: C, 55.01; H, 5.39; N, 17.80.

### Example 46

### N¹-Cyclopentyl-2-ethoxymethyl-6,7-dimethyl-1H-imidazo[4,5-c]pyridine-1,4-diamine

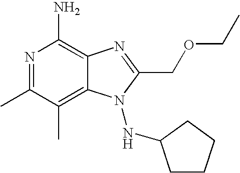

N¹-Cyclopentyl-2-ethoxymethyl-6,7-dimethyl-1H-imidazo[4,5-c]pyridine-1,4-diamine was prepared using a modification of the methods of Example 25. Cyclopentanone was used in lieu of tetrahydro-4H-pyran-4-one in Part A and the 4-methoxybenzyl group was installed using the method of Part A of Example 21. The crude product was purified by HPFC (silica gel eluted with a gradient of 10-30% methanol in dichloromethane) followed by recrystallization from acetonitrile to provide N¹-cyclopentyl-2-ethoxymethyl-6,7-dimethyl-1H-imidazo[4,5-c]pyridine-1,4-diamine as white crystals, mp 130-132° C. ¹H NMR (300 MHz, DMSO-d₆) δ 6.43 (d, J=1.1 Hz, 1H), 5.78 (br s, 2H), 4.62 (s, 2H), 3.70 (m, 1H), 3.58 (q, J=7.0 Hz, 2H), 2.43 (s, 3H), 2.28 (s, 3H), 1.82-1.36 (m, 8H), 1.14 (t, J=7.0 Hz, 3H); MS (ESI) m/z 304 (M+H)⁺; Anal. Calcd for C₁₆H₂₅N₅O: C, 63.34; H, 8.31; N, 23.08. Found: C, 63.27; H, 8.47; N, 23.40.

### Example 47

### (4-Amino-1-cyclopentylamino-6,7-dimethyl-1H-imidazo[4,5-c]pyridin-2-yl)methanol

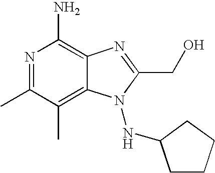

The ether group on N¹-cyclopentyl-2-ethoxymethyl-6,7-dimethyl-1H-imidazo[4,5-c]pyridine-1,4-diamine was cleaved using the method of Example 12. The crude product was purified by HPFC (silica gel eluted with a gradient of 5-20% methanol in dichloromethane containing 2% ammonium hydroxide) followed by recrystallization from DMF to provide a white solid. This material was dissolved in a mixture of methanol and dichloromethane and the solution was concentrated under reduced pressure to provide (4-amino-1-cyclopentylamino-6,7-dimethyl-1H-imidazo[4,5-c]pyridin-2-yl)methanol as a white solid, mp 254-256° C. ¹H NMR (300 MHz, DMSO-d₆) δ 6.35 (d, J=1.5 Hz, 1H), 5.70 (br s, 2H), 5.42 (t, J=5.8 Hz, 1H), 4.66 (d, J=5.6 Hz, 2H), 3.74 (m, 1H), 2.43 (s, 3H), 2.27 (s, 3H), 1.82-1.35 (m, 8H); MS (ESI) m/z 276 (M+H)⁺; Anal. Calcd for C₁₄H₂₁N₅O: C, 61.07; H, 7.69; N, 25.43. Found: C, 60.88; H, 7.58; N, 25.53.

### Example 48

### 2-Ethyl-6,7-dimethyl-N¹-(tetrahydropyran-4-yl)-1H-imidazo[4,5-c]pyridine-1,4-diamine

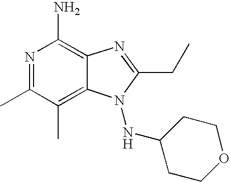

2-Ethyl-6,7-dimethyl-N¹-(tetrahydropyran-4-yl)-1H-imidazo[4,5-c]pyridine-1,4-diamine was prepared according to the methods of Example 28 using triethyl orthopropionate in lieu of triethyl orthoacetate in Part A and tetrahydro-4H-pyran-4-one in lieu of cyclohexanone in Part C. The crude product was purified twice by HPFC (silica gel eluted with a gradient of 10-30% methanol in dichloromethane) and then recrystallized from water to provide 2-ethyl-6,7-dimethyl-N¹-(tetrahydropyran-4-yl)-1H-imidazo[4,5-c]pyridine-1,4-diamine as white crystals, mp 191-193° C. ¹H NMR (300 MHz, DMSO-d₆) δ 6.61 (d, J=1.3 Hz, 1H), 5.59 (br s, 2H), 3.81 (m, 2H), 3.44-3.10 (m, 3H), 3.05-2.67 (m, 2H), 2.42 (s, 3H), 2.27 (s, 3H), 1.62-1.25 (m, 4H), 1.30 (t, J=7.5 Hz, 3H); MS (ESI) m/z 290 (M+H)⁺; Anal. Calcd for C₁₅H₂₃N₅O.0.25H₂O: C, 61.30; H, 8.06; N, 23.83. Found: C, 61.31; H, 8.09; N, 24.10.

### Example 49

### Ethyl [3-(4-Amino-2-ethoxymethyl-6,7-dimethyl-1H-imidazo[4,5-c]pyridin-1-yl)propyl)carbamate

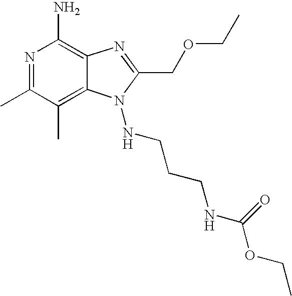

### Part A

Tert-butyl {3-[(4-chloro-2-ethoxymethyl-6,7-dimethyl-1H-imidazo[4,5-c]pyridin-1-yl)amino]propyl}carbamate (2.41 g) was converted to tert-butyl {3-[(4-benzylamino-2-ethoxymethyl-6,7-dimethyl-1H-imidazo[4,5-c]pyridin-1-yl)amino]propyl}carbamate (2.82 g) according to the method of Example 21 Part A using benzylamine in lieu of 4-methoxybenzylamine.

### Part B

The tert-butoxycarbonyl group was removed from the material from Part A using the method of Example 13 Part F to provide N¹-(3-aminopropyl)-N⁴-benzyl-2-ethoxymethyl-6,7-dimethyl-1H-imidazo[4,5-c]pyridine-1,4-diamine (1.57 g).

### Part C

The material from Part B was reacted with ethyl chloroformate according to the method of Example 13 Part G using ethyl chloroformate in lieu of methanesulfonyl chloride to provide ethyl [3-(4-benzylamino-2-ethoxymethyl-6,7-dimethyl-1H-imidazo[4,5-c]pyridin-1-yl)propyl)carbamate (1.71 g).

### Part D

The benzyl group was removed from the material from Part D using the method of Example 13 Part I. The crude product was purified by HPFC (silica gel eluted with a gradient of 10-30% methanol in dichloromethane) to provide 0.57 g of ethyl [3-(4-amino-2-ethoxymethyl-6,7-dimethyl-1H-imidazo[4,5-c]pyridin-1-yl)propyl)carbamate as a white solid, mp 148-150° C. ¹H NMR (300 MHz, CDCl₃) δ 5.22 (t, J=6.7 Hz, 1H), 5.17 (m, 1H), 4.92 (br s, 2H), 4.76 (s, 2H), 4.12 (q, J=7.1 Hz, 2H), 3.61 (q, J=7.0 Hz, 2H), 3.37 (q, J=6.3 Hz, 2H), 3.20 (q, J=6.7 Hz, 2H), 2.48 (s, 3H), 2.42 (s, 3H), 1.81 (pentet, J=6.6 Hz, 2H), 1.30-1.20 (m, 6H); MS (ESI) m/z 365 (M+H)⁺; Anal. Calcd for C₁₇H₂₈N₆O₃: C, 56.03; H, 7.74; N, 23.06. Found: C, 56.21; H, 7.54; N, 23.19.

### Example 50

### 2-[4-Amino-6,7-dimethyl-1-(tetrahydropyran-4-yl)amino-1H-imidazo[4,5-c]pyridin-2-yl]ethanol

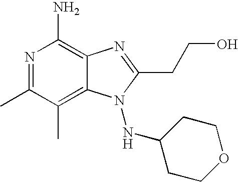

### Part A

Tert-butyl[4-chloro-2-(2-methoxyethyl)-6,7-dimethyl-1H-imidazo[4,5-c]pyridin-1-yl]carbamate (5.08 g) was prepared according to the method of Example 33 Part A using 3-methoxypropionyl chloride in lieu of butyryl chloride.

### Part B

Under a nitrogen atmosphere boron tribromide (14.2 mL, 3 eq) was added dropwise to a chilled (ice bath) solution of tert-butyl[4-chloro-2-(2-methoxyethyl)-6,7-dimethyl-1H-imidazo[4,5-c]pyridin-1-yl]carbamate (2.01 g, 1 eq) in dichloromethane (40 mL). The reaction mixture was allowed to warm to ambient temperature after 30 minutes. After 16 hours the reaction was quenched with methanol (20 mL) and stirred for 20 minutes. Hydrochloric acid (20 mL of 6 N) was added and the reaction mixture was heated at 40° C. for 2 hours. The reaction mixture was stirred at ambient temperature overnight and then the pH was adjusted to 13 with 50% sodium hydroxide. The reaction mixture was extracted with dichloromethane (10×100 mL). The aqueous phase was placed in a continuous extractor and extracted overnight with chloroform. The combined organics were concentrated under reduced pressure to provide an amber oil. The oil was purified by HPFC (silica gel eluted with a gradient of 10-30% methanol in dichloromethane) to provide 0.66 g of 2-(1-amino-4-chloro-6,7-dimethyl-1H-imidazo[4,5-c]pyridin-2-yl)ethanol as a brown foamy solid.

### Part C

The material from Part B was converted to 2-[4-amino-6,7-dimethyl-1-(tetrahydropyran-4-yl)amino-1H-imidazo[4,5-c]pyridin-2-yl]ethanol according to the methods of Example 28 Parts C through F using tetrahydro-4H-pyran-4-one in lieu of cyclohexanone in Part C. The crude product was purified by HPFC (silica gel eluted with a gradient of 10-30% methanol in dichloromethane containing 2% concentrated ammonium hydroxide) followed by recrystallization from acetonitrile to provide 0.14 g of 2-[4-amino-6,7-dimethyl-1-(tetrahydropyran-4-yl)amino-1H-imidazo[4,5-c]pyridin-2-yl]ethanol as white needles, mp 190-192° C. ¹H NMR (500 MHz, DMSO-d₆) δ 6.66 (d, J=1.8 Hz, 1H), 5.63 (br s, 2H), 4.87 (t, J=5.4 Hz, 1H), 3.82 (m, 4H), 3.29-2.83 (m, 5H), 2.42 (s, 3H), 2.27 (s, 3H), 1.54-1.30 (m, 4H); MS (ESI) m/z 306 (M+H)⁺; Anal. Calcd for C₁₅H₂₃N₅O₂: C, 59.00; H, 7.59; N, 22.93. Found: C, 59.17; H, 7.51; N, 23.08.

### Example 51

### 2-(2-Methoxyethyl)-6,7-dimethyl-N¹-(tetrahydropyran-4-yl)-1H-imidazo[4,5-c]pyridine-1,4-diamine

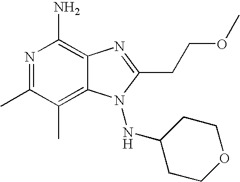

### Part A

The tert-butoxycarbonyl group was removed from tert-butyl[4-chloro-2-(2-methoxyethyl)-6,7-dimethyl-1H-imidazo[4,5-c]pyridin-1-yl]carbamate ((2.0 g) using the general method of Example 13 Part C to provide 4-chloro-2-(2-methoxyethyl)-6,7-dimethyl-1H-imidazo[4,5-c]pyridin-1-amine (0.91 g).

### Part B

The material from Part B was converted to 4-chloro-2-(2-methoxyethyl)-6,7-dimethyl-N-(tetrahydropyran-4-yl)-1H-imidazo[4,5-c]pyridin-1-amine (0.84 g) using the methods of Example 25 Parts A and B.

### Part C

The material from Part C was converted to N⁴-benzyl-2-(2-methoxyethyl)-6,7-dimethyl-N¹-(tetrahydropyran-4-yl)-1H-imidazo[4,5-c]pyridine-1,4-diamine (0.57 g) according to the method of Example 21 Part A using benzylamine in lieu of 4-methoxybenzylamine.

### Part D

The benzyl group was removed from the material from Part C using the method of Example 13 Part I. The crude product was purified by HPFC (silica gel eluted with a gradient of 10-30% methanol in dichloromethane) followed by recrystallization from ethyl acetate to provide 90 mg of 2-(2-methoxyethyl)-6,7-dimethyl-N¹-(tetrahydropyran-4-yl)-1H-imidazo[4,5-c]pyridine-1,4-diamine as white needles, mp 110-120° C. ¹H NMR (300 MHz, DMSO-d₆) δ 6.65 (d, J=1.4 Hz, 1H), 5.61 (br s, 2H), 3.90-3.73 (m, 4H), 3.30-2.84 (m, 8H), 2.43 (s, 3H), 2.27 (s, 3H), 1.61-1.22 (m, 4H); MS (ESI) m/z 320 (M+H)⁺; Anal. Calcd for C₁₆H₂₅N₅O₂.0.40H₂O: C, 58.84; H, 7.96; N, 21.44. Found: C, 58.92; H, 7.91; N, 21.51.

### Exemplary Compounds

Certain exemplary compounds, including some of those described above in the Examples, have the following Formulas (XIII, XIV, XV, or XVI) and the following R₁ and R₂ substituents, wherein each line of the table is matched with Formula XIII, XIV, XV, or XVI to represent a specific compound.

| | |
|---|---|
| | XIII |
| 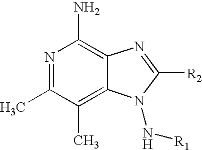 | |
| | XIV |
| 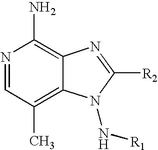 | |
| | XV |
| 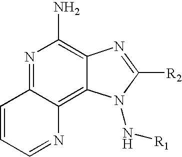 | |
| | XVI |
| 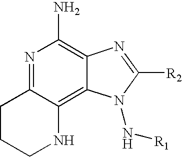 | |

| R₁ | R₂ |
|---|---|
| isopropyl | hydrogen |
| isopropyl | methyl |
| isopropyl | ethyl |
| isopropyl | n-propyl |
| isopropyl | n-butyl |
| isopropyl | methoxymethyl |
| isopropyl | ethoxymethyl |
| isopropyl | 2-methoxyethyl |
| isopropyl | hydroxymethyl |
| isopropyl | 2-hydroxyethyl |
| cyclohexyl | hydrogen |
| cyclohexyl | methyl |
| cyclohexyl | ethyl |
| cyclohexyl | n-propyl |
| cyclohexyl | n-butyl |
| cyclohexyl | methoxymethyl |
| cyclohexyl | ethoxymethyl |
| cyclohexyl | 2-methoxyethyl |
| cyclohexyl | hydroxymethyl |
| cyclohexyl | 2-hydroxyethyl |
| benzyl | hydrogen |
| benzyl | methyl |
| benzyl | ethyl |
| benzyl | n-propyl |
| benzyl | n-butyl |
| benzyl | methoxymethyl |
| benzyl | ethoxymethyl |
| benzyl | 2-methoxyethyl |
| benzyl | hydroxymethyl |
| benzyl | 2-hydroxyethyl |
| 3-phenylpropyl | hydrogen |
| 3-phenylpropyl | methyl |
| 3-phenylpropyl | ethyl |
| 3-phenylpropyl | n-propyl |
| 3-phenylpropyl | n-butyl |
| 3-phenylpropyl | methoxymethyl |
| 3-phenylpropyl | ethoxymethyl |
| 3-phenylpropyl | 2-methoxyethyl |
| 3-phenylpropyl | hydroxymethyl |
| 3-phenylpropyl | 2-hydroxyethyl |
| (pyridin-3-yl)methyl | hydrogen |
| (pyridin-3-yl)methyl | methyl |
| (pyridin-3-yl)methyl | ethyl |
| (pyridin-3-yl)methyl | n-propyl |
| (pyridin-3-yl)methyl | n-butyl |
| (pyridin-3-yl)methyl | methoxymethyl |
| (pyridin-3-yl)methyl | ethoxymethyl |
| (pyridin-3-yl)methyl | 2-methoxyethyl |
| (pyridin-3-yl)methyl | hydroxymethyl |
| (pyridin-3-yl)methyl | 2-hydroxyethyl |
| 3-[(methanesulfonyl)amino]propyl | hydrogen |
| 3-[(methanesulfonyl)amino]propyl | methyl |
| 3-[(methanesulfonyl)amino]propyl | ethyl |
| 3-[(methanesulfonyl)amino]propyl | n-propyl |
| 3-[(methanesulfonyl)amino]propyl | n-butyl |
| 3-[(methanesulfonyl)amino]propyl | methoxymethyl |
| 3-[(methanesulfonyl)amino]propyl | ethoxymethyl |
| 3-[(methanesulfonyl)amino]propyl | 2-methoxyethyl |
| 3-[(methanesulfonyl)amino]propyl | hydroxymethyl |
| 3-[(methanesulfonyl)amino]propyl | 2-hydroxyethyl |
| 3-(acetylamino)propyl | hydrogen |
| 3-(acetylamino)propyl | methyl |
| 3-(acetylamino)propyl | ethyl |
| 3-(acetylamino)propyl | n-propyl |
| 3-(acetylamino)propyl | n-butyl |
| 3-(acetylamino)propyl | methoxymethyl |
| 3-(acetylamino)propyl | ethoxymethyl |
| 3-(acetylamino)propyl | 2-methoxyethyl |
| 3-(acetylamino)propyl | hydroxymethyl |
| 3-(acetylamino)propyl | 2-hydroxyethyl |
| 3-[(isopropylcarbonyl)amino]propyl | hydrogen |
| 3-[(isopropylcarbonyl)amino]propyl | methyl |
| 3-[(isopropylcarbonyl)amino]propyl | ethyl |
| 3-[(isopropylcarbonyl)amino]propyl | n-propyl |
| 3-[(isopropylcarbonyl)amino]propyl | n-butyl |
| 3-[(isopropylcarbonyl)amino]propyl | methoxymethyl |
| 3-[(isopropylcarbonyl)amino]propyl | ethoxymethyl |
| 3-[(isopropylcarbonyl)amino]propyl | 2-methoxyethyl |
| 3-[(isopropylcarbonyl)amino]propyl | hydroxymethyl |
| 3-[(isopropylcarbonyl)amino]propyl | 2-hydroxyethyl |
| 3-[(cyclohexylcarbonyl)amino]propyl | hydrogen |
| 3-[(cyclohexylcarbonyl)amino]propyl | methyl |
| 3-[(cyclohexylcarbonyl)amino]propyl | ethyl |
| 3-[(cyclohexylcarbonyl)amino]propyl | n-propyl |
| 3-[(cyclohexylcarbonyl)amino]propyl | n-butyl |
| 3-[(cyclohexylcarbonyl)amino]propyl | methoxymethyl |
| 3-[(cyclohexylcarbonyl)amino]propyl | ethoxymethyl |
| 3-[(cyclohexylcarbonyl)amino]propyl | 2-methoxyethyl |
| 3-[(cyclohexylcarbonyl)amino]propyl | hydroxymethyl |
| 3-[(cyclohexylcarbonyl)amino]propyl | 2-hydroxyethyl |
| 3-[(morpholin-4-ylcarbonyl)amino]propyl | hydrogen |
| 3-[(morpholin-4-ylcarbonyl)amino]propyl | methyl |
| 3-[(morpholin-4-ylcarbonyl)amino]propyl | ethyl |
| 3-[(morpholin-4-ylcarbonyl)amino]propyl | n-propyl |
| 3-[(morpholin-4-ylcarbonyl)amino]propyl | n-butyl |
| 3-[(morpholin-4-ylcarbonyl)amino]propyl | methoxymethyl |
| 3-[(morpholin-4-ylcarbonyl)amino]propyl | ethoxymethyl |
| 3-[(morpholin-4-ylcarbonyl)amino]propyl | 2-methoxyethyl |
| 3-[(morpholin-4-ylcarbonyl)amino]propyl | hydroxymethyl |
| 3-[(morpholin-4-ylcarbonyl)amino]propyl | 2-hydroxyethyl |
| 3-{[(isopropylamino)carbonyl]amino}propyl | hydrogen |
| 3-{[(isopropylamino)carbonyl]amino}propyl | methyl |
| 3-{[(isopropylamino)carbonyl]amino}propyl | ethyl |
| 3-{[(isopropylamino)carbonyl]amino}propyl | n-propyl |
| 3-{[(isopropylamino)carbonyl]amino}propyl | n-butyl |
| 3-{[(isopropylamino)carbonyl]amino}propyl | methoxymethyl |
| 3-{[(isopropylamino)carbonyl]amino}propyl | ethoxymethyl |
| 3-{[(isopropylamino)carbonyl]amino}propyl | 2-methoxyethyl |
| 3-{[(isopropylamino)carbonyl]amino}propyl | hydroxymethyl |
| 3-{[(isopropylamino)carbonyl]amino}propyl | 2-hydroxyethyl |
| tetrahydropyran-4-yl | hydrogen |
| tetrahydropyran-4-yl | methyl |
| tetrahydropyran-4-yl | ethyl |
| tetrahydropyran-4-yl | n-propyl |
| tetrahydropyran-4-yl | n-butyl |
| tetrahydropyran-4-yl | methoxymethyl |
| tetrahydropyran-4-yl | ethoxymethyl |
| tetrahydropyran-4-yl | 2-methoxyethyl |
| tetrahydropyran-4-yl | hydroxymethyl |
| tetrahydropyran-4-yl | 2-hydroxyethyl |
| 3-(methylsulfonyl)propyl | hydrogen |
| 3-(methylsulfonyl)propyl | methyl |
| 3-(methylsulfonyl)propyl | ethyl |
| 3-(methylsulfonyl)propyl | n-propyl |
| 3-(methylsulfonyl)propyl | n-butyl |
| 3-(methylsulfonyl)propyl | methoxymethyl |
| 3-(methylsulfonyl)propyl | ethoxymethyl |
| 3-(methylsulfonyl)propyl | 2-methoxyethyl |
| 3-(methylsulfonyl)propyl | hydroxymethyl |
| 3-(methylsulfonyl)propyl | 2-hydroxyethyl |
| 2-(methylsulfonyl)ethyl | hydrogen |
| 2-(methylsulfonyl)ethyl | methyl |
| 2-(methylsulfonyl)ethyl | ethyl |
| 2-(methylsulfonyl)ethyl | n-propyl |
| 2-(methylsulfonyl)ethyl | n-butyl |
| 2-(methylsulfonyl)ethyl | methoxymethyl |
| 2-(methylsulfonyl)ethyl | ethoxymethyl |
| 2-(methylsulfonyl)ethyl | 2-methoxyethyl |
| 2-(methylsulfonyl)ethyl | hydroxymethyl |
| 2-(methylsulfonyl)ethyl | 2-hydroxyethyl |
| 1-(methylsulfonyl)piperidin-4-yl | hydrogen |
| 1-(methylsulfonyl)piperidin-4-yl | methyl |
| 1-(methylsulfonyl)piperidin-4-yl | ethyl |
| 1-(methylsulfonyl)piperidin-4-yl | n-propyl |
| 1-(methylsulfonyl)piperidin-4-yl | n-butyl |
| 1-(methylsulfonyl)piperidin-4-yl | methoxymethyl |
| 1-(methylsulfonyl)piperidin-4-yl | ethoxymethyl |
| 1-(methylsulfonyl)piperidin-4-yl | 2-methoxyethyl |
| 1-(methylsulfonyl)piperidin-4-yl | hydroxymethyl |
| 1-(methylsulfonyl)piperidin-4-yl | 2-hydroxyethyl |
| 1-acetylpiperidin-4-yl | hydrogen |
| 1-acetylpiperidin-4-yl | methyl |
| 1-acetylpiperidin-4-yl | ethyl |
| 1-acetylpiperidin-4-yl | n-propyl |
| 1-acetylpiperidin-4-yl | n-butyl |
| 1-acetylpiperidin-4-yl | methoxymethyl |
| 1-acetylpiperidin-4-yl | ethoxymethyl |
| 1-acetylpiperidin-4-yl | 2-methoxyethyl |
| 1-acetylpiperidin-4-yl | hydroxymethyl |
| 1-acetylpiperidin-4-yl | 2-hydroxyethyl |
| 1-(isopropylcarbonyl)piperidin-4-yl | hydrogen |
| 1-(isopropylcarbonyl)piperidin-4-yl | methyl |
| 1-(isopropylcarbonyl)piperidin-4-yl | ethyl |
| 1-(isopropylcarbonyl)piperidin-4-yl | n-propyl |
| 1-(isopropylcarbonyl)piperidin-4-yl | n-butyl |
| 1-(isopropylcarbonyl)piperidin-4-yl | methoxymethyl |
| 1-(isopropylcarbonyl)piperidin-4-yl | ethoxymethyl |
| 1-(isopropylcarbonyl)piperidin-4-yl | 2-methoxyethyl |
| 1-(isopropylcarbonyl)piperidin-4-yl | hydroxymethyl |
| 1-(isopropylcarbonyl)piperidin-4-yl | 2-hydroxyethyl |
| 1-(morpholin-4-ylcarbonyl)piperidin-4-yl | hydrogen |
| 1-(morpholin-4-ylcarbonyl)piperidin-4-yl | methyl |
| 1-(morpholin-4-ylcarbonyl)piperidin-4-yl | ethyl |
| 1-(morpholin-4-ylcarbonyl)piperidin-4-yl | n-propyl |
| 1-(morpholin-4-ylcarbonyl)piperidin-4-yl | n-butyl |
| 1-(morpholin-4-ylcarbonyl)piperidin-4-yl | methoxymethyl |
| 1-(morpholin-4-ylcarbonyl)piperidin-4-yl | ethoxymethyl |
| 1-(morpholin-4-ylcarbonyl)piperidin-4-yl | 2-methoxyethyl |
| 1-(morpholin-4-ylcarbonyl)piperidin-4-yl | hydroxymethyl |
| 1-(morpholin-4-ylcarbonyl)piperidin-4-yl | 2-hydroxyethyl |
| 1-[(isopropylamino)carbonyl]piperidin-4-yl | hydrogen |
| 1-[(isopropylamino)carbonyl]piperidin-4-yl | methyl |
| 1-[(isopropylamino)carbonyl]piperidin-4-yl | ethyl |
| 1-[(isopropylamino)carbonyl]piperidin-4-yl | n-propyl |
| 1-[(isopropylamino)carbonyl]piperidin-4-yl | n-butyl |
| 1-[(isopropylamino)carbonyl]piperidin-4-yl | methoxymethyl |
| 1-[(isopropylamino)carbonyl]piperidin-4-yl | ethoxymethyl |
| 1-[(isopropylamino)carbonyl]piperidin-4-yl | 2-methoxyethyl |
| 1-[(isopropylamino)carbonyl]piperidin-4-yl | hydroxymethyl |
| 1-[(isopropylamino)carbonyl]piperidin-4-yl | 2-hydroxyethyl |
| cyclobutyl | hydrogen |
| cyclobutyl | methyl |
| cyclobutyl | ethyl |
| cyclobutyl | n-propyl |
| cyclobutyl | n-butyl |
| cyclobutyl | methoxymethyl |
| cyclobutyl | ethoxymethyl |
| cyclobutyl | 2-methoxyethyl |
| cyclobutyl | hydroxymethyl |
| cyclobutyl | 2-hydroxyethyl |
| cyclopentyl | hydrogen |
| cyclopentyl | methyl |
| cyclopentyl | ethyl |
| cyclopentyl | n-propyl |
| cyclopentyl | n-butyl |
| cyclopentyl | methoxymethyl |
| cyclopentyl | ethoxymethyl |
| cyclopentyl | 2-methoxyethyl |
| cyclopentyl | hydroxymethyl |
| cyclopentyl | 2-hydroxyethyl |
| 2-[(methanesulfonyl)amino]ethyl | hydrogen |
| 2-[(methanesulfonyl)amino]ethyl | methyl |
| 2-[(methanesulfonyl)amino]ethyl | ethyl |
| 2-[(methanesulfonyl)amino]ethyl | n-propyl |
| 2-[(methanesulfonyl)amino]ethyl | n-butyl |
| 2-[(methanesulfonyl)amino]ethyl | methoxymethyl |
| 2-[(methanesulfonyl)amino]ethyl | ethoxymethyl |
| 2-[(methanesulfonyl)amino]ethyl | 2-methoxyethyl |
| 2-[(methanesulfonyl)amino]ethyl | hydroxymethyl |
| 2-[(methanesulfonyl)amino]ethyl | 2-hydroxyethyl |

Compounds of the invention have been found to modulate cytokine biosynthesis by inducing the production of interferon α and/or tumor necrosis factor α in human cells when tested using the method described below.

### Cytokine Induction in Human Cells

An in vitro human blood cell system is used to assess cytokine induction. Activity is based on the measurement of interferon (α) and tumor necrosis factor (α) (IFN-α and TNF-α, respectively) secreted into culture media as described by Testerman et al. in “Cytokine Induction by the Immunomodulators Imiquimod and S-27609,” Journal of Leukocyte Biology, 58, 365-372 (September, 1995).

### Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

### Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

### Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 hours to 24 hours at 37° C. in a 5% carbon dioxide atmosphere.

### Separation

Following incubation the plates are centrifuged for 10 minutes at 1000 rpm (approximately 200×g) at 4° C. The cell-free culture supernatant is removed and transferred to sterile polypropylene tubes. Samples are maintained at −30° C. to −70° C. until analysis. The samples are analyzed for IFN-α by ELISA and for TNF-α by IGEN/BioVeris Assay.

### Interferon (α) and Tumor Necrosis Factor (α) Analysis

IFN-α concentration is determined with a human multi-subtype colorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

### Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as “*”, and is noted as not reliably detectable. In subsequent calculations and statistics, “*”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (1 molar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### Cytokine Induction in Human Cells

### High Throughput Screen

The CYTOKINE INDUCTION IN HUMAN CELLS test method described above was modified as follows for high throughout screening.

### Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete (2-fold the final cell density). The PBMC suspension is added to 96-well flat bottom sterile tissue culture plates.

### Compound Preparation

The compounds are solubilized in dimethyl sulfoxide (DMSO). The compounds are generally tested at concentrations ranging from 30-0.014 μM. Controls include cell samples with media only, cell samples with DMSO only (no compound), and cell samples with a reference compound 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) on each plate. The solution of test compound is added at 7.5 mM to the first well of a dosing plate and serial 3 fold dilutions are made for the 7 subsequent concentrations in DMSO. RPMI Complete media is then added to the test compound dilutions in order to reach a final compound concentration of 2-fold higher (60-0.028 μM) than the final tested concentration range.

### Incubation

Compound solution is then added to the wells containing the PBMC suspension bringing the test compound concentrations to the desired range (usually 30-0.014 μM) and the DMSO concentration to 0.4%. The final concentration of PBMC suspension is 2×10⁶ cells/mL. The plates are covered with sterile plastic lids, mixed gently and then incubated for 18 to 24 hours at 37° C. in a 5% carbon dioxide atmosphere.

### Separation

Following incubation the plates are centrifuged for 10 minutes at 1000 rpm (approximately 200 g) at 4° C. 4-plex Human Panel MSD Multi-Spot® 96-well plates are pre-coated with the appropriate capture antibodies by MesoScale Discovery, Inc. (MSD, Gaithersburg, Md.). The cell-free culture supernatants are removed and transferred to the MSD plates. Fresh samples are typically tested, although they may be maintained at −30 to −70° C. until analysis.

### Interferon-α and Tumor Necrosis Factor-α Analysis

MSD Multi-Spot® plates contain within each well capture antibodies for human TNF-α and human IFN-α that have been pre-coated on specific spots. Each well contains four spots: one human TNF-α capture antibody (MSD) spot, one human IFN-α capture antibody (PBL Biomedical Laboratories, Piscataway, N.J.) spot, and two inactive bovine serum albumin spots. The human TNF-α capture and detection antibody pair is from MesoScale Discovery. The human IFN-α multi-subtype antibody (PBL Biomedical Laboratories) captures all IFN-α subtypes except IFN-α F (IFNA21). Standards consist of recombinant human TNF-α (R&D Systems, Minneapolis, Minn.) and IFN-α (PBL Biomedical Laboratories). Samples and separate standards are added at the time of analysis to each MSD plate. Two human IFN-α detection antibodies (Cat. Nos. 21112 & 21100, PBL) are used in a two to one ratio (weight:weight) to each other to determine the IFN-α concentrations. The cytokine-specific detection antibodies are labeled with the Sulfo-TAG™ reagent (MSD). After adding the Sulfo-TAG™ labeled detection antibodies to the wells, each well's electrochemoluminescent levels are read using MSD's Sector HTS Reader™. Results are expressed in pg/mL upon calculation with known cytokine standards.

### Assay Data and Analysis

In total, the data output of the assay consists of concentration values of TNF-α or IFN-α (y-axis) as a function of compound concentration (x-axis).

A plate-wise scaling is performed within a given experiment aimed at reducing plate-to-plate variability associated within the same experiment. First, the greater of the median DMSO (DMSO control wells) or the experimental background (usually 20 pg/mL for IFN-α and 40 pg/mL for TNF-α) is subtracted from each reading. Negative values that may result from background subtraction are set to zero. Each plate within a given experiment has a reference compound that serves as a control. This control is used to calculate a median expected area under the curve across all plates in the assay. A plate-wise scaling factor is calculated for each plate as a ratio of the area of the reference compound on the particular plate to the median expected area for the entire experiment. The data from each plate are then multiplied by the plate-wise scaling factor for all plates. Only data from plates bearing a scaling factor of between 0.5 and 2.0 (for both cytokines IFN-α, TNF-α) are reported. Data from plates with scaling factors outside the above mentioned interval are retested until they bear scaling factors inside the above mentioned interval. The above method produces a scaling of the y-values without altering the shape of the curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91). The median expected area is the median area across all plates that are part of a given experiment.

A second scaling may also be performed to reduce inter-experiment variability (across multiple experiments). All background-subtracted values are multiplied by a single adjustment ratio to decrease experiment-to-experiment variability. The adjustment ratio is the area of the reference compound in the new experiment divided by the expected area of the reference compound based on an average of previous experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from an average of previous experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. (canceled)

2. A compound of the following Formula II: 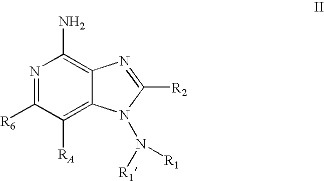
wherein:
R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;
R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and 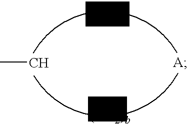
or R₁′ and R₁ together with the nitrogen atom to which they are bonded can join to form a group selected from the group consisting of: 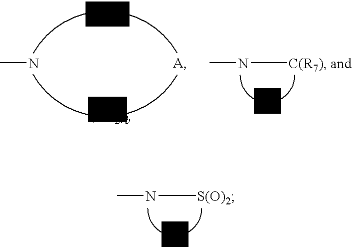
R₂ is selected from the group consisting of:
hydrogen,
alkyl,
alkenyl,
aryl,
heteroaryl,
heterocyclyl,
alk-yl-Z-alkylenyl,
aryl-Z-alkylenyl,
alkenyl-Z-alkylenyl, and
alkyl or alkenyl substituted by one or more substituents selected from the group consisting of:
hydroxy,
halogen,
—N(R₆)₂,
—C(R₇)—N(R₆)₂,
—S(O)₂—N(R₆)₂,
—N(R₆)—C(R₇)—C₁₋₁₀ alkyl,
—N(R₆)—C(R₇)-aryl,
—N(R₆)—S(O)₂—C₁₋₁₀-alkyl,
—N(R₆)—S(O)₂-aryl,
—C(O)—C₁₋₁₀ alkyl,
—C(O)—O—C₁₋₁₀ alkyl,
—O—C(R₇)—C₁₋₁₀ alkyl,
—O—C(R₇)-aryl,
—O—C(R₇)—N(R₆)—C₁₋₁₀ alkyl,
—O—C(R₇)—N(R₆)-aryl,
—N₃,
aryl,
heteroaryl,
heterocyclyl,
—C(O)-aryl, and
—C(O)-heteroaryl;
R_{A} and R_{B} are each independently selected from the group consisting of:
hydrogen,
halogen,
alkyl,
alkenyl,
alkoxy,
alkylthio, and
—N(R₁₂)₂;
or when taken together, R_{A} and R_{B} form a fused pyridine ring which is unsubstituted or substituted by one or more R groups, or substituted by one R₃ group, or substituted by one R₃ group and one R group, or substituted by one R₃ group and two R groups;
or when taken together, R_{A} and R_{B} form a fused tetrahydropyridine
ring which is unsubstituted or substituted by one or more R groups;
R is selected from the group consisting of:
halogen,
hydroxy,
alkyl,
alkenyl,
haloalkyl,
alkoxy,
alkylthio, and
—N(R₁₂)₂;
R₃ is selected from the group consisting of:
-Z′-R₄′,
-Z′-X′-R₄′,
-Z′-X′—Y′—R₄′, and
-Z′-X′-R₅′;
R₄ is selected from the group consisting of hydrogen, alkyl, alkenyl, alkynyl, aryl, arylalkylenyl, heteroaryl, and heterocyclyl wherein the alk-yl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;
R₅ is selected from the group consisting of: 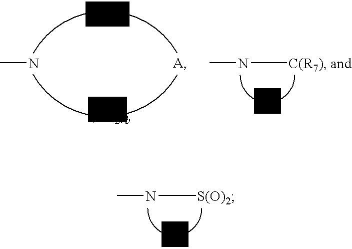
X is C₂₋₂₀ alkylene;
Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group 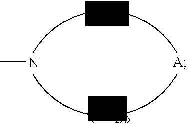
Z is selected from the group consisting of —O— and —S(O)₀₋₂—;
A is selected from the group consisting of —CH(R₆)—, —O—, —N(R₆)-, —N(Y—R₄)-, and —N(X—N(R₆)—Y—R₄)-,
a and b are independently integers from 1 to 4 with the proviso that when
A is —O—, —N(R₆)-, —N(Y—R₄)-, or —N(X—N(R₆)—Y—R₄)- then a and b are independently integers from 2 to 4;
R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅′ is selected from the group consisting of: 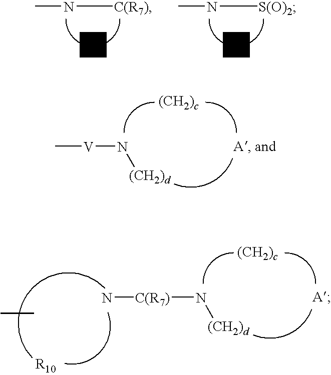
X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups;
Y′ is selected from the group consisting of:
—S(O)₀₋₂—,
—S(O)₂—N(R₁₁)-,
—C(R₇)—,
—C(R₇)—O—,
—O—C(R₇)-,
—O—C(O)—O—,
—N(R₁₁)-Q-,
—C(R₇)—N(R₁₁)-,
—O—C(R₇)—N(R₁₁)-,
—C(R₇)—N(OR₁₂)-, 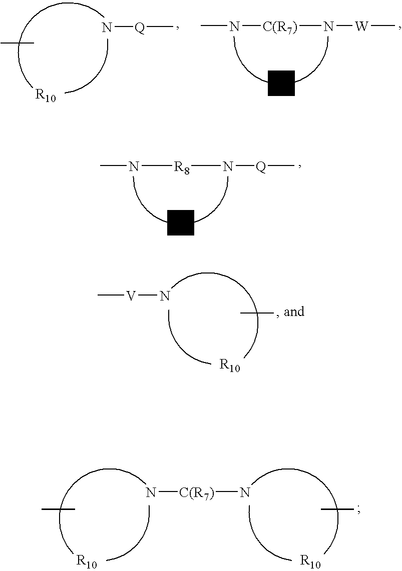
Z′ is a bond or —O—;
A′ is selected from the group consisting of —CH₂—, —O—, —C(O)—, —S(O)₀₋₂—, and —N(R₄′)-;
Q is selected from the group consisting of a bond, —C(R₇)-, —C(R₇)—C(R₇)-, —S(O)₂—, —C(R₇)—N(R₁₁)—W—, —S(O)₂—N(R₁₁)-, —C(R₇)—O—, and —C(R₇)—N(OR₁₂)-,
V is selected from the group consisting of —C(R₇)-, —O—C(R₇)-, —N(R₁₁)—C(R₇)-, and —S(O)₂—;
W is selected from the group consisting of a bond, —C(O)—, and —S(O)₂—;
c and d are independently integers from 1 to 6 with the proviso that c+d is ≦7, and when A′ is —O— or —N(R₄′)- then c and d are independently integers from 2 to 4;
R₆ is selected from the group consisting of hydrogen, alkyl, and arylalkylenyl;
R₇ is selected from the group consisting of ═O and ═S;
R₈ is C₂₋₇ alkylene;
R₁₀ is C₃₋₈ alkylene;
R₁₁ is selected from the group consisting of hydrogen, C₁₋₁₀ alkyl, C₂₋₁₀ alkenyl, C₁₋₁₀ alkoxyC₂₋₁₀ alkylenyl, and arylC₁₋₁₀ alkylenyl; and
R₁₂ is selected from the group consisting of hydrogen and alkyl;
or a pharmaceutically acceptable salt thereof.

3. A compound of the Formula III: 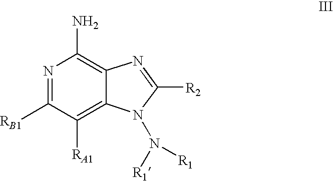
wherein:
R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;
R₁ is selected from the group consisting of:
-R₄,
—Y—R₄,
-X—R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄,
-X—S(O)₂—R₄, and 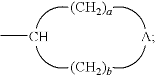
or R₁′ and R₁ together with the nitrogen atom to which they are bonded can join to form a group selected from the group consisting of: 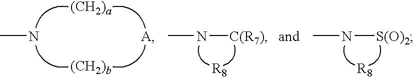
R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
hydroxy,
halogen,
—N(R₆)₂,
—C(R₇)—N(R₆)₂,
—S(O)₂—N(R₆)₂,
—N(R₆)—C(R₇)—C₁₋₁₀ alkyl,
—N(R₆)—C(R₇)-aryl,
—N(R₆)—S(O)₂—C₁₋₁₀ alkyl,
—N(R₆)—S(O)₂-aryl,
—C(O)—C₁₋₁₀ alkyl,
—C(O)—O—C₁₋₁₀ alkyl,
—O—C(R₇)—C₁₋₁₀ alkyl,
—O—C(R₇)-aryl,
—O—C(R₇)—N(R₆)—C₁₋₁₀ alkyl,
—O—C(R₇)—N(R₆)-aryl,
—N₃,
aryl,
heteroaryl,
heterocyclyl,
—C(O)-aryl, and
—C(O)-heteroaryl;
R_{A1} and R_{B1} are each independently selected from the group consisting of:
hydrogen,
halogen,
alkyl,
alkenyl,
alkoxy,
alkylthio, and
—N(R₁₂)₂;
R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;
R₅ is selected from the group consisting of; 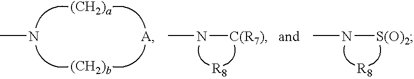
R₆ is selected from the group consisting of hydrogen, alkyl, and arylalkylenyl;
R₇ is selected from the group consisting of ═O and ═S;
R₈ is C₂₋₇ alkylene;
R₁₂ is selected from the group consisting of hydrogen and alkyl;
A is selected from the group consisting of —CH(R₆)-, —O—, —N(R₆)-, —N(Y—R₄)-, and —N(X—N(R₆)—Y—R₄)-;
X is C₂₋₂₀ alkylene;
Y is selected from the group consisting of —C(R₇)-, —C(R₇)—O—, —S(O)₂—, —S(O)_{z}—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group 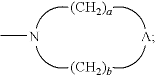
Z is selected from the group consisting of —O— and —S(O)₀₋₂—; and
a and b are independently integers from 1 to 4 with the proviso that when
A is —O—, —N(R₆)-, —N(Y—R₄)-, or —N(X—N(R₆)—Y—R₄)- then a and b are independently integers from 2 to 4;
or a pharmaceutically acceptable salt thereof.

4. A compound of the Formula IV: 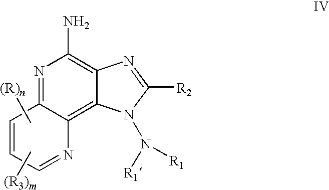
wherein:
R₁′ is selected from the group consisting of hydrogen, alkyl, hydroxyalkyl, and alkoxyalkyl wherein the alkyl group contains at least 2 carbon atoms between the hydroxy or alkoxy substituent and the nitrogen atom to which R₁′ is bonded;
R₁ is selected from the group consisting of:
-R₄,
—Y—R₄,
-X-R₅,
-X—N(R₆)—Y—R₄,
-X—C(R₇)—N(R₆)-R₄,
-X—O—C(R₇)—N(R₆)-R₄,
-X—S(O)₂—N(R₆)-R₄,
-X—O—R₄
-X—S(O)₂—R₄, and 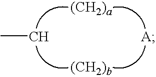
or R₁′ and R₁ together with the nitrogen atom to which they are bonded can join to form a group selected from the group consisting of: 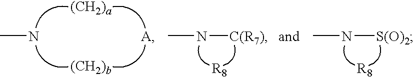
R₂ is selected from the group consisting of:
hydrogen,
alkyl,
alkenyl,
aryl,
heteroaryl,
heterocyclyl,
alkyl-Z-alkylenyl,
aryl-Z-alkylenyl,
alkenyl-Z-alkylenyl, and
alkyl or alkenyl substituted by one or more substituents selected from the group consisting of:
hydroxy,
halogen,
—N(R₆)₂,
—C(R₇)—N(R₆)₂,
—S(O)₂—N(R₆)₂,
—N(R₆)—C(R₇)—C₁₋₁₀ alkyl,
—N(R₆)—C(R₇)-aryl,
—N(R₆)—S(O)₂—C₁₋₁₀ alkyl,
—N(R₆)—S(O)₂-aryl,
—C(O)—C₁₋₁₀ alkyl,
—C(O)—O—C₁₋₁₀ alkyl,
—O—C(R₇)—C₁₋₁₀ alkyl,
—O—C(R₇)-aryl,
—O—C(R₇)—N(R₆)—C₁₋₁₀ alkyl,
—O—C(R₇)—N(R₆)-aryl,
—N₃,
aryl,
hetetoaryl,
heterocyclyl,
—C(O)-aryl, and
—C(O)-heteroaryl;
R is selected from the group consisting of:
halogen,
hydroxy,
alkyl,
alkenyl,
haloalkyl,
alkoxy,
alkylthio, and
—N(R₁₂)₂;
R₃ is selected from the group consisting of:
-Z′-R₄′,
-Z′-X′-R₄′,
-Z′-X′—Y′—R′, and
-Z′-X′-R₅′;
n is an integer from 0 to 3;
m is 0 or 1, with the proviso that when m is 1, n is 0, 1, or 2;
R₄ is selected from the group consisting of hydrogen, alkyl, alkenyl, alkynyl, aryl, arylalkylenyl, heteroaryl, and heterocyclyl wherein the alkyl, alkenyl, alkynyl, aryl, arylalkylenyl, heteroaryl, and heterocyclyl groups can be unsubstituted or substituted by one or more substituents independently selected from the group consisting of alkyl, alkoxy, haloalkyl, haloalkoxy, halogen, nitro, hydroxy, mercapto, cyano, carboxy, formyl, aryl, aryloxy, arylalkoxy, heteroaryl, heteroaryloxy, heteroarylalkoxy, heterocyclyl, heterocyclylalkylenyl, amino, alkylamino, (arylalkylenyl)amino, dialkylamino, and in the case of alkyl, alkenyl, alkynyl, and heterocyclyl, oxo, with the proviso that when R₄ is a substituted alkyl group and the substituent contains a hetero atom which bonds directly to the alkyl group then the alkyl group contains at least two carbons between the substituent and the nitrogen atom to which R₁ is bonded;
R₅ is selected from the group consisting of: 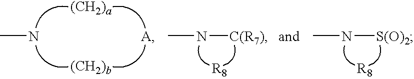
X is C₂₋₂₀ alkylene;
Y is selected from the group consisting of —C(R₇)-, —C(R₇)—O—, —S(O)₂—, —S(O)₂—N(R₆)-, and —C(R₇)—N(R₉)-; wherein R₉ is selected from the group consisting of hydrogen, alkyl, and arylalkylenyl; or R₉ and R₄ together with the nitrogen atom to which R₉ is bonded can join to form the group 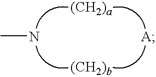
Z is selected from the group consisting of —O— and —S(O)₀₋₂—;
A is selected from the group consisting of —CH(R₆)-, —O—, —N(R₆)-, —N(Y—R₄)-, and —N(X—N(R₆)—Y—R₄)-;
a and b are independently integers from 1 to 4 with the proviso that when
A is —O—, —N(R₆)-, —N(Y—R₄)-, or —N(X—N(R₆)—Y—R₄)- then a and b are independently integers from 2 to 4;
R₄′ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅′ is selected from the group consisting of: 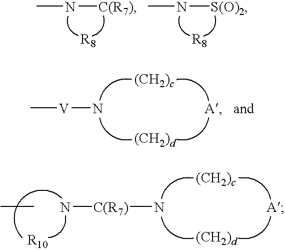
X′ is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene, or heterocyclylene and optionally interrupted by one or more —O— groups;
Y′ is selected from the group consisting of:
—S(O)₀₋₂—,
—S(O)₂—N(R₁₁)-,
—C(R₇)-,
—(R₇)—O—,
—O—C(R₇)-,
—O—C(O)—O—,
—N(R₁₁)—O—,
—C(R₇)—N(R₁₁)-,
—O—C(R₇)—N(R₁₁)-,
—C(R₇)—N(OR₁₂)-, 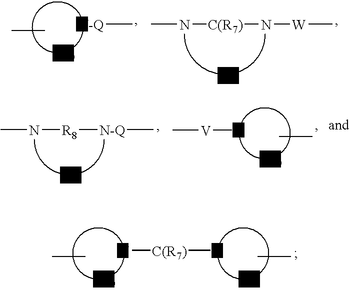
Z′ is a bond or —O—;
A′ is selected from the group consisting of —CH₂—, —O—, —C(O)—, —S(O)₀₋₂—, and —N(R₄′)-;
Q is selected from the group consisting of a bond, —C(R₇)-, —C(R₇)—C(R₇)-, —S(O)₂—, —C(R₇)—N(R₁₁)—W—, —S(O)₂—N(R₁₁)-, —C(R₇)—O—, and —C(R₇)—N(OR₁₂)-;
V is selected from the group consisting of —C(R₇)—, —O—C(R₇)-, —N(R₁₁)—C(R₇)-, and —S(O)₂—;
W is selected from the group consisting of a bond, —C(O)—, and —S(O)₂—;
c and d are independently integers from 1 to 6 with the proviso that c+d is ≦7, and when A′ is —O— or —N(R₄)- then c and d are independently integers from 2 to 4;
R₆ is selected from the group consisting of hydrogen, alkyl, and arylalkylenyl;
R₇ is selected from the group consisting of ═O and ═S;
R₈ is C₂₋₇ allylene;
R₁₀ is C₃₋₈ alkylene;
R₁₁ is selected from the group consisting of hydrogen, C₁₋₁₀alkyl, C₂₋₁₀alkenyl, C₁₋₁₀ alkoxyC₂₋₁₀ alkylenyl, and arylC₁₋₁₀ alkylenyl; and
R₁₂ is selected from the group consisting of hydrogen and alkyl;
or a pharmaceutically acceptable salt thereof.

5-6. (canceled)

7. The compound or salt according to claim 3 wherein R_{A1} and R_{B1} are each independently selected from hydrogen and alkyl.

8. The compound or salt according to claim 7 wherein R_{A1} and R_{B1} are each methyl.

9. (canceled)

10. The compound or salt according to claim 4 wherein m is 0, n is 0.

11-17. (canceled)

18. The compound or salt according to claim 3 wherein R₂ is selected from the group consisting of hydrogen, alkyl, hydroxyalkyl, and alkoxyalkylenyl.

19. (canceled)

20. The compound or salt according to claim 3 wherein R₁′ is hydrogen or alkyl.

21-24. (canceled)

25. The compound or salt according to claim 3 wherein R₁ is -R₄, and -R₄ is C₂₋₆ alkyl.

26-29. (canceled)

30. The compound or salt according to claim 3 wherein R₁ is
-X—N(R₆)—Y—R₄ wherein:
X is C₂₋₄ alkylene;
R₆ is hydrogen or C₁₋₄ alkyl;
Y is selected from the group consisting of —C(O)—, —S(O)₂—, and —C(O)—NH—; and
R₄ is C₁₋₆ alkyl, phenyl, or pyridyl
wherein the phenyl or pyridyl groups are optionally substituted with one or more substituents independently selected from the group consisting of alkyl, alkoxy, hydroxy, halogen, cyano, and alkylamino; or —Y—R₄ is

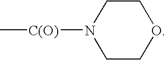

31. The compound or salt according to claim 3 wherein R₁ is

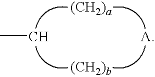

32. A pharmaceutical composition comprising a therapeutically effective amount of a compound or salt of claim 3 in combination with a pharmaceutically acceptable carrier.

33. A method of inducing cytokine biosynthesis in an animal comprising administering an effective amount of a compound or salt according to claim 3 the animal.

34-35. (canceled)

36. The compound or salt according to claim 4 wherein R₂ is selected from the group consisting of hydrogen, alkyl, hydroxyalkyl, and alkoxyalkylenyl.

37. The compound or salt according to claim 4 wherein R₁′ is hydrogen or alkyl.

38. The compound or salt according to claim 4 wherein R₁ is -R₄, and -R₄ is C₂₋₆ alkyl.

39. The compound or salt according to claim 4 wherein R₁ is -X—N(R₆)—Y—R₄ wherein:
X is C₂₋₄ alkylene;
R₆ is hydrogen or C₁₋₄ alkyl;
Y is selected from the group consisting of —C(O)—, —S(O)₂—, and —C(O)—NH—; and
R₄ is C₁₋₆ alkyl, phenyl, or pyridyl;
wherein the phenyl or pyridyl groups are optionally substituted with one or more substituents independently selected from the group consisting of alkyl, alkoxy, hydroxy, halogen, cyano, and alkylamino; or —Y—R₄ is

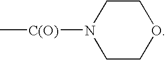

40. The compound or salt according to claim 4 wherein R₁ is

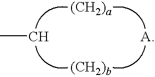

41. A pharmaceutical composition comprising a therapeutically effective amount of a compound or salt of claim 4 in combination with a pharmaceutically acceptable carrier.

42. A method of inducing cytokine biosynthesis in an animal comprising administering an effective amount of a compound or salt according to claim 4 to the animal.
